(19) United States
(12) Patent Application Publication
(10) Pub. No.: US 2026/0072036 A1
(43) Pub. Date: Mar. 12, 2026

(54) BICYCLONONYNE REAGENTS FOR CELL IMAGING

(71) Applicant: The General Hospital Corporation, Boston, MA (US)

(72) Inventors: Ralph Weissleder, Peabody, MA (US); Jonathan C. Carlson, Boston, MA (US)

(21) Appl. No.: 18/871,954

(22) PCT Filed: Jun. 12, 2023
(86) PCT No.: PCT/US2023/068279

§ 371 Date: Dec. 5, 2024

Related U.S. Application Data
(60) Provisional application No. 63/353,020, filed on Jun. 16, 2022.

(51) Int. Cl. G01N 33/58; C07C 271/16; C07D 311/82; C07D 405/12; C07K 1/107; G01N 21/64

(52) U.S. Cl. CPC G01N 33/582; C07C 271/16; C07D 311/82; C07D 405/12; C07K 1/1077; G01N 21/6428; G01N 2021/6432

(57) ABSTRACT

The present disclosure provides bicyclononyne based compounds and methods to prepare an antibody conjugate with a fluorophore, as well as the methods of using these conjugates for cellular imaging. In one example, the conjugate may be coupled with a quencher to absorb fluorescence from the fluorophore.

### CLAIM OF PRIORITY

This application claims priority to U.S. Provisional Patent Application Ser. No. 63/353,020, filed on Jun. 16, 2022, the entire contents of which are hereby incorporated by reference.

### TECHNICAL FIELD

This invention relates to tridentate ligands containing a bicyclononyne (BCN) moiety, and methods of using these ligands, e.g., for cellular fluorescence imaging, including multiplexed cellular fluorescence imaging.

### BACKGROUND

Processing cellular samples, e.g., for immunostaining and image cytometry, often can be quite challenging. One drawback is that the cellular samples are often scant (often <1,000 cells per pass from a fine needle aspirate), limiting the number of special stains that can be done, and also delicate, lacking the structural scaffold of intact tissue architecture. Even when processed with fluorescent antibodies, the number of different stains for cellular samples is typically limited to 4-6 and is often insufficient for in depth cancer cell profiling for diagnosis or treatment assessment. This limitation also extends to immune profiling, where significantly more than 4-6 markers need to be interrogated so that analysis reflects the representative immunocyte populations in the tumor microenvironment.

### SUMMARY

Most fluorescent cycling methods were originally developed for paraffin embedded tissue sections that can withstand harsh destaining/quenching conditions. Unfortunately, these harsh conditions often requiring oxidants for bleaching are not compatible with cellular samples such as those in fine needle aspirates (FNA). Furthermore, it is not uncommon for antibody-DNA cycling technologies to take hours-days of sample processing. Disclosed herein, inter alia, is an ultrafast method of single cell cycling by using fluorophore-based linkers containing a bicyclononyne (BCN)-based clickable moiety in their structure:

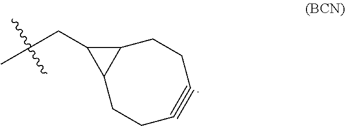 [SMILES: *CC1C2CCC#CCCC12]

In one example, a tetrazine- or an azide-functionalized quencher can be clicked to the BCN moiety, thereby quenching fluorescence and turning off the fluorophore.

Advantageously and unexpectedly, the BCN-based probes within the present claims show superior oxidation- and photo-stability after environmental exposure (e.g., ambient light, microscopy illumination, and air) and higher quenching performance compared to trans-cyclooctene-based probes, such as rTCO, TCO, cTCO, and dTCO. The reasons for these superior results are thus-far unknown, hence, the results for the BCN-based probes described in this application could not be predicted on the basis of the TCO-based probes ahead of testing and experimentation. Because all exemplified probes result in ligation of a quencher adjacent to fluorophore with identical and flexible PEG4 linker, a similar quenching performance could be expected. Nevertheless, and in sharp contrast, as the experimental data shows, the observed quenching varies strikingly for the BCN probes within the present claims showing far superior performance. The performance of BCN probes is unexpectedly similar to or better than the cyclopropene (CP)-based probes, which are generally more stable due to known reduced reactivity of three-membered rings (including reactions with light and oxygen) vs the eight-membered rings. As the experimental data shows, the BCN-based probes within the present claims exhibit the lowest residual fluorescence after exposure to ambient light and oxygen followed by quenching. In other words, the BCN probes are significantly more stable compared to TCO counterparts and comparable even to CP-counterparts under routine imaging conditions (e.g., microscope illumination) and retain their reactivity and performance characteristics in the quenching click reaction after exposure to common storage and handling protocols as well as the benchtop atmosphere. The BCN probes also avoid the possibility of chemical degradation and release of either the fluorophore or the quencher that is common for rTCO-based probes, which (despite its potential pathway for quencher disconnection/release) shows best quenching performance among the TCO-type probes.

Furthermore, in the tetrazine/cyclooctyne reaction, the BCN-labeled antibody probes within the instant claims show significantly enhanced (about 1400×) acceleration of the quenching reaction as compared to the predicted kinetics for the Tz/BCN reaction alone, which translates the expected time for complete reaction at the experimental concentration from months to hours. In fact, as the experimental data in the present application shows, at a concentration of a tetrazine-based quencher as low as 1 μM, complete quenching of cells stained with BCN- and fluorophore-labeled antibodies was observed within just two minutes. That is, an octyne/tetrazine click reaction that would be expected to take dozens of hours can be accelerated to 2-3 minutes using the probes within the present claims in the biological context. In a reaction with azide-containing quenchers, in the cellular context, the BCN-based antibody probes showed even more dramatic (in fact >5,000-fold) reaction acceleration vs predicted rate for the underlying azide-alkyne reaction kinetics. This further allows to translate the reaction timeframe to just a few minutes at routine imaging conditions. This dramatic enhancement of the reaction rate could not be predicted ahead of experimentation.

Due to their enhanced stability and superior reaction kinetics in both the tetrazine- and the azide-click reactions, the BCN-based probes within the present claims allow ultra-fast (<1 sec) quenching of fluorescence in clinical specimens with multichannel imaging of 20-30 markers within just one hour.

In one general aspect, the present disclosure provides a compound of Formula (I):

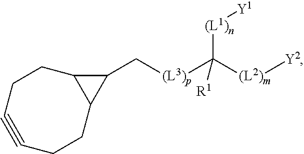

or a pharmaceutically acceptable salt thereof, wherein:
R¹ is selected from H, C₁₋₃ alkyl, and C₁₋₃ haloalkyl;
each L¹ is independently selected from N(R^{N}), O, C(═O), S(═O)₂, C₁₋₆ alkylene, —(OCH₂CH₂)ₓ—, and —(CH₂CH₂O)ₓ—;
n is an integer from 1 to 10;
each L² is independently selected from N(R^{N}), O, C(═O), S(═O)₂, C₁₋₆ alkylene, —(OCH₂CH₂)ₓ—, and —(CH₂CH₂O)ₓ—;
m is an integer from 1 to 10;
each L³ is independently selected from N(R^{N}), O, C(═O), S(═O)₂, C₁₋₆ alkylene, —(OCH₂CH₂)ₓ—, and —(CH₂CH₂O)ₓ—;
wherein each of said C₁₋₆ alkylene groups within L¹, L², or L³ is optionally substituted with (L⁴)ₒ-Y³;
each R^{N} is independently selected from H, C₁₋₃ alkyl, C₁₋₃ haloalkyl, and (L⁴)ₒ-Y³;
each L⁴ is independently selected from N(R^{N1}), O, C(═O), S(═O)₂, C₁₋₆ alkylene, —(OCH₂CH₂)ₓ—, and —(CH₂CH₂O)ₓ—;
each R^{N1} is independently selected from H, C₁₋₃ alkyl, and C₁₋₃ haloalkyl;
p is an integer from 1 to 20;
o is an integer from 1 to 10;
each x is independently an integer from 1 to 2,000;
each Y³ is independently selected from a moiety of formula (i) and a moiety of formula (ii):

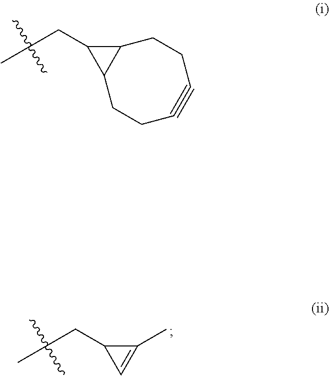

Y¹ is selected from NR^{c1}R^{1A}, OR², and C(═O)R³;
R^{1A} selected from H, an amine protecting group, and a fluorophore;
R² is selected from H, an alcohol protecting group, and a fluorophore;
R³ is selected from OR^{a1} and a fluorophore;
Y² is selected from C(═O)OR^{a1}, NR^{c1}R⁴, OR⁵; and a group reactive with a side chain of an amino acid of a protein;
R^{a1} is selected from H and a carboxylic acid protecting group;
R^{c1} is selected from H, C₁₋₃ alkyl, and C₁₋₃ haloalkyl;
R⁴ is selected from H and an amine protecting group; and
R⁵ is selected from H and an alcohol protecting group.

In some embodiments, R¹ is H.

In some embodiments, n is an integer from 1 to 5, and each L¹ is selected from NH, O, C(═O), and C₁₋₆ alkylene.

In some embodiments, m is an integer from 1 to 5, and each L² is independently selected from NH, C(═O), C₁₋₆ alkylene, —(OCH₂CH₂)ₓ—, and —(CH₂CH₂O)ₓ—.

In some embodiments, m is 5.

In some embodiments, x is an integer from 1 to 10.

In some embodiments:
R¹ is H;
n is an integer from 1 to 5, and each L¹ is selected from NH, O, C(═O), and C₁₋₆ alkylene;
m is an integer from 1 to 5, and each L² is independently selected from NH, O, C(═O), C₁₋₆ alkylene, —(OCH₂CH₂)ₓ—, and —(CH₂CH₂O)ₓ—; and
x is an integer from 2 to 10.

In some embodiments:
p is an integer from 1 to 5, each L³ is selected from NR^{N}, O, C(═O), and C₁₋₆ alkylene, said C₁₋₆ alkylene is optionally substituted with (L⁴)ₒ-Y³; and
R^{N} is selected from H, C₁₋₃ alkyl, and (L⁴)ₒ-Y³.

In some embodiments, each L³ is selected from NH, O, C(═O), and C₁₋₆ alkylene, which is optionally substituted with (L⁴)ₒ-Y³.

In some embodiments, the compound has formula:

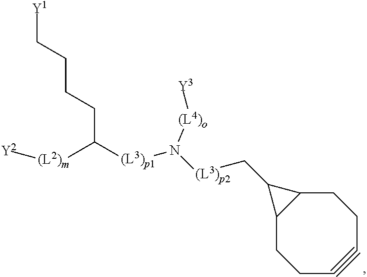

or a pharmaceutically acceptable salt thereof, wherein the sum of p1 and p2 is less than p by at least 1.

In some embodiments:
each L³ is selected from NR^{N}, O, C(═O), and C₁₋₆ alkylene, and
R^{N} is selected from H, C₁₋₃ alkyl, and (L⁴)₀-Y³.

In some embodiments, the compound has formula:

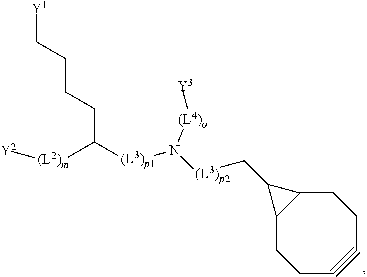

or a pharmaceutically acceptable salt thereof, wherein the sum of p1 and p2 is less than p by at least 1.

In some embodiments, o is an integer from 1 to 5 and each L⁴ is independently selected from NH, O, C(═O), and C₁₋₆ alkylene.

In some embodiments, Y³ is a moiety of formula (i):

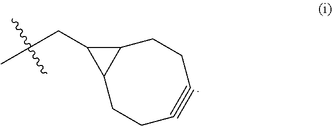 [SMILES: *CC1C2CCC#CCCC12]

In some embodiments, Y³ is a moiety of formula (ii):

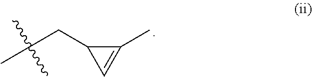 [SMILES: *CC1C=C1C]

In some embodiments, each L³ is selected from NR^{N}, O, C(═O), and C₁₋₆ alkylene.

In some embodiments:
n is 1 and L¹ is C₁₋₆ alkylene;
m is 4, and each L² is independently selected from NH, C(═O), C₁₋₆ alkylene, —(OCH₂CH₂)ₓ—, and —(CH₂CH₂O)ₓ—; and
p is 3, and each L³ is independently selected from NH, O, and C(═O).

In some embodiments, Formula (I) has formula:

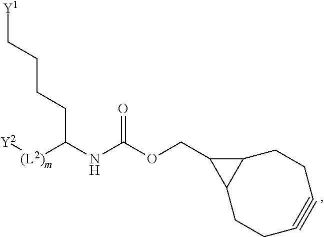

or a pharmaceutically acceptable salt thereof.

In some embodiments, Formula (I) has formula:

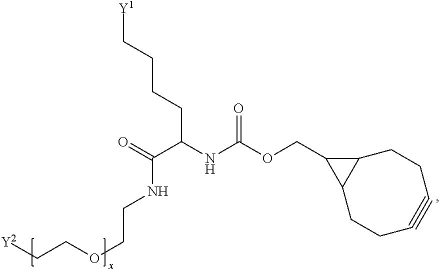

or a pharmaceutically acceptable salt thereof.

In some embodiments:
Y¹ is NHR^{1A}; and
Y² is selected from C(═O)OR^{a1} and a group reactive with a side chain of an amino acid of a protein.

In some embodiments:
Y¹ is NH₂; and
Y² is C(═O)OH.

In some embodiments:
Y¹ is NHR^{1A};
R¹ is an amine protecting group; and
Y² is C(═O)OH.

In some embodiments:
Y¹ is NHR^{1A};
R¹ is a fluorophore; and
Y² is C(═O)OH.

In some embodiments:
Y¹ is NHR^{1A};
R¹ is a fluorophore;
Y² is C(═O)OR^{a1}; and
R^{a1} is a carboxylic acid protecting group.

In some embodiments:
Y¹ is NHR^{1A};
R¹ is a fluorophore; and
Y² is a group reactive with a side chain of an amino acid of a protein.

In some embodiments, the compound of Formula (I) is selected from any one of the following compounds:

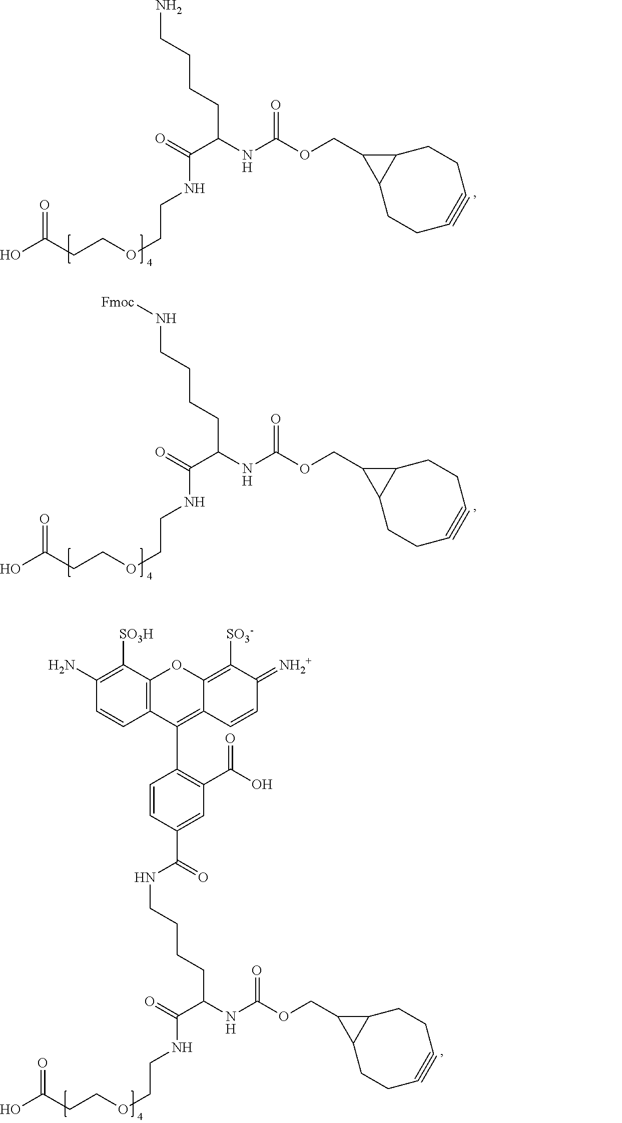

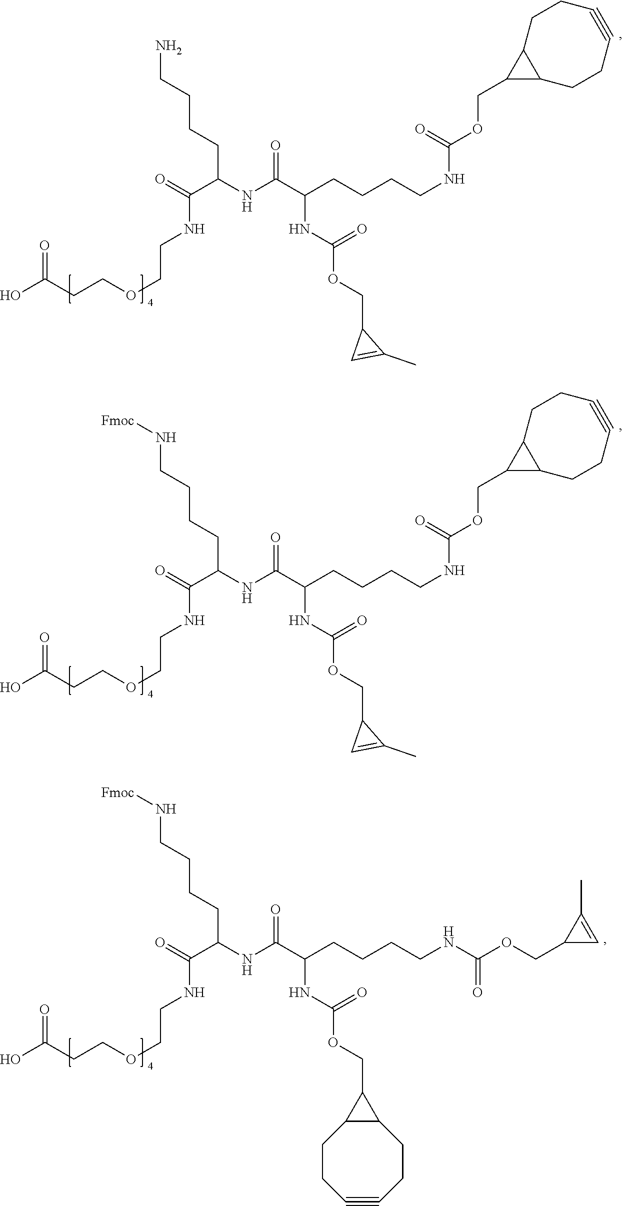

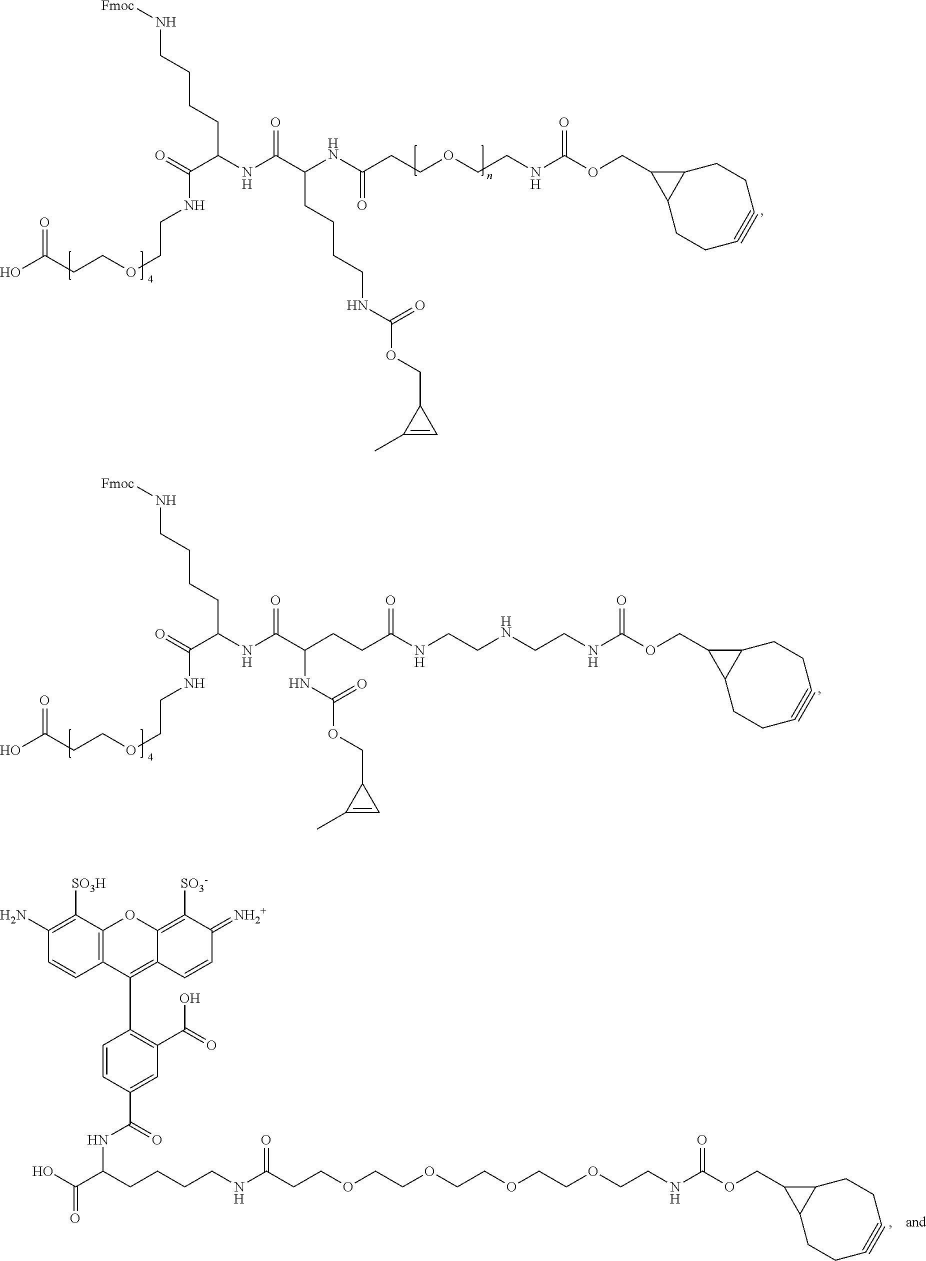

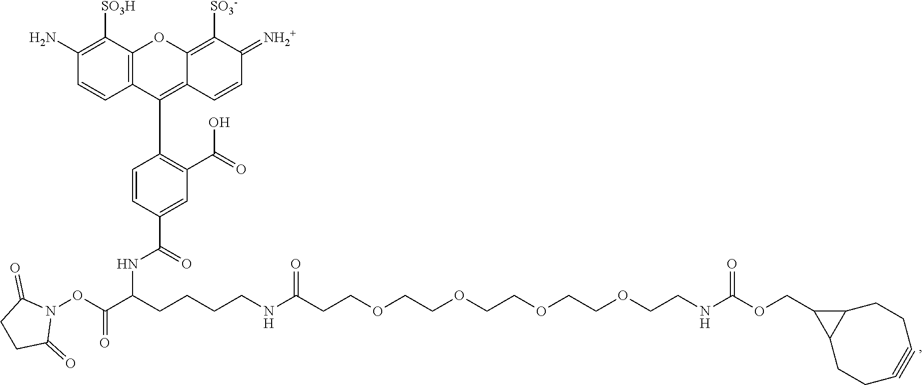

or a pharmaceutically acceptable salt thereof.

In another general aspect, the present disclosure provides a protein conjugate of Formula (II):

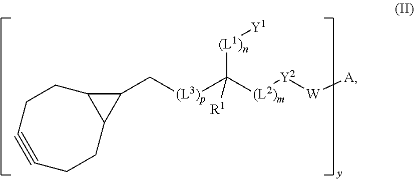

or a pharmaceutically acceptable salt thereof, wherein:
A is a protein;
y is an integer from 1 to 10;
R¹ is selected from H, C₁₋₃ alkyl, and C₁₋₃ haloalkyl;
each L¹ is independently selected from N(R^{N}), O, C(═O), S(═O)₂, C₁₋₆ alkylene, —(OCH₂CH₂)ₓ—, and —(CH₂CH₂O)ₓ—;
n is an integer from 1 to 10;
each L² is independently selected from N(R^{N}), O, C(═O), S(═O)₂, C₁₋₆ alkylene, —(OCH₂CH₂)ₓ—, and —(CH₂CH₂O)ₓ—;
m is an integer from 1 to 10;
each L³ is independently selected from N(R^{N}), O, C(═O), S(═O)₂, C₁₋₆ alkylene, —(OCH₂CH₂)ₓ—, and —(CH₂CH₂O)ₓ—;
wherein each of said C₁₋₆ alkylene groups within L¹, L², or L³ is optionally substituted with (L⁴)ₒ-Y³;
each R^{N} is independently selected from H, C₁₋₃ alkyl, C₁₋₃ haloalkyl, and (L⁴)ₒ-Y³;
each L⁴ is independently selected from N(R^{N1}), O, C(═O), S(═O)₂, C₁₋₆ alkylene, —(OCH₂CH₂)ₓ—, and —(CH₂CH₂O)ₓ—;
each R^{N1} is independently selected from H, C₁₋₃ alkyl, and C₁₋₃ haloalkyl;
p is an integer from 1 to 20;
o is an integer from 1 to 10;
each x is independently an integer from 1 to 2,000;
each Y³ is independently selected from a moiety of formula (i) and a moiety of formula (ii):

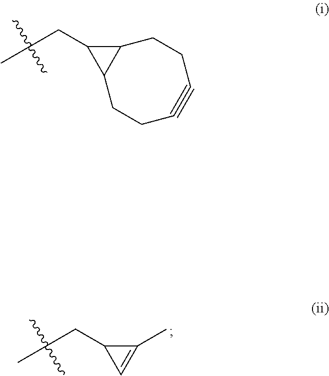

Y¹ is selected from NR^{c1}R^{1A}, OR², and C(═O)R³;
R^{1A}, R², and R³ are each independently selected from a fluorophore;
R^{c1} is selected from H, C₁₋₃ alkyl, and C₁₋₃ haloalkyl;
each W is selected from:
(i) O of a side chain of serine, threonine, or tyrosine of the protein A;
(ii) S of a side chain of cysteine of the protein A;
(iii) NH of a side chain of lysine of the protein A; and
(iv) C(═O) of a side chain of aspartic acid or glutamic acid of the protein A; and
Y² is a residue of a group which, prior to conjugation with the protein A, was a group reactive with a side chain of an amino acid of the protein A.

In some embodiments, the protein is selected from an antibody, an antibody fragment, an engineered antibody, a peptide, and an aptamer.

In some embodiments, the antibody is specific to an antigen which is a biomarker of a disease or condition.

In some embodiments, the disease or condition is cancer.

In some embodiments, y is an integer from 4 to 6.

In some embodiments, each Y² is C(═O) and at least one W is NH of a side chain of lysine of the protein A.

In some embodiments, each Y² is C(═O) and at least one W is S of a side chain of cysteine of the protein A.

In some embodiments, Y¹ is NHR^{1A}.

In some embodiments, Y¹ is OR².

In some embodiments, Y¹ is C(═O)R³.

In some embodiments, R¹ is H.

In some embodiments, n is an integer from 1 to 5, and each L¹ is selected from NH, O, C(═O), and C₁₋₆ alkylene.

In some embodiments, m is an integer from 1 to 5, and each L² is independently selected from NH, C(═O), C₁₋₆ alkylene, —(OCH₂CH₂)ₓ—, and —(CH₂CH₂O)ₓ—.

In some embodiments, m is 5.

In some embodiments, x is an integer from 1 to 10.

In some embodiments:
R¹ is H;
n is an integer from 1 to 5, and each L¹ is selected from NH, O, C(═O), and C₁₋₆ alkylene;
m is an integer from 1 to 5, and each L² is independently selected from NH, O, C(═O), C₁₋₆ alkylene, —(OCH₂CH₂)ₓ—, and —(CH₂CH₂O)ₓ—; and
x is an integer from 2 to 10.

In some embodiments:
p is an integer from 1 to 5,
each L³ is selected from NR^{N}, O, C(═O), and C₁₋₆ alkylene,
said C₁₋₆ alkylene is optionally substituted with (L⁴)ₒ-Y³; and
R^{N} is selected from H, C₁₋₃ alkyl, and (L⁴)₀-Y³.

In some embodiments, each L³ is selected from NH, O, C(═O), and C₁₋₆ alkylene, which is optionally substituted with (L⁴)ₒ-Y³.

In some embodiments, the conjugate has formula:

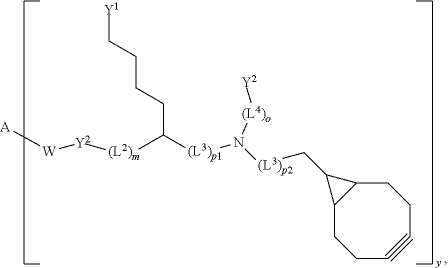

or a pharmaceutically acceptable salt thereof, wherein the sum of p1 and p2 is less than p by at least 1.

In some embodiments:
each L³ is selected from NR^{N}, O, C(═O), and C₁₋₆ alkylene, and
R^{N} is selected from H, C₁₋₃ alkyl, and (L⁴)₀-Y³.

In some embodiments, the conjugate has formula:

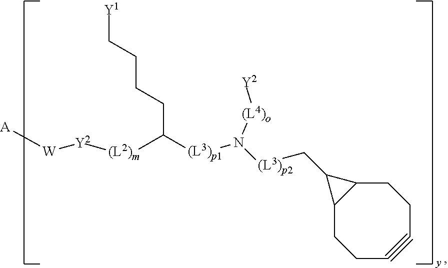

or a pharmaceutically acceptable salt thereof, wherein the sum of p1 and p2 is less than p by at least 1.

In some embodiments, o is an integer from 1 to 5 and each L⁴ is independently selected from NH, O, C(═O), and C₁₋₆ alkylene.

In some embodiments, Y³ is a moiety of formula (i):

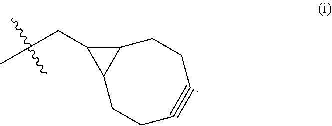 [SMILES: *CC1C2CCC#CCCC12]

In some embodiments, Y³ is a moiety of formula (ii):

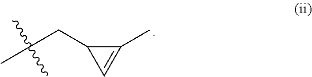 [SMILES: *CC1C=C1C]

In some embodiments, each L³ is selected from NR^{N}, O, C(═O), and C₁₋₆ alkylene.

In some embodiments:
n is 1 and L¹ is C₁₋₆ alkylene;
m is 4, and each L² is independently selected from NH, C(═O), C₁₋₆ alkylene, —(OCH₂CH₂)ₓ—, and —(CH₂CH₂O)ₓ—; and
p is 3, and each L³ is independently selected from NH, O, and C(═O).

In some embodiments, the conjugate of Formula (I) has formula:

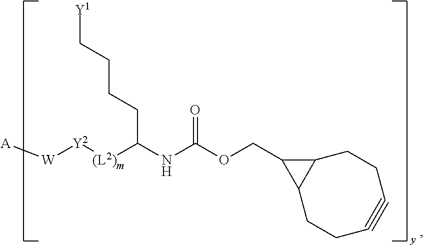

or a pharmaceutically acceptable salt thereof.

In some embodiments, the conjugate of Formula (I) has formula:

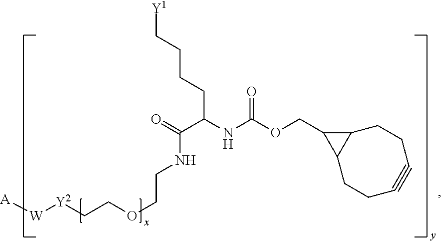

or a pharmaceutically acceptable salt thereof.

In yet another general aspect, the present disclosure provides a composition comprising the conjugate as described herein, or a pharmaceutically acceptable salt thereof, and an inert carrier.

In some embodiments, the composition is an aqueous solution.

In yet another general aspect, the present disclosure provides a method of examining a cell or a component of a cell, the method comprising:
(i) contacting the cell with a conjugate as described herein comprising the fluorophore, or a pharmaceutically acceptable salt thereof, or a composition containing the conjugate as described herein;
(ii) imaging the cell with an imaging technique; and
(iii) after (ii), contacting the cell with a compound of Formula (III):

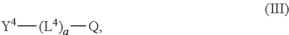

or a pharmaceutically acceptable salt thereof, wherein:
Y⁴ is selected from N₃ and a moiety of formula (iii):

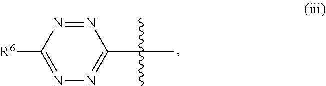

R⁶ is selected from H, C₁₋₆ alkyl, and C₁₋₆ haloalkyl, wherein said C₁₋₆ alkyl is optionally substituted with OH, NH₂, or COOH;
each L⁴ is independently selected from N(R^{N}), O, C(═O), S(═O)₂, C₁₋₆ alkylene, C₆₋₁₀ arylene, C₆₋₁₀ perfluoroarylene, —(OCH₂CH₂)ₓ—, and —(CH₂CH₂O)ₓ—;
a is an integer from 1 to 10;
each R^{N} is selected from H and C₁₋₃ alkyl;
x is an integer from 1 to 2,000; and
Q is a quencher,
wherein the contacting of step (iii) results in decrease of the fluorescence of the fluorophore in the conjugate of Formula (II), or a pharmaceutically acceptable salt thereof.

In some embodiments, the imaging technique is a fluorescence imaging.

In some embodiments, Y⁴ is N₃.

In some embodiments, the compound of Formula (III) has formula:

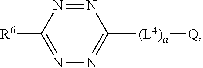

or a pharmaceutically acceptable salt thereof.

In some embodiments, R⁶ is H.

In some embodiments, R⁶ is C₁₋₆ alkyl, optionally substituted with OH, NH₂, or COOH.

In some embodiments, a is an integer from 1 to 7, and each L⁴ is independently selected from NH, C(═O), C₁₋₆ alkylene, C₆₋₁₀ arylene, C₆₋₁₀ perfluoroarylene, and —(CH₂CH₂O)ₓ—.

In some embodiments, the compound of Formula (III) is selected from any one of the following compounds:

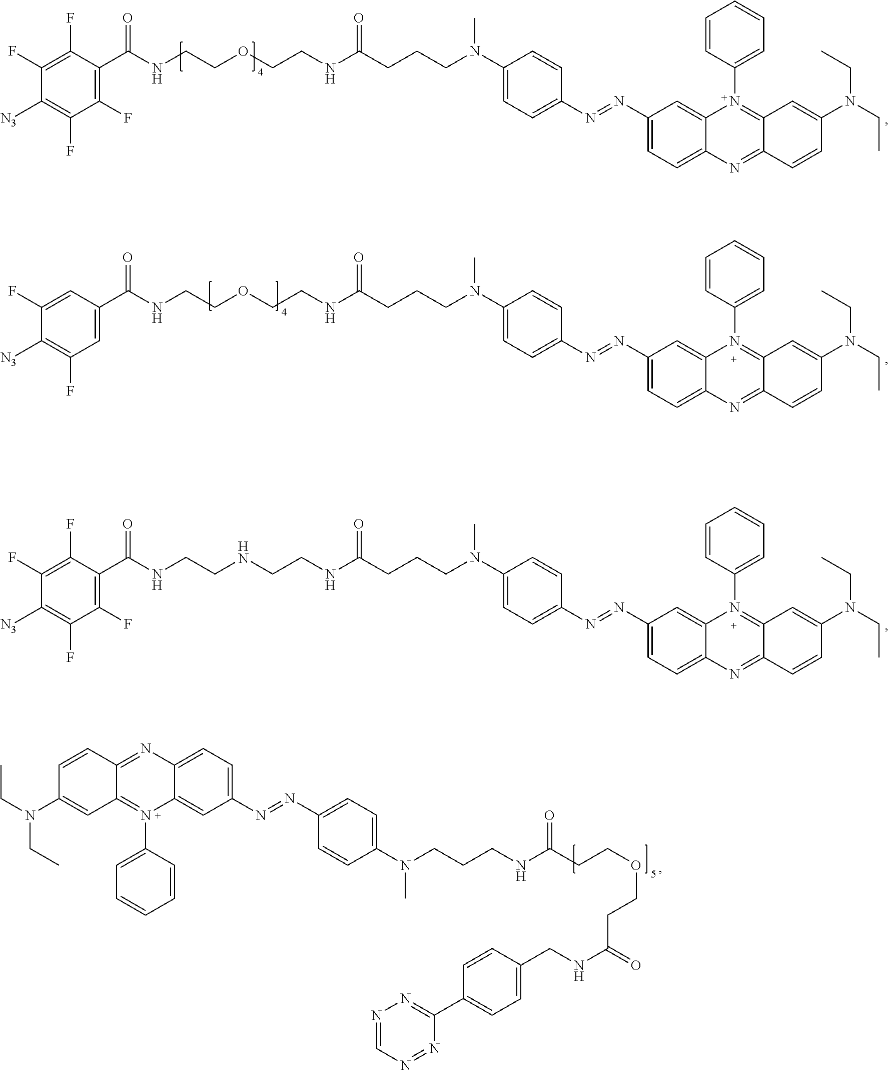

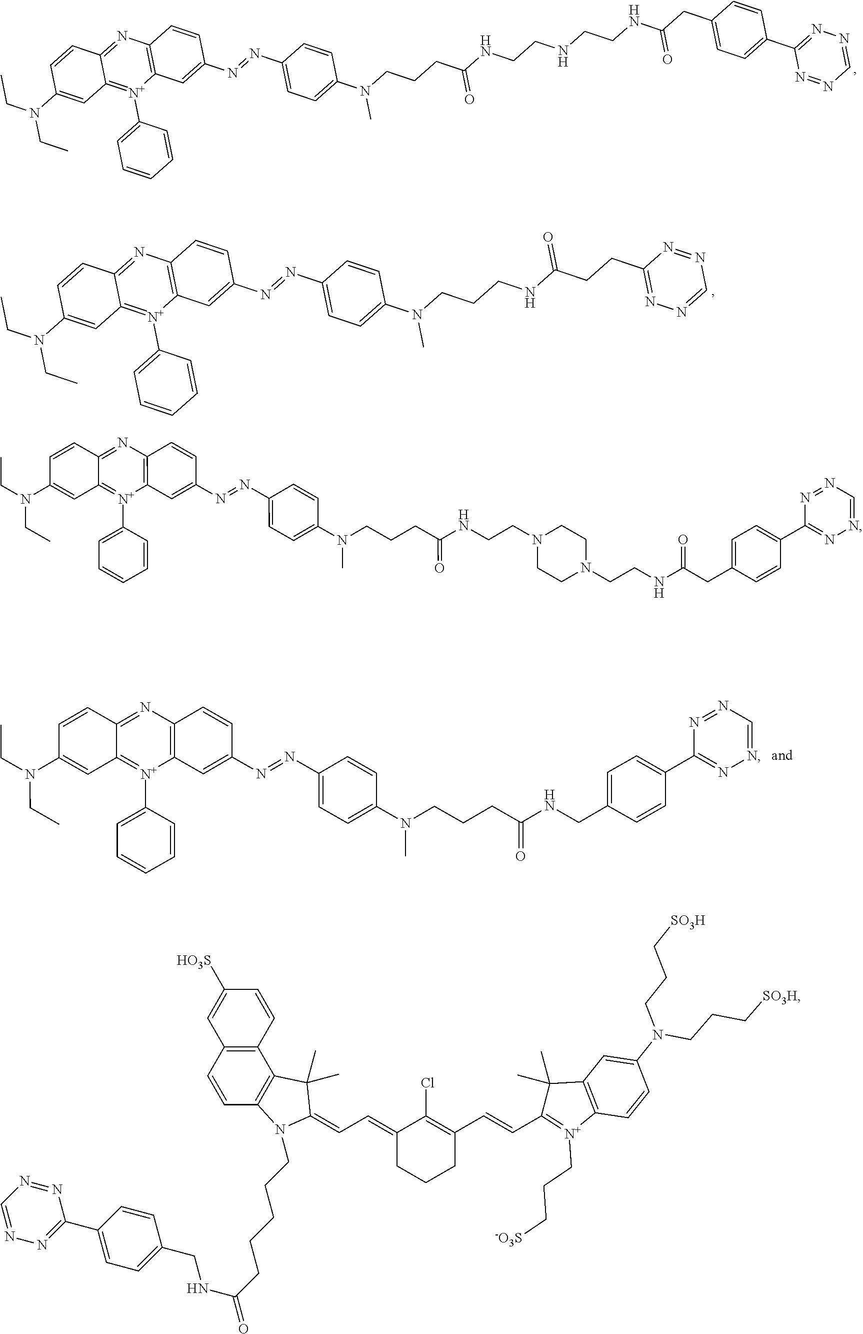

or a pharmaceutically acceptable salt thereof.

In yet another general aspect, the present disclosure provides a method selected from:
profiling a cell;
examining a cell using a cytometry technique;
diagnosing a disease or condition of a subject by examining pathology of a cell obtained from the subject;
monitoring progression of disease or condition of a subject by examining pathology of a cell obtained from the subject; and
detecting a disease biomarker in a cell;
the method comprising:
(i) obtaining a cell from the subject; and
(ii) examining the cell according to the method as described herein.

In some embodiments, the cell is obtained from the subject using image-guided biopsy, fine needle aspiration (FNA), surgical tissue harvesting, punch biopsy, liquid biopsy, brushing, swab, touch-prep, fluid aspiration or blood analysis.

In some embodiments, the cytometry technique is selected from image cytometry, holographic cytometry, Fourier ptychography cytometry, and fluorescence cytometry.

In some embodiments, the cell is selected from a cancer cell, an immune system cell, and a host cell.

In some embodiments, the disease or condition is cancer.

In some embodiments, the cancer is selected from lymphoma, breast cancer, skin cancer, lymphoma nodes, head and neck cancer, and oral cancer.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 contains chemical structures of FAST linkers containing rTCO, TCO, CP, cTCO, dTCO, and BCN.

FIG. 2 contains line plot showing that the dynamic signal of reactive fluorophore-TCO probes (FAST probes, see Example 1) can be used to interrogate their own chemical stability across contexts, whether as the free dye, in the setting of a labeled antibody, or on the surface of cells. Comparing the extent of quenching after environmental exposure (t₁) to the quenching at baseline (t₀) enables quantification of dienophile (e.g., TCO) reactivity, a metric of stability.

FIG. 3 contains a bar graph showing quenching-based quantification of dienophile (e.g., xTCO) survival (10 nM solution in PBS) after 2 hours of exposure to ambient light reveals structure-dependent loss of Tz reactivity, with rTCO much more stable than TCO or dTCO.

FIG. 4A shows comparison of rTCO survival on the benchtop shielded by foil (dark) or exposed to ambient light. Mechanistic studies reveal that increasing the total fluorophore concentration in solution does not influence survival, suggesting an intramolecular mechanism, nor does the total probe concentration within the studied nanomolar range.

FIG. 4B shows that rTCO survival increases dramatically under an argon atmosphere, indicating a role for air. Also, addition of 10 μM Trolox as a scavenger of reactive oxygen species also enhances rTCO stability/survival.

FIG. 5 shows oxygen-mediated degradation. Serial LCMS analysis of a dTCO probe exposed to light and air and then concentrated for analysis reveals formation of multiple degradation products. Mass fragments (+16, +32) are consistent with oxygen adducts.

FIG. 6A shows reactivity, stability, and quenching performance of dienophiles, including CP and BCN. Probes were synthesized with bicyclononyne (BCN) and cyclopropene (CP) dienophiles and assessed for their survival under ambient light exposure. Inset: relationship between second order TCO/Tz rate constant and degradation.

FIG. 6B shows relative quenching efficiency of the intact probes with BHQ3-Tz, which contains a flexible PEG5 linker between the BHQ3 and tetrazine, tested immediately after dilution into PBS (no light/02 exposure). Quenching performance varies significantly as a function of dienophile. BCN and rTCO exhibit the best performance (lowest residual fluorescence), while CP is intermediate; cTCO, TCO, and dTCO have the highest residual signal.

FIG. 7A shows kinetic and biological performance of BCN- and CP-based probes. Analytical kinetics for the reaction of cetuximab labeled with BCN/CP-AF647 probes. Intensity vs time profiles after addition of BHQ3-Tz reveal about 1400-fold acceleration of the quenching reaction vs the predicted kinetics for the Tz/BCN reaction alone, with an effective second order rate constant of 4.5×10⁶ M⁻¹ s⁻¹. A 500× acceleration is observed for the CP probe, which translates the expected time for complete reaction at the experimental concentration from months to hours.

FIG. 7B contains an image and a bar graph showing that signal dynamics are equally fast in the cellular context, with complete quenching of the cetuximab staining observed within two minutes after addition of BHQ3-Tz (1 μM) (see FIG. 7A), consistent with the calculated impact on kinetics. At 10 μM BHQ3-Tz, quenching for the CP probe that would be expected to take 36 hours can be accelerated to 2-3 minutes.

FIG. 7C contains images showing that quenching of the CP probe after extended photoexposure reveals complete signal elimination even after 2 minutes of continuous high-intensity illumination, with >95% quenching evident in both qualitative signal dynamics and the quantitative intensity profile.

FIG. 8A shows chemical synthesis of an azido-tetrafluorobenzene BHQ3 click-quencher (6).

FIG. 8B shows universal click acceleration—BCN quenching with Azide-BHQ3. Kinetic profiling revealed a biphasic process with an effective rate constant for the slow component of 6700 M⁻¹s⁻¹, consistent with >5000-fold acceleration. This change in the underlying azide-alkyne reaction kinetics is sufficient to translate the reaction timeframe from one week to two minutes at routine imaging conditions (10 μM azide-BHQ3).

FIG. 8C contains images showing that the signal from cetuximab-BCN-AF647 is completely removed in 2 minutes at just 10 μM azide-BHQ3 quencher concentrations, matching the predicted kinetics measured in vitro.

FIG. 9 shows that CP and BCN probe quenching is rapid on the surface of cells. The timecourse of signal elimination is captured after addition of the BHQ3-Tz quencher to cells stained with cetuximab-CP-AF647 and after addition of azide-BHQ3 to cells stained with cetuximab-BCN-AF647. Cells were incubated with the quencher for the indicated time and then rinsed three times prior to imaging to exclude any non-specific signal reduction due to the quencher in solution. Each vertical pair thus represents an independent experiment. The fluorescent signal is substantially eliminated after 1 minute of reaction time and further reduced with extended incubation times.

FIG. 10A contains line plots showing rTCO quenching as a function of time exposed to light and air, plotting signal vs time before and after addition of BHQ3-Tz; the two traces in each graph are independent replicates.

FIG. 10B contains line plot showing that fluorophore intensity is stable vs time in solution exposed to light and air under the conditions used for FIG. 1-FIG. 6, indicating that quenching signal dynamics are not related to an alteration in the dye.

FIG. 10C contains a line plot showing that the signal intensity of an AF488 control fluorophore (without embedded TCO/dienophile) is not affected by the addition of the BHQ3-Tz quencher.

FIG. 10D shows quantitative stability of dTCO-AF488 exposed to light and air and the impact of added trolox as a function of concentration during either 45 min or 2 h of exposure to light and air.

FIG. 10E shows line plot showing that BCN stability is enhanced by addition of Trolox, matching the observed behavior of rTCO.

FIG. 11 shows the results of probe quenching by 20 μM Tz-BHQ3 on the surface of cells stained with cetuximab-CP-AF488, cetuximab-BCN-AF488, and cetuximab-rTCO-AF488. BCN probe showed superior stability under microscope compared to TCO and rTCO probes.

FIG. 12A contains line plot showing dramatically enhanced reaction rate for the reaction of cetuximab-BCN-AF467 with azide-BHQ3 and BHQ3-Tz compared to the predicted values. The absolute rate of the tetrazine reaction is faster compared to the azide reaction.

FIG. 13 is an overview diagram with clinical needs and turnaround times. Scant cells can be obtained by fine needle aspiration (FNA), brushings, touch preps or blood/fluid samples. Essential to the integrated and automated processing of such cells are cycling methods, instrumentation and computational approaches. Indeed the analysis relies heavily on deep learning and AI approaches to extract information from dozens of channels and convert them into a medical diagnosis. For point-of-care settings, all of the above occur within reasonable time frames and at low cost. DL, deep learning; AI, artificial intelligence.

FIG. 14 is a table containing overview of some experimental (top) and commercial systems (bottom).

FIG. 15 contains schemes and images showing cyclic labeling technologies for multiplexed assessment of cancer and host cell markers; different cycling techniques and an example of immune cell profiling in FNA sample using cell based cycling.

FIG. 16 contains a structural scheme of a miniscope. A finger-sized, single-channel fluorescent microscope is structured like a conventional fluorescent microscope but uses an LED as an excitation source and a gradient refractive index (GRIN) lens as an objective.

FIG. 17 is an image of Cytometry Portable Analyzer (CytoPAN). The system is integrates five light sources and a quad-band filter. No mechanical parts are necessary for multiple channel imaging.

FIG. 18 is an image of the analysis of an FNA specimen from a breast cancer patient. Cancer cells were identified through the staining of QUAD markers: EGFR, EpCAM, HER2, MUC1 or EGFR, EpCAM, CK, MUC1. Immune cells through CD45 staining. Images were taken at 5× magnification.

FIG. 19 shows that CytoPAN software automatically profiles individual cells in multi-color channels and generates a summary report to guide cancer diagnosis.

FIG. 20 is a table showing comparison of some cellular cycling techniques. The table provides an overview of three recently developed technologies: ABCD, SCANT and the methods and compounds of the present application (FAST). Collectively, the technologies allow imaging of 20-40 targets in each individual cells and this can be used for cellular mapping (e.g. immune cell profiling), cellular pathway analysis or heterogeneity studies.

### DETAILED DESCRIPTION

Molecular analyses of cancer cells are essential in establishing diagnosis and guiding available treatments. In an ideal world, one would like to harvest cancers frequently and in the least invasive manner so that molecular information can be obtained periodically through treatment and cancer evolution. “Liquid biopsies”, i.e., the interrogation of circulating tumor cells, extracellular vesicles or cell-free DNA in the peripheral blood, provide one such option, but detection of actionable events is rare and overall sensitivities can be low. More importantly, circulating tumor diagnostics cannot currently be traced back to their anatomical origin, whether primary tumor or metastatic site. This limits the ability to correlate molecular events with radiographic/imaging measures of cancer behavior, invasiveness, and progression.

An alternative method is fine needle aspiration (FNA) that yields cells rather than tissue from a tumor, are inherently of known localization, and which can be processed expeditiously, i.e. do not require embedding or sectioning. FNA are obtained with small gauge needles (20-25 G) and are generally well tolerated. As such, image guided FNA are ideally suited for repeat sampling and have a very low risk of procedural complications. However, as mentioned previously, the challenge in processing these cellular samples is that they can be scant (often <1,000 cells per pass), limiting the number of special stains that can be done, and also delicate, lacking the structural scaffold of intact tissue architecture. Even when processed with fluorescent antibodies, the number of different stains is practically limited to 4-6 and often not sufficient for in depth cancer cell profiling for diagnosis or treatment assessment. This limitation also extends to immune profiling, where significantly more than 4-6 markers need to be interrogated so that analysis reflects the representative immunocyte populations in the tumor microenvironment. In contrast, single cell cycling methods of the present disclosure allow repeat staining, destaining, and re-staining of harvested cellular samples for better therapy assessment in both cancer cells and host immune cells.

Most fluorescent cycling methods were originally developed for paraffin embedded tissue sections that can withstand harsh destaining/quenching conditions. Unfortunately, these harsh conditions typically require oxidants for bleaching at strongly alkaline pH (e.g., 4.5% H₂O₂, 24 mM NaOH, pH>12) and are not well suited for cellular FNA samples. Furthermore, it is not uncommon for other antibody-DNA cycling technologies to require a significant investment in nucleic acid tags/technologies and take hours-days of sample processing, including ABCD and SCANT. Similar technical hurdles accompany other conventional methods for antibody-DNA based imaging, including intricate chemical steps for DNA barcode activation and antibody-DNA bioconjugation, and/or complex fluidics required for cycling multiple sequential staining solutions.

As described more fully below, the present disclosure provides fast and gentle reagents and methods of single-cell cycling. In one embodiment, the disclosure provides ultra-fast BCN-based clickable fluorophores (FAST probes).

### Reagents and Linkers

In some embodiments, the present disclosure provides a tridentate reagent comprising a bicyclononyne (BCN)-based click-reactive group capable of undergoing a click reaction with a tetrazine (Tz) or an azide (N₃) reagent comprising a fluorescence quencher, a fluorophore capable of being detected by fluorescent imaging, and a group reactive with a side chain of an amino acid of a protein. The tridentate reagent may be used to covalently modify a side chain of at least one amino acid of the protein. Hence, the covalently modified protein comprises a fluorophore (which makes the protein detectable by fluorescent imaging) and a BCN reactive group capable of undergoing a reaction with a tetrazine (Tz) or azide reagent comprising a fluorescence quencher. The tridentate reagent may be used to covalently modify a protein simultaneously with a fluorophore and a fluorescent quencher, thereby rendering the protein undetectable by fluorescence imaging (the quencher absorbs the fluorescence from the fluorophore).

In some embodiments, the tridentate reagent, as well as the synthetic intermediates useful in preparing the tridentate reagent, are encompassed by the Formula (I):

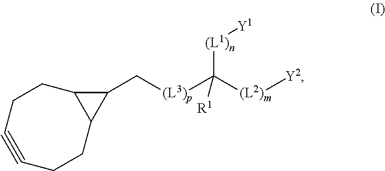

or a pharmaceutically acceptable salt thereof, wherein:
R¹ is selected from H, C₁₋₃ alkyl, and C₁₋₃ haloalkyl;
each L¹ is independently selected from N(R^{N}), O, C(═O), S(═O)₂, C₁₋₆ alkylene, —(OCH₂CH₂)ₓ—, and —(CH₂CH₂O)ₓ—;
n is an integer from 1 to 10;
each L² is independently selected from N(R^{N}), O, C(═O), S(═O)₂, C₁₋₆ alkylene, —(OCH₂CH₂)ₓ—, and —(CH₂CH₂O)ₓ—;
m is an integer from 1 to 10;
each L³ is independently selected from N(R^{N}), O, C(═O), S(═O)₂, C₁₋₆ alkylene, —(OCH₂CH₂)ₓ—, and —(CH₂CH₂O)ₓ—;
wherein each of said C₁₋₆ alkylene groups within L¹, L², or L³ is optionally substituted with (L⁴)ₒ-Y³;
each R^{N} is independently selected from H, C₁₋₃ alkyl, C₁₋₃ haloalkyl, and (L⁴)ₒ-Y³;
each L⁴ is independently selected from N(R^{N}), O, C(═O), S(═O)₂, C₁₋₆ alkylene, —(OCH₂CH₂)ₓ—, and —(CH₂CH₂O)ₓ—;
each R^{N1} is independently selected from H, C₁₋₃ alkyl, and C₁₋₃ haloalkyl;
p is an integer from 1 to 20;
o is an integer from 1 to 10;
each x is independently an integer from 1 to 2,000;
each Y³ is independently selected from a moiety of formula (i) and a moiety of formula (ii):

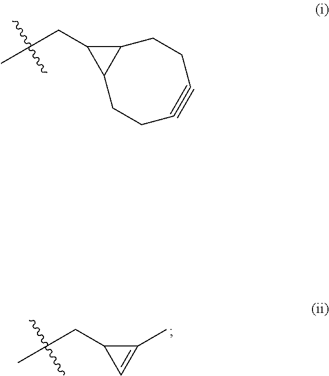

Y¹ is selected from NR^{c1}R^{1A}, OR², and C(═O)R³;
R^{1A} selected from H, an amine protecting group, and a fluorophore;
R² is selected from H, an alcohol protecting group, and a fluorophore;
R³ is selected from OR^{a1} and a fluorophore;
Y² is selected from C(═O)OR^{a1}, NR^{c1}R⁴, OR⁵; and a group reactive with a side chain of an amino acid of a protein;
R^{a1} is selected from H and a carboxylic acid protecting group;
R^{c1} is selected from H, C₁₋₃ alkyl, and C₁₋₃ haloalkyl;
R⁴ is selected from H and an amine protecting group; and
R⁵ is selected from H and an alcohol protecting group.

In some embodiments, R¹ is H. In some embodiments, R¹ is C₁₋₆ alkyl.

In some embodiments, n is an integer from 1 to 7. In some embodiments, n is an integer from 1 to 5. In some embodiments, n is 1. In some embodiments, n is 2. In some embodiments, n is 3.

In some embodiments, each L¹ is independently selected from NH, O, C(═O), C₁₋₆ alkylene, —(OCH₂CH₂)ₓ—, and —(CH₂CH₂O)ₓ—. In some embodiments, n is an integer from 1 to 5, and each L¹ is selected from NH, O, C(═O), and C₁₋₆ alkylene. In some embodiments, n is 1 and L¹ is C₁₋₆ alkylene. In some embodiments, at least one L¹ is N(R^{N}), and R^{N} is (L⁴)ₒ-Y³. In some embodiments, at least one L¹ is C₁₋₆ alkylene substituted with (L⁴)ₒ-Y³.

In some embodiments, m is an integer from 1 to 7. In some embodiments, m is an integer from 1 to 5. In some embodiments, m is at least 1. In some embodiments, m is an integer from 2 to 10. In some embodiments, m is an integer from 3 to 7.

In some embodiments, m is an integer from 1 to 5, and each L² is independently selected from NH, O, C(═O), C₁₋₆ alkylene, —(OCH₂CH₂)ₓ—, and —(CH₂CH₂O)ₓ—. In some embodiments, m is 4, and each L² is independently selected from NH, C(═O), C₁₋₆ alkylene, and —(OCH₂CH₂)ₓ—. In some embodiments, at least one L² is N(R^{N}), and R^{N} is (L⁴)₀-Y³. In some embodiments, at least one L² is C₁₋₆ alkylene substituted with (L⁴)₀-Y³. In some embodiments, m and L² are selected such that the (L²)ₘ is sufficiently long for the BCN moiety within the Formula (I) to not interfere with a function of a protein (e.g., an antibody) which may be attached to Y² as described further herein.

In some embodiments, p is an integer from 1 to 7. In some embodiments, p is an integer from 1 to 5. In some embodiments, p is at least 1. In some embodiments, p is an integer from 2 to 10. In some embodiments, p is an integer from 3 to 7. In some embodiments, p is an integer from 1 to 15. In some embodiments, p is an integer from 1 to 10. In some embodiments, p is an integer from 1 to 7.

In some embodiments, each L³ is independently selected from NH, O, C(═O), C₁₋₆ alkylene, —(OCH₂CH₂)ₓ—, and —(CH₂CH₂O)ₓ—. In some embodiments, p is an integer from 1 to 5, and each L³ is selected from NH, O, C(═O), and C₁₋₆ alkylene. In some embodiments, p is 3 and each L³ is selected from NH, O, and C(═O). In some embodiments, at least one L³ is C₁₋₆ alkylene substituted with (L⁴)₀-Y³. In some embodiments, at least one L³ is N(R^{N}), and R^{N} is (L⁴)₀-Y³.

In some embodiments, o is an integer from 1 to 7. In some embodiments, o is an integer from 1 to 4. In some embodiments, o is an integer from 1 to 3. In some embodiments, o is an integer from 1 to 5. In some embodiments, each L⁴ is independently selected from NH, O, C(═O), and C₁₋₆ alkylene. In some embodiments, each L⁴ is independently selected from NH, O, and C(═O).

In some embodiments, R^{N} is H. In some embodiments, R^{N} IS C₁₋₃ alkyl. In some embodiments, R^{N} is (L⁴)₀-Y³. In some embodiments, R^{N1} is H. In some embodiments, R^{N1} IS C₁₋₃ alkyl.

In some embodiments, x is an integer from 2 to 10. In some embodiments, x is 3, 4, 5, or 6.

In some embodiments of Formula (I):
R¹ is H;
n is an integer from 1 to 5, and each L¹ is selected from NH, O, C(═O), and C₁₋₆ alkylene;
m is an integer from 1 to 5, and each L² is independently selected from NH, O, C(═O), C₁₋₆ alkylene, —(OCH₂CH₂)ₓ—, and —(CH₂CH₂O)ₓ—;
x is an integer from 2 to 10;
p is an integer from 1 to 5;
each L³ is independently selected from N(R^{N}), O, C(═O), and C₁₋₆ alkylene, wherein said C₁₋₆ alkylene is optionally substituted with (L⁴)ₒ-Y³;
each R^{N} is independently selected from H and (L⁴)ₒ-Y³;
o is an integer from 1 to 5; and
each L⁴ is independently selected from NH, O, C(═O), and C₁₋₆ alkylene.

In some embodiments, the compound of Formula (I) comprises one Y³ group.

In some embodiments, p is an integer from 1 to 3, and each L³ is independently selected from NH, O, and C(═O).

In some embodiments, the compound of Formula (I) has formula:

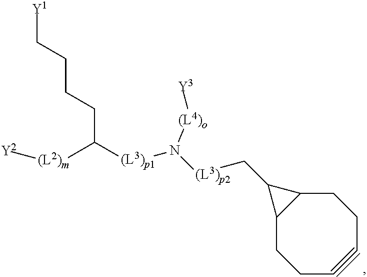

or a pharmaceutically acceptable salt thereof, wherein the sum of p1 and p2 is less than p by at least 1.

In some embodiments, the compound of Formula (I) has formula:

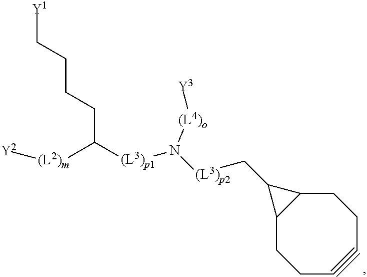

or a pharmaceutically acceptable salt thereof, wherein the sum of p1 and p2 is less than p by at least 1.

In some embodiments, the Y³ (if present) is a moiety of formula (i):

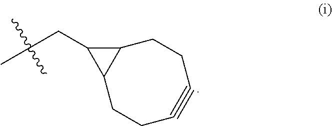 [SMILES: *CC1C2CCC#CCCC12]

In some embodiments, the Y³ (if present) is a moiety of formula (ii):

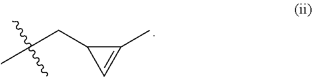 [SMILES: *CC1C=C1C]

In some embodiments of Formula (I):
R¹ is H;
n is 1 and L¹ is C₁₋₆ alkylene;
m is 4, and each L² is independently selected from NH, C(═O), C₁₋₆ alkylene, and —(OCH₂CH₂)ₓ—;
p is 3, and each L³ is independently selected from NH, O, and C(═O); and
x is an integer from 2 to 10.

In some embodiments, the compound of Formula (I) has formula:

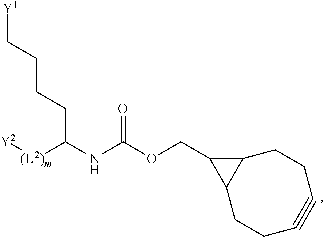

or a pharmaceutically acceptable salt thereof.

In some embodiments, the compound of Formula (I) has formula:

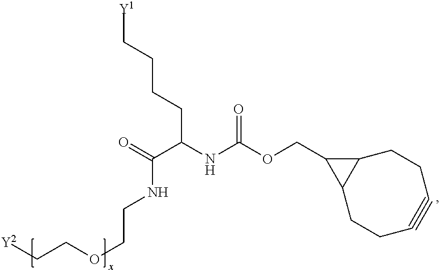

or a pharmaceutically acceptable salt thereof.

In some embodiments, x is selected from 1, 2, 3, 4, 5, 6, 7, 8, 9, and 10. In some embodiments, x is 4 or 5. In some embodiments, x is 4.

In some embodiments, (L¹)ₙ comprises a side chain of an amino acid (e.g., lysine, serine, threonine, cysteine, tyrosine, aspartic acid, or glutamic acid), and Y¹ comprises the terminal functional group of the side chain of the amino acid (e.g., N, O, S, or C(═O)).

In some embodiments, Y¹ is NHR^{1A}.

In some embodiments, R^{1A} is a fluorophore.

In some embodiments, R^{1A} is an amine protecting group.

In some embodiments, Y¹ is NH₂.

In some embodiments, Y¹ is OR².

In some embodiments, Y¹ is OH.

In some embodiments, R² is an alcohol protecting group.

In some embodiments, R² is a fluorophore.

In some embodiments, Y¹ is C(═O)R³.

In some embodiments, Y¹ is C(═O)OH.

In some embodiments, R³ is OR^{a1}, and R^{a1} is a carboxylic acid protecting group.

In some embodiments, R³ is a fluorophore.

In some embodiments, Y² is C(═O)OR^{a1} In some embodiments, Y² is C(═O)OH.

In some embodiments, R^{a1} is a carboxylic acid protecting group.

In some embodiments, Y² is NHR⁴.

In some embodiments, Y² is NH₂.

In some embodiments, R⁴ is an amine-protecting group.

In some embodiments, Y² is OR⁵.

In some embodiments, Y² is OH.

In some embodiments, R⁵ is an alcohol-protecting group.

In some embodiments, Y² is a group reactive with a side chain of an amino acid of a protein. In some embodiments, the group reactive with a side chain of an amino acid of a protein is an activated ester group.

In some embodiments:
Y¹ is NHR^{1A}; and
Y² is selected from C(═O)OR^{a1} and a group reactive with a side chain of an amino acid of a protein.

In some embodiments:
Y¹ is NH₂; and
Y² is C(═O)OH.

In some embodiments:
Y¹ is NHR^{1A};
R¹ is an amine protecting group; and
Y² is C(═O)OH.

In some embodiments:
Y¹ is NHR^{1A};
R¹ is a fluorophore; and
Y² is C(═O)OH.

In some embodiments:
Y¹ is NHR^{1A};
R¹ is a fluorophore;
Y² is C(═O)OR^{a1}; and
R^{a1} is a carboxylic acid protecting group.

In some embodiments:
Y¹ is NHR^{1A};
R¹ is a fluorophore; and
Y² is a group reactive with a side chain of an amino acid of a protein.

In some embodiments, the compound of Formula (I) is selected from any one of the following compounds:

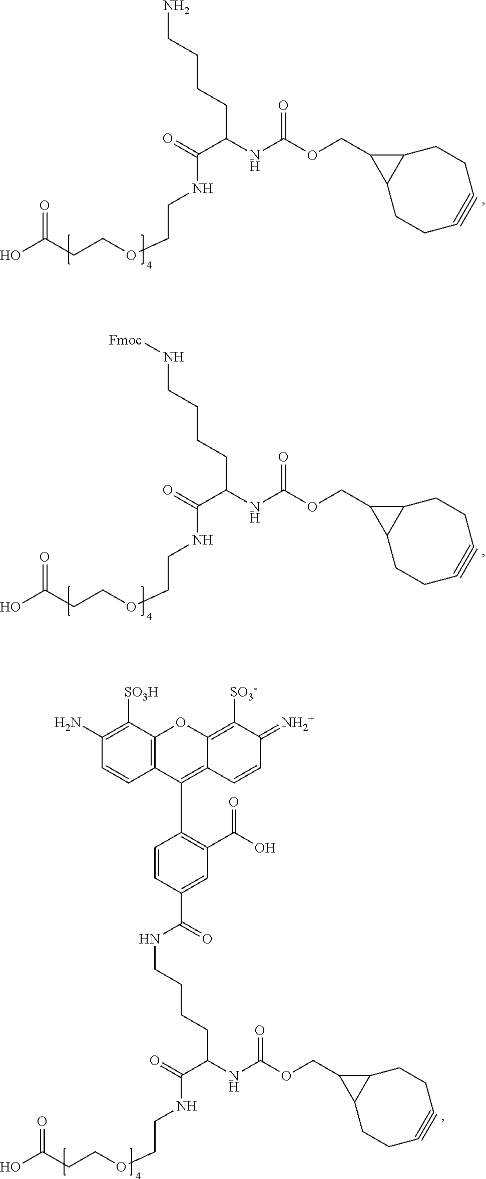

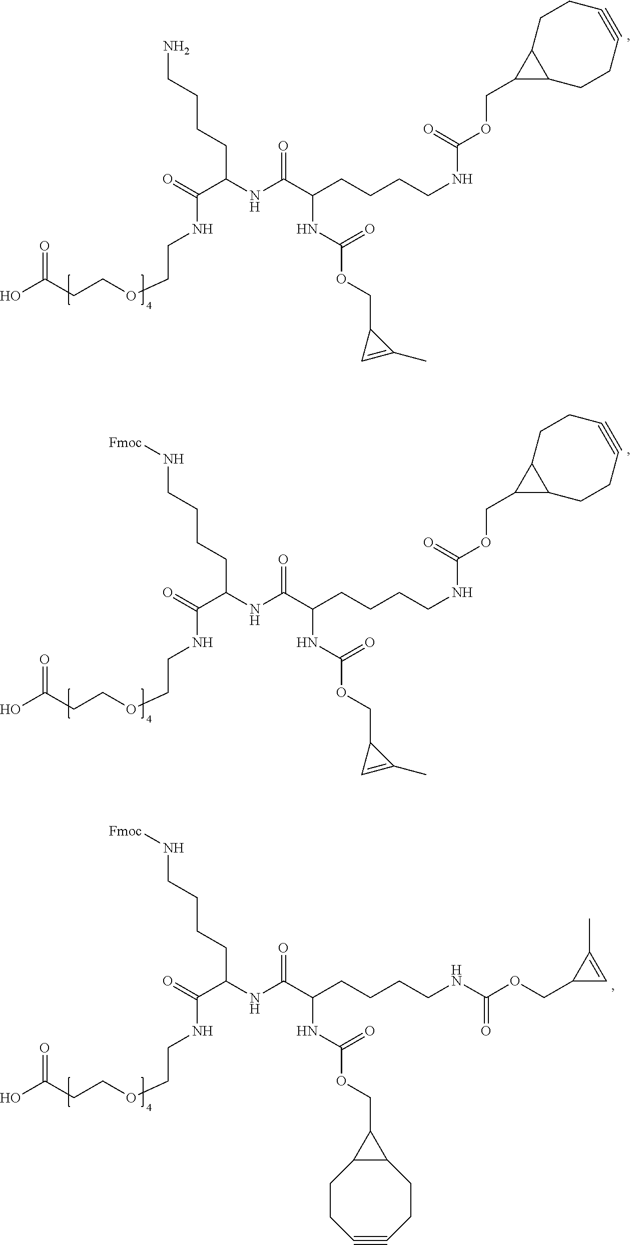

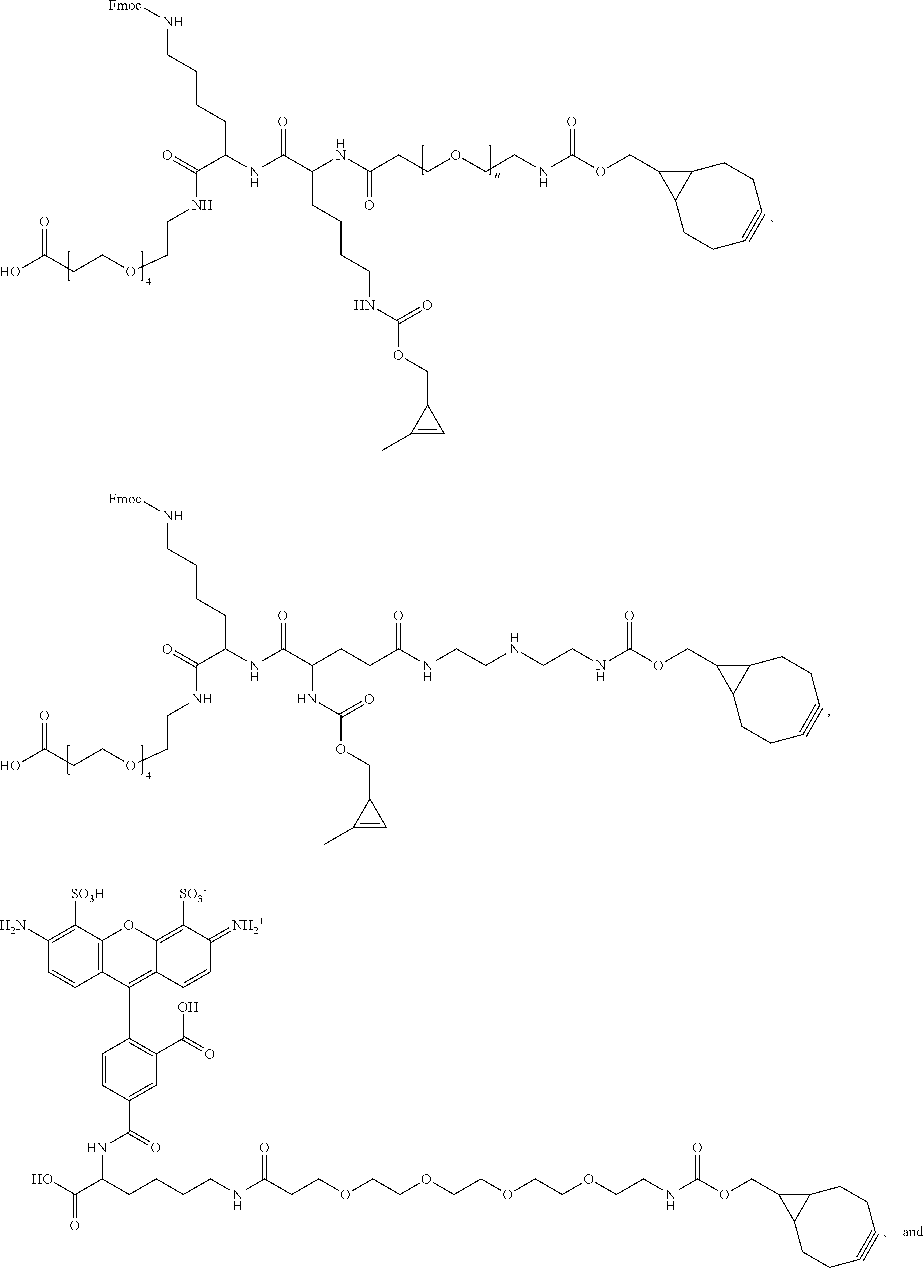

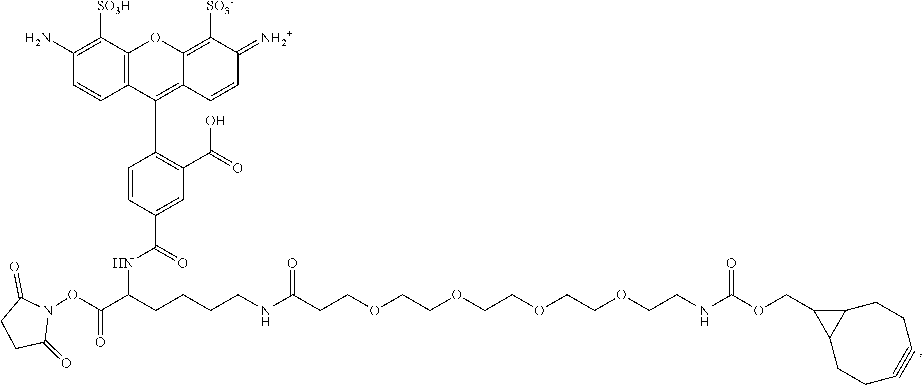

or a pharmaceutically acceptable salt thereof.

In some embodiments, a skilled chemist would be able to select and implement any of the amine protecting groups, alcohol protecting groups, or carboxylic acid protecting groups of the present disclosure. The chemistry of protecting groups can be found, for example, in P. G. M. Wuts and T. W. Greene, Protective Groups in Organic Synthesis, 4^{th} Ed., Wiley & Sons, Inc., New York (2006) (which is incorporated herein by reference), including suitable examples of the protecting groups, and methods for protection and deprotection, and the selection of appropriate protecting groups.

Suitable examples of amine-protecting groups include Carbobenzyloxy (Cbz) group, p-Methoxybenzyl carbonyl (Moz or MeOZ), tert-Butyloxycarbonyl (BOC) group, 9-Fluorenylmethyloxycarbonyl (Fmoc), Acetyl (Ac), Benzoyl (Bz), Benzyl (Bn) group, Carbamate group, p-Methoxybenzyl (PMB), 3,4-Dimethoxybenzyl (DMPM), p-Methoxyphenyl (PMP) group, Tosyl (Ts) group, Troc (trichloroethyl chloroformate), and nosyl group.

Suitable examples of alcohol-protecting groups include acetyl (Ac), benzoyl (Bz), benzyl (Bn), β-methoxyethoxymethyl ether (MEM), dmethoxytrityl, [bis-(4-methoxyphenyl)phenylmethyl](DMT), methoxymethyl ether (MOM), methoxytrityl [(4-methoxyphenyl)diphenylmethyl](MMT), p-methoxybenzyl ether (PMB), methylthiomethyl ether, pivaloyl (Piv), tetrahydropyranyl (THP), tetrahydrofuran (THF), trityl (triphenylmethyl, Tr), silyl ether (most popular ones include trimethylsilyl (TMS), tert-butyldimethylsilyl (TBDMS), tri-iso-propylsilyloxymethyl (TOM), and triisopropylsilyl (TIPS) ethers), methyl ethers, and ethoxyethyl ethers (EE).

Suitable examples of carboxylic acid protecting groups include methyl esters, benzyl esters, tert-butyl esters, esters of 2,6-disubstituted phenols (e.g., 2,6-dimethylphenol, 2,6-diisopropylphenol, 2,6-di-tert-butylphenol), silyl esters, orthoesters, and oxazoline.

Suitable examples of groups reactive with a side chain of an amino acid of a protein are described, for example, in D. Shannon, Covalent protein modification: the current landscape of residue-specific electrophiles, Current Opinion in Chemical Biology 2015, 24, 18-26, which is incorporated herein by reference in its entirety.

Suitable examples of groups reactive with OH of a serine include the following groups:

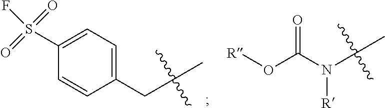

(R′ is H or C₁₋₃ alkyl, R″ is C₁₋₃ alkyl).

Suitable examples of groups reactive with SH of a cysteine include the following groups:

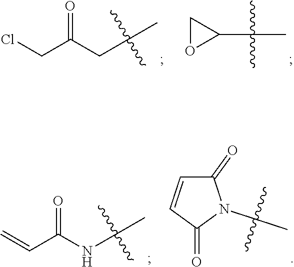

Suitable example of groups reactive with NH₂ of a lysine includes an activated ester of formula:

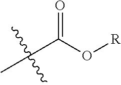

(R is, e.g., N-succinimidyl, N-benzotriazolyl, 4-nitrophenyl, or pentafluorophenyl).

Suitable examples of fluorophores include any fluorescent chemical compound that can re-emit light upon light excitation. The fluorophores can by excited by a light of a wavelength form about 300 nm to about 800 nm, and then emit light of a wavelength from about 350 nm to about 770 nm (e.g., violet, blue, cyan, green, yellow, orange or red light), which can be detected by fluorescent imaging devices, including the ability to measure the intensity of the fluorescence. Suitable examples of fluorophores include AF488, Hydroxycoumarin blue, methoxycoumarin blue, Alexa fluor blue, aminocoumarin blue, Cy2 green (dark), FAM green (dark), Alexa fluor 488 green (light), Fluorescein FITC green (light), Alexa fluor 430 green (light), Alexa fluor 532 green (light), HEX green (light), Cy3 yellow, TRITC yellow, Alexa fluor 546 yellow, Alexa fluor 555 3 yellow, R-phycoerythrin (PE) 480; yellow, Rhodamine Red-X orange, Tamara red, Cy3.5 581 red, Rox red, Alexa fluor 568 red, Red 613 red, Texas Red red, Alexa fluor 594 red, Alexa fluor 633 red, Allophycocyanin red, Alexa fluor 633 red, Cy5 red, Alexa fluor 660 red, Cy5.5 red, TruRed red, Alexa fluor 680 red, and Cy7 red. Absorbance and emission wavelengths of these fluorophores are well known in the art.

In some embodiments, a salt (e.g., pharmaceutically acceptable salt) of a any compound disclosed herein, including any compound of Formula (I), is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, O-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the present disclosure also provides a linker of Formula:

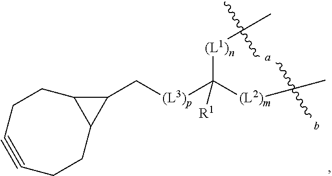

wherein a designates a point of attachment of the linker to a fluorophore, b designates a point of attachment to a protein (e.g., antibody), and L, n, L², m, L³, p, and R¹ are as described herein for Formula (I).

In some embodiments, the present disclosure also provides a linker of Formula:

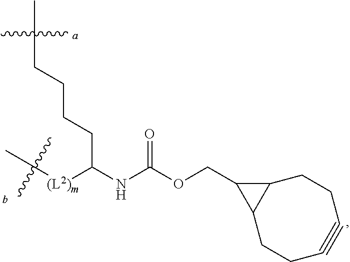

wherein a designates a point of attachment of the linker to a fluorophore, b designates a point of attachment to a protein (e.g., antibody), and L¹, n, L², m, L³, p, and R¹ are as described herein for Formula (I).

### Protein Conjugates

In some embodiments, the tridentate reagents of Formula (I) can be reacted with a protein to obtain a protein conjugate of Formula (II):

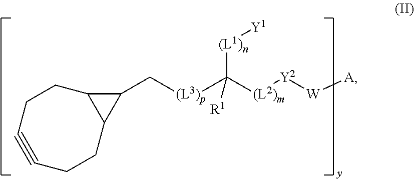

or a pharmaceutically acceptable salt thereof, wherein:
A is a protein;
y is an integer from 1 to 10;
R¹ is selected from H, C₁₋₃ alkyl, and C₁₋₃ haloalkyl;
each L¹ is independently selected from N(R^{N}), O, C(═O), S(═O)₂, C₁₋₆ alkylene, —(OCH₂CH₂)ₓ—, and —(CH₂CH₂O)ₓ—;
n is an integer from 1 to 10;
each L² is independently selected from N(R^{N}), O, C(═O), S(═O)₂, C₁₋₆ alkylene, —(OCH₂CH₂)ₓ—, and —(CH₂CH₂O)ₓ—;
m is an integer from 1 to 10;
each L³ is independently selected from N(R^{N}), O, C(═O), S(═O)₂, C₁₋₆ alkylene, —(OCH₂CH₂)ₓ—, and —(CH₂CH₂O)ₓ—;
wherein each of said C₁₋₆ alkylene groups within L¹, L², or L³ is optionally substituted with (L⁴)ₒ-Y³;
each R^{N} is independently selected from H, C₁₋₃ alkyl, C₁₋₃ haloalkyl, and (L⁴)ₒ-Y³;
each L⁴ is independently selected from N(R^{N1}), O, C(═O), S(═O)₂, C₁₋₆ alkylene, —(OCH₂CH₂)ₓ—, and —(CH₂CH₂O)ₓ—;
each R^{N1} is independently selected from H, C₁₋₃ alkyl, and C₁₋₃ haloalkyl;
p is an integer from 1 to 20;
o is an integer from 1 to 10;
each x is independently an integer from 1 to 2,000;
each Y³ is independently selected from a moiety of formula (i) and a moiety of formula (ii):

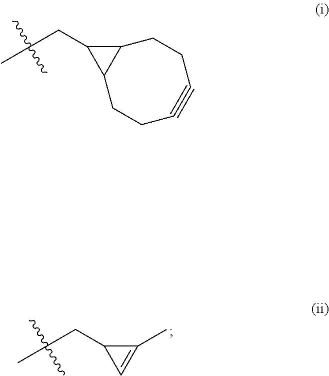

Y¹ is selected from NR^{c1}R^{1A}, OR², and C(═O)R³;
R^{1A}, R², and R³ are each independently selected from a fluorophore;
R^{c1} is selected from H, C₁₋₃ alkyl, and C₁₋₃ haloalkyl;
each W is selected from:
(i) O of a side chain of serine, threonine, or tyrosine of the protein A;
(ii) S of a side chain of cysteine of the protein A;
(iii) NH of a side chain of lysine of the protein A; and
(iv) C(═O) of a side chain of aspartic acid or glutamic acid of the protein A; and
Y² is a residue of a group which, prior to conjugation with the protein A, was a group reactive with a side chain of an amino acid of the protein A.

In some embodiments, y is an integer from 1 to 7. In some embodiments, y is an integer from 1 to 5. In some embodiments, y is selected from 1, 2, 3, 4, 5, 6, or 7.

In some embodiments, y is 1.

In some embodiments, R¹ is H. In some embodiments, R¹ is C₁₋₆ alkyl.

In some embodiments, n is an integer from 1 to 7. In some embodiments, n is an integer from 1 to 5. In some embodiments, n is 1. In some embodiments, n is 2. In some embodiments, n is 3.

In some embodiments, each L¹ is independently selected from NH, O, C(═O), C₁₋₆ alkylene, —(OCH₂CH₂)ₓ—, and —(CH₂CH₂O)ₓ—. In some embodiments, n is an integer from 1 to 5, and each L¹ is selected from NH, O, C(═O), and C₁₋₆ alkylene. In some embodiments, n is 1 and L¹ is C₁₋₆ alkylene. In some embodiments, at least one L¹ is N(R^{N}), and R^{N} is (L⁴)ₒ-Y³. In some embodiments, at least one L¹ is C₁₋₆ alkylene substituted with (L⁴)ₒ-Y³.

In some embodiments, m is an integer from 1 to 7. In some embodiments, m is an integer from 1 to 5. In some embodiments, m is at least 1. In some embodiments, m is an integer from 2 to 10. In some embodiments, m is an integer from 3 to 7.

In some embodiments, m is an integer from 1 to 5, and each L² is independently selected from NH, O, C(═O), C₁₋₆ alkylene, —(OCH₂CH₂)ₓ—, and —(CH₂CH₂O)ₓ—. In some embodiments, m is 4, and each L² is independently selected from NH, C(═O), C₁₋₆ alkylene, and —(OCH₂CH₂)ₓ—. In some embodiments, at least one L² is N(R^{N}), and R^{N} is (L⁴)₀-Y³. In some embodiments, at least one L² is C₁₋₆ alkylene substituted with (L⁴)₀-Y³. In some embodiments, m and L² are selected such that the (L²)ₘ is sufficiently long for the BCN moiety within the Formula (I) to not interfere with a function of a protein (e.g., an antibody) which may be attached to Y² as described further herein.

In some embodiments, p is an integer from 1 to 7. In some embodiments, p is an integer from 1 to 5. In some embodiments, p is at least 1. In some embodiments, p is an integer from 2 to 10. In some embodiments, p is an integer from 3 to 7. In some embodiments, p is an integer from 1 to 15. In some embodiments, p is an integer from 1 to 10. In some embodiments, p is an integer from 1 to 7.

In some embodiments, each L³ is independently selected from NH, O, C(═O), C₁₋₆ alkylene, —(OCH₂CH₂)ₓ—, and —(CH₂CH₂O)ₓ—. In some embodiments, p is an integer from 1 to 5, and each L³ is selected from NH, O, C(═O), and C₁₋₆ alkylene. In some embodiments, p is 3 and each L³ is selected from NH, O, and C(═O). In some embodiments, at least one L³ is C₁₋₆ alkylene substituted with (L⁴)₀-Y³. In some embodiments, at least one L³ is N(R^{N}), and R^{N} is (L⁴)₀-Y³.

In some embodiments, o is an integer from 1 to 7. In some embodiments, o is an integer from 1 to 4. In some embodiments, o is an integer from 1 to 3. In some embodiments, o is an integer from 1 to 5. In some embodiments, each L⁴ is independently selected from NH, O, C(═O), and C₁₋₆ alkylene. In some embodiments, each L⁴ is independently selected from NH, O, and C(═O).

In some embodiments, R^{N} is H. In some embodiments, R^{N} is C₁₋₃ alkyl. In some embodiments, R^{N} is (L⁴)₀-Y³. In some embodiments, R^{N1} is H. In some embodiments, R^{N1} IS C₁₋₃ alkyl.

In some embodiments, x is an integer from 2 to 10. In some embodiments, x is 3, 4, 5, or 6.

In some embodiments of Formula (I):
R¹ is H;
n is an integer from 1 to 5, and each L¹ is selected from NH, O, C(═O), and C₁₋₆ alkylene;
m is an integer from 1 to 5, and each L² is independently selected from NH, O, C(═O), C₁₋₆ alkylene, —(OCH₂CH₂)ₓ—, and —(CH₂CH₂O)ₓ—;
x is an integer from 2 to 10;
p is an integer from 1 to 5;
each L³ is independently selected from N(R^{N}), O, C(═O), and C₁₋₆ alkylene, wherein said C₁₋₆ alkylene is optionally substituted with (L⁴)ₒ-Y³;
each R^{N} is independently selected from H and (L⁴)ₒ-Y³;
o is an integer from 1 to 5; and
each L⁴ is independently selected from NH, O, C(═O), and C₁₋₆ alkylene.

In some embodiments, the compound of Formula (I) comprises one Y³ group.

In some embodiments, p is an integer from 1 to 3, and each L³ is independently selected from NH, O, and C(═O).

In some embodiments, the conjugate of Formula (II) has formula:

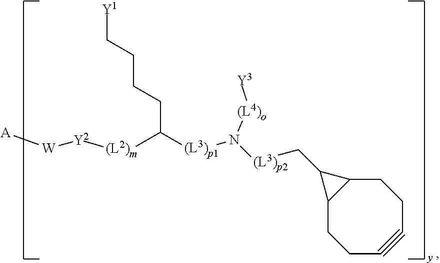

or a pharmaceutically acceptable salt thereof, wherein the sum of p1 and p2 is less than p by at least 1.

In some embodiments, the conjugate of Formula (II) has formula:

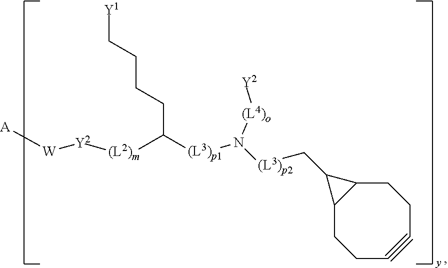

or a pharmaceutically acceptable salt thereof, wherein the sum of p1 and p2 is less than p by at least 1.

In some embodiments, the Y³ (if present) is a moiety of formula (i):

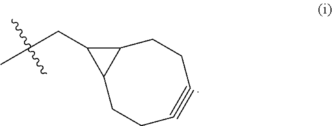 [SMILES: *CC1C2CCC#CCCC12]

In some embodiments, the Y³ (if present) is a moiety of formula (ii):

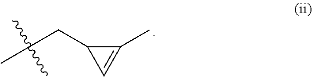 [SMILES: *CC1C=C1C]

In some embodiments of Formula (II):
R¹ is H;
n is 1 and L¹ is C₁₋₆ alkylene;
m is 4, and each L² is independently selected from NH, C(═O), C₁₋₆ alkylene, and —(OCH₂CH₂)ₓ—;
p is 3, and each L³ is independently selected from NH, O, and C(═O); and
x is an integer from 2 to 10.

In some embodiments, the conjugate of Formula (II) has formula:

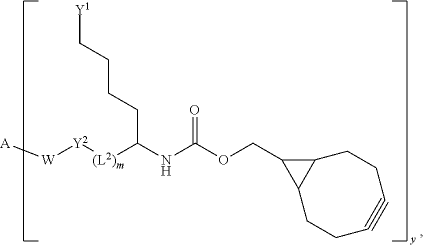

or a pharmaceutically acceptable salt thereof.

In some embodiments, the conjugate of Formula (II) has formula:

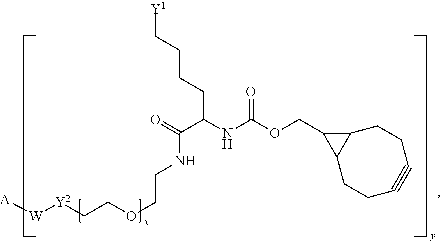

or a pharmaceutically acceptable salt thereof.

In some embodiments, (L¹)ₙ comprises a side chain of an amino acid (e.g., lysine, serine, threonine, cysteine, tyrosine, aspartic acid, or glutamic acid), and Y¹ comprises the terminal functional group of the side chain of the amino acid (e.g., N, O, S, or C(═O)).

In some embodiments, y is an integer from 4 to 6. In some embodiments, y is an integer from 1 to 10. In some embodiments, y is 1. In some embodiments, y is 4. In some embodiments, y is 5. In some embodiments, y is 6. In some embodiments, y is 7.

In some embodiments, Y¹ is NHR^{1A}.

In some embodiments, Y¹ is OR².

In some embodiments, Y¹ is C(═O)R³.

The fluorophore in any one of the R^{1A}, R², and R³ can be any one of the fluorophores described herein for Formula (I). In some embodiments, the fluorophore of Formula (II) is selected from AF488, AF647, AF594, and AF555.

In some embodiments, Y², prior to conjugation to protein A, is any one of the reactive Y² groups described herein for Formula (I). Suitable examples of Y² groups of Formula (II) include C(═O), and any one of the following moieties:

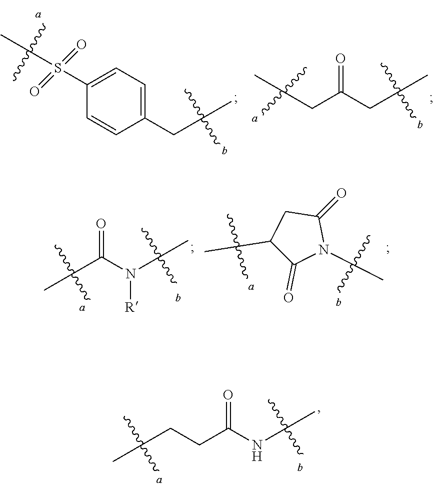

wherein a is point of attachment of the moiety to W, and b is a point of attachment of the moiety to L².

In some embodiments, W is O of a side chain of serine, threonine, or tyrosine of the protein A. In some embodiments, W is S of a side chain of cysteine of the protein A. In some embodiments, W is NH of a side chain of lysine of the protein A. In some embodiments, W is C(═O) of a side chain of aspartic acid or glutamic acid of the protein A.

In some embodiments, each Y² is C(═O) and each W is NH of a side chain of lysine of the protein A. In some embodiments, each Y² is C(═O) and at least one W is S of a side chain of cysteine of the protein A.

In some embodiments, R^{c1} is H. In some embodiments, R^{c1} is C₁₋₃ alkyl.

In some embodiments, the protein is selected from an antibody, an antibody fragment, an engineered antibody, a peptide, and an aptamer. In some embodiments, the protein is an antibody. In some embodiments, the antibody is specific to an antigen which is a biomarker of a disease or condition. In some embodiments, the disease or condition is cancer. In some embodiments, the disease or conditions is a disease of the immune system. Suitable examples of such diseases include severe combined immunodeficiency (SCID), autoimmune disorder, familial Mediterranean fever and Crohn's disease (inflammatory bowel disease), arthritis (including rheumatoid arthritis), Hashimoto's thyroiditis, diabetes mellitus type 1, systemic lupus erythematosus, and myasthenia gravis. In some embodiments, the antigen is a biomarker of immune system response to a viral infection or a vaccine. Suitable example of viral infections include infections caused by a DNA virus, an RNA virus, or a coronavirus. One example of a viral infection is influenza. Another example of a viral infection is a coronavirus infection, such as COVID-19 (caused by SARS-CoV-2), Middle East respiratory syndrome (MERS) (caused by MERS-CoV), or severe acute respiratory syndrome (SARS) (caused by SARS-CoV). In some embodiments, the antigen is a biomarker of a cytokine storm. A cytokine storm can occur as a result of an infection (e.g., a viral infection as described herein), a vaccine (e.g., a vaccine against any of the viral infections described herein), an autoimmune condition, or other disease. Suitable examples of such cytokines include pro-inflammatory cytokines such as IL-6, IL-1, TNF-α, or interferon. In some embodiments, the antibody is specific to an antigen indicative of an immune system response to COVID-19 (including cytokine storm).

Suitable examples of biomarkers include CD45, CD3, CD4, CD8, PD-1, PD-L1, CD11b, F4/80, CD163, CD206, Ly6G, CD11c, and MHCII. Any other biomarker the presence of which in the cell (e.g., on the cell surface) is known in the art to be indicative of severity of the disease, or to be indicative of the presence of some disease state, can be used as an antigen for the antibody A of the Formula (B) or Formula (II). Some examples of cancer biomarkers include alpha fetoprotein (AFP), CA15-3, CA27-29, CA19-9, CA-125, calcitonin, calretinin, carcinoembryonic antigen, CD34, CD99MIC 2, CD117, chromogranin, chromosomes 3, 7, 17, and 9p21, cytokeratin (various types: TPA, TPS, Cyfra21-1), desmin, epithelial membrane antigen (EMA), factor VIII, CD31 FL1, glial fibrillary acidic protein (GFAP), gross cystic disease fluid protein (GCDFP-15), HMB-45, human chorionic gonadotropin (hCG), immunoglobulin, inhibin, keratin (various types), lymphocyte marker (various types, MART-1 (Melan-A), myo D1, muscle-specific actin (MSA), neurofilament, neuron-specific enolase (NSE), placental alkaline phosphatase (PLAP), prostate-specific antigen (PSA), PTPRC (CD45), S100 protein, smooth muscle actin (SMA), synaptophysin, thymidine kinase, thyroglobulin (Tg), thyroid transcription factor-1 (TTF-1), tumor M2-PK, and vimentin.

In some embodiments, the biomarker is selected from CD45, CD3, CD8, CD4, FoxP3, NK1.1, CD19, CD20, CD11b, F4/80, CD11c, Ly6G, Ly6C, MHCII, PD-1, PD-L1, granzyme B, IFNγ, CK5/6, p16, CD56, CD68, CD14, CD1a, CD66b, CD39, TCF1, IL-120, and CD163. In some embodiments, the antibody is specific to PD-1 (e.g., pembrolizumab, nivolumab, or cemiplimab). In some embodiments, the antibody is specific to PD-L1 (e.g., atezolizumab, avelumab, or durvalumab).

In some embodiments, the present disclosure provides a composition comprising a protein conjugate of Formula (II), or a pharmaceutically acceptable salt thereof, and an inert carrier. In some embodiments, the composition is an aqueous solution (i.e., the inert carrier is water). The aqueous solution may be a buffer, such as any buffer containing inert carrier such as water, phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, or any combination thereof. Some examples of buffers include Dulbecco's phosphate-buffered saline (DPBS), phosphate buffered saline, and Krebs-Henseleit Buffer. The pH of the buffer may be from about 5 to about 9, for example pH may be 6-8. The compound of Formula (I), or a salt thereof, wherein Y² is a group reactive with a protein, may be admixed with the protein (e.g., antibody) in any of the aqueous solutions described here to obtain the compound of Formula (II).

A composition (e.g., an aqueous solution) comprising the compound Formula (II), may be used to treat a cell (e.g., a cell containing a biomarker) to image the cell using the fluorophore of the Formula (II).

### Methods of Cellular Analysis

Accordingly, the present disclosure provides a method of examining a cell or a component of a cell (e.g., nucleus of a cell), the method comprising:
(i) contacting the cell with a conjugate of Formula (II) comprising the fluorophore, or a pharmaceutically acceptable salt thereof, or a pharmaceutical composition comprising same;
(ii) imaging the cell with an imaging technique; and
(iii) after (ii), contacting the cell with a compound of Formula (III):

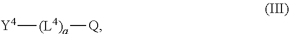

or a pharmaceutically acceptable salt thereof, wherein:
Y⁴ is selected from N₃ and a moiety of formula (iii):

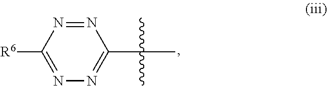

R⁶ is selected from H, C₁₋₆ alkyl, and C₁₋₆ haloalkyl, wherein said C₁₋₆ alkyl is optionally substituted with OH, NH₂, or COOH;
each L⁴ is independently selected from N(R^{N}), O, C(═O), S(═O)₂, C₁₋₆ alkylene, C₆₋₁₀ arylene, C₆₋₁₀ perfluoroarylene, —(OCH₂CH₂)ₓ—, and —(CH₂CH₂O)ₓ—;
a is an integer from 1 to 10;
each R^{N} is selected from H and C₁₋₃ alkyl;
x is an integer from 1 to 2,000; and
Q is a quencher,
wherein the contacting of step (iii) results in decrease of the fluorescence of the fluorophore in the conjugate of Formula (II), or a pharmaceutically acceptable salt thereof.

Without being bound by a theory, it is believed that when the cell is contacted with the protein conjugate of Formula (II) in step (i), the protein A (e.g., antibody) binds to its antigen on the surface of the cell or in the cytoplasm of the cell (or in a nucleus of the cell), and, therefore, the cell or its component can be imaged by detecting fluorescence of the fluorophore in the Formula (II).

In some embodiments, the imaging technique of step (ii) is a fluorescence imaging, such as microscopy, imaging probes, and spectroscopy. The fluorescence imaging devices include an excitation source, the emitted light collection source, optionally optical filters, and a means for visualization (e.g., a digital camera for taking fluorescence imaging photographs). Suitable examples of fluorescence imaging include internal reflection fluorescence microscopy, light sheet fluorescence microscopy, and fluorescence-lifetime imaging microscopy. Suitable imaging techniques are described, for example, in Rao, J. et al., Fluorescence imaging in vivo: recent advances, Current Opinion in Biotechnology, 18, (1), 2007, 17-25, which is incorporated herein by reference in its entirety.

In some embodiments, Y⁴ is N₃.

In some embodiments, the compound of Formula (III) has formula:

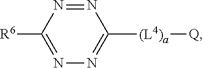

or a pharmaceutically acceptable salt thereof.

In some embodiments, R⁶ is H. In some embodiments, R⁶ is CH₃. In some embodiments, R⁶ is C₁₋₆ alkyl, optionally substituted with OH, NH₂, or COOH.

In some embodiments, a is an integer from 4 to 10. In some embodiments, a is an integer from 3 to 7. In some embodiments, a is at least 3. In some embodiments, a is 1, 2, 3, 4, 5, 6, or 7. In some embodiments, a is an integer from 1 to 7, and each L⁴ is independently selected from NH, C(═O), C₁₋₆ alkylene, C₆₋₁₀ arylene, C₆₋₁₀ perfluoroarylene, and —(CH₂CH₂O)ₓ—. In some embodiments, each x is an integer from 1 to 10. In some embodiments, x is 1, 2, 3, 4, or 5.

In some embodiments, the quencher Q is a fluorescence quencher. Suitable examples of fluorescence quenchers include aromatic azo compounds and phenazine derivatives. In some examples, the fluorescence quencher is BHQ0, BHQ1, BHQ2, BHQ3, BHQ10, or IRDye QC-1. In some embodiments, the quencher is selected from dabcyl, IowaBlack quenchers, ATTO 540Q, ATTO 575Q, ATTO 580Q, ATTO 612Q, BBQ-650, QXL quenchers, and TIDE quenchers.

In some embodiments, the compound of Formula (III) is selected from any one of the following compounds:

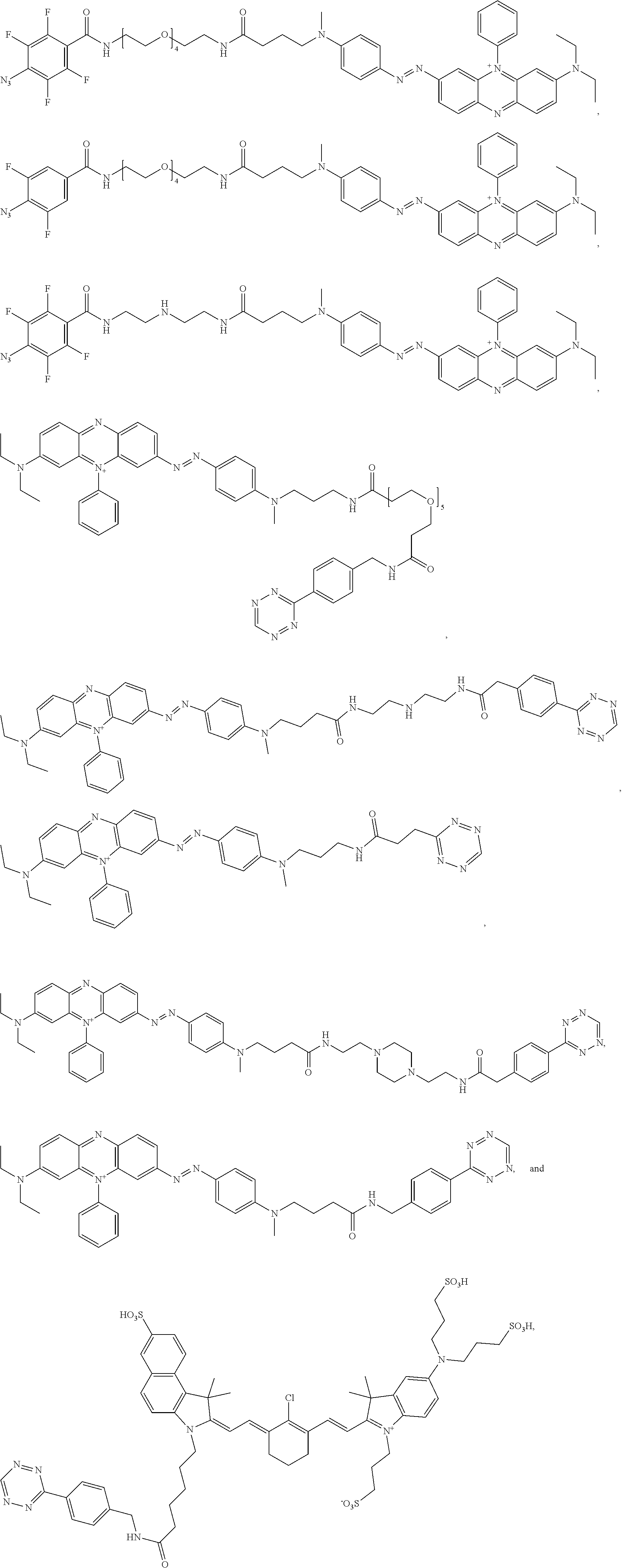

or a pharmaceutically acceptable salt thereof.

In some embodiments, the contacting of step (iii) results in decrease of the fluorescence (or complete quenching of the fluorescence) of the fluorophore in the conjugate of Formula (II). Without being bound by a theory, it is believed that the quencher Q can quench the fluorescence of the fluorophore of Formula (II) through contact (static) quenching. Without being bound by a theory, it is believed that the quencher Q can also quench the fluorescence of the fluorophore of Formula (II) through FRET quenching, that is, the excited fluorophore instead of emitting light transfers energy to the quencher through space. In the absence of the quencher, the fluorophore would have emitted the light, which could have been detected. In some embodiments, the Q of Formula (III) and the fluorophore of Formula (II) are selected such that the emission spectrum of the fluorophore substantially overlaps with the absorption spectrum of the quencher Q.

Without being bound by a theory, in one example, it is believed that the BCN moiety in the protein conjugate of Formula (II) reacts with the tetrazine moiety of the Formula (III) to produce a protein conjugate of Formula (IV), as shown, for example, in Scheme 1.

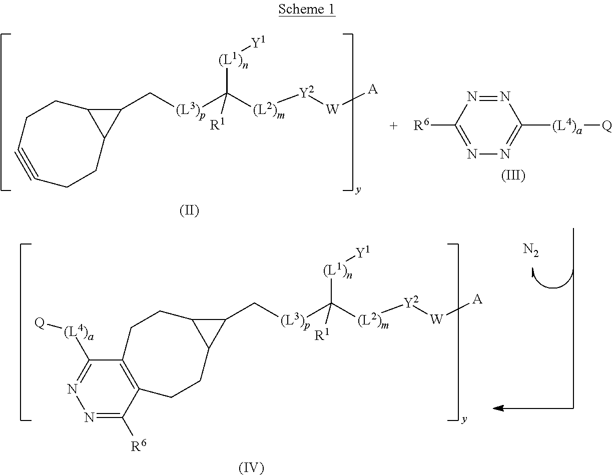

Referring to Scheme 1, the BCN fragment of Formula (II) engages in inverse-demand Diels Alder with the tetrazine of Formula (III) followed by a retro-Diels Alder reaction to eliminate nitrogen gas. Through this ligation, the fluorophore of Y¹ and the quencher Q in the compound of Formula (IV) are covalently connected and well as positioned in close special proximity. Without being bound by a theory, it is believed that the spatial proximity between Q and the fluorophore of Y¹, created by covalent link between these groups, allows for efficient quenching of fluorescence.

In some embodiments, the present disclosure provides a tridentate linker of formula:

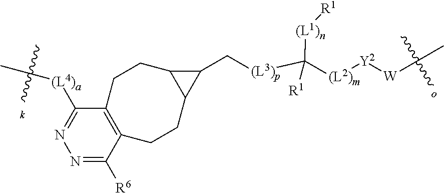

wherein 1 denotes a point of attachment to a fluorophore, o denotes a point to attachment to a protein, k denotes a point of attachment to fluorescence quencher, L¹, n, L², m, R¹, L³, p, Y², and W are as described herein for Formula (II), and L⁴, a, and R⁶ are as described herein for Formula (III).

### Methods of Use

In some embodiments, the present disclosure provides a method of profiling a cell, the method comprising (i) obtaining the cell from a subject, and (ii) examining the cell according to the methods of cellular analysis described herein.

In some embodiments, the present disclosure provides a method of examining a cell using a cytometry technique, the method comprising (i) obtaining the cell from a subject, and (ii) examining the cell according to the method of cellular analysis described herein. Suitable examples of cytometry techniques include image cytometry, holographic cytometry, Fourier ptychography cytometry, and fluorescence cytometry.

In some embodiments, the present disclosure provides a method of diagnosing a disease or condition of a subject by examining pathology of a cell obtained from the subject, the method comprising (i) obtaining the cell from a subject, and (ii) examining the cell according to the method of cellular analysis described herein.

In some embodiments, the present disclosure provides a method of monitoring progression of disease or condition (or monitoring efficacy of treatment of disease or condition) of a subject by examining pathology of a cell obtained from the subject, the method comprising (i) obtaining the cell from the subject, and (ii) examining the cell according to the method of cellular analysis described herein. The method allows to guide therapeutic regimens based on the results of examination of the cell according to the methods, and to provide individualized treatments.

In some embodiments, the present disclosure provides a method of monitoring efficacy of treatment of cancer. Suitable examples of cancer treatments include chemotherapy, radiation therapy, and surgery, or any combination of the foregoing. Suitable examples of chemotherapeutic treatments include abarelix, aldesleukin, alitretinoin, allopurinol, altretamine, anastrozole, arsenic trioxide, asparaginase, azacitidine, bexarotene, bleomycin, bortezomib, busulfan, calusterone, capecitabine, carboplatin, carmustine, chlorambucil, cisplatin, cladribine, clofarabine, cyclophosphamide, cytarabine, dacarbazine, dactinomycin, dalteparin, dasatinib, daunorubicin, decitabine, denileukin, dexrazoxane, docetaxel, doxorubicin, dromostanolone propionate, emtansine, epirubicin, eribulin, erlotinib, estramustine, etoposide, everolimus, exemestane, fentanyl citrate, filgrastim, floxuridine, fludarabine, fluorouracil, fruquintinib, fulvestrant, gefitinib, gemcitabine, ozogamicin, goserelin acetate, histrelin acetate, tiuxetan, idarubicin, ifosfamide, imatinib, interferon α2a, irinotecan, ixabepilone, lapatinib, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, oxaliplatin, paclitaxel, pamidronate, panitumumab, pegaspargase, pegfilgrastim, pemetrexed, pentostatin, pipobroman, plicamycin, procarbazine, quinacrine, rasburicase, sorafenib, streptozocin, sulfatinib, sunitinib, sunitinib, tamoxifen, temozolomide, teniposide, testolactone, thalidomide, thioguanine, thiotepa, topotecan, toremifene, tretinoin, uracil mustard, valrubicin, vinblastine, vincristine, vinorelbine, volitinib, vorinostat, and zoledronate, or a pharmaceutically acceptable salt thereof.

In some embodiments, cancer treatment comprises administering to a patient an antibody useful in treating cancer. Suitable examples of such antibodies include pembrolizumab, nivolumab, cemiplimab, atezolizumab, avelumab, durvalumab, abagovomab, adecatumumab, afutuzumab, alacizumab pegol, altumomab pentetate, amatuximab, anatumomab mafenatox, apolizumab, arcitumomab, bavituximab, bectumomab, belimumab, bevacizumab, bivatuzumab mertansine, blinatumomab, brentuximab vedotin, cantuzumab mertansine, cantuzumab ravtansine, capromab pendetide, cetuximab, citatuzumab bogatox, cixutumumab, clivatuzumab tetraxetan, dacetuzumab, demcizumab, detumomab, drozitumab, ecromeximab, eculizumab, elotuzumab, ensituximab, epratuzumab, etaracizumab, farletuzumab, figitumumab, flanvotumab, galiximab, gemtuzumab ozogamicin, girentuximab, ibritumomab tiuxetan, imgatuzumab, ipilimumab, labetuzumab, lexatumumab, lorvotuzumab mertansine, nimotuzumab, ofatumumab, oregovomab, panitumumab, pemtumomab, pertuzumab, tacatuzumab tetraxetan, tositumomab, trastuzumab, totumumab, rituximab, alemtuzumab, durvalumab, ofatumumab, elotuzumab, and zalutumumab.

Suitable examples of cancer treatments also include immunotherapy. In some embodiments, the cancer treatment comprises a checkpoint inhibitor. In some embodiments, the checkpoint inhibitor is selected from anti-PD-1, anti-PD-L1, anti-CTLA-4, anti-CD20, anti-SLAMF7, and anti-CD52 (e.g., any one of the anticancer antibodies described above).

In some embodiments, the present disclosure provides a method of detecting a disease biomarker in a cell, the method comprising (i) obtaining the cell from a subject, and (ii) examining the cell according to the method of cellular analysis described herein.

In some embodiments, the cell is obtained from the subject using image-guided biopsy, fine needle aspiration (FNA), surgical tissue harvesting, punch biopsy, liquid biopsy, brushing, swab, touch-prep, fluid aspiration or blood analysis. In some embodiments, the cell is obtained from the subject using fine needle aspiration (FNA). In some embodiments, the cell is obtained from a tissue sample, such as a paraffin embedded (FFPE) tissue sample, a fresh tissue sample, or a frozen tissue sample. In some embodiments, the cell is selected from a cancer cell, an immune system cell, and a host cell (the methods of the present disclosure are useful for hepatocyte profiling in liver disease etc.). In some embodiments, the cell is a cancer cell. In some embodiments, the cancer cell is infected with human papillomavirus (HPV). In some embodiments, the cancer is caused by human papillomavirus (HPV). In some embodiments, a cellular sample obtained from the subject or from a tissue of the subject is scant or abundant. In some embodiments, the methods and reagents of the present disclosure are suitable for cellular samples and tissue samples containing any quantity of cells.

In some embodiments, the disease or condition (which can be diagnosed, monitored, or biomarker of which can be detected using the present methods) is cancer. In some embodiments, the methods disclosed herein allow to determine the composition of the tumor microenvironment. Suitable examples of cancer include lymphoma, breast cancer, skin cancer, head and neck cancer, head and neck squamous cell carcinoma (HNSCC), and oral cancer. Other examples of cancers include colorectal cancer, gastric (gastrointestinal) cancer, leukemia, melanoma, and pancreatic cancer, hepatocellular carcinoma, ovarian cancer, endometrial cancer, fallopian tube cancer, lung cancer, medullary thyroid carcinoma, mesothelioma, sex cord-gonadal stromal tumor, adrenocortical carcinoma, synovial sarcoma, bladder cancer, smooth muscle sarcoma, skeletal muscle sarcoma, endometrial stromal sarcoma, glioma (astrocytoma, ependymoma), rhabdomyosarcoma, small, round, blue cell tumor, neuroendocrine tumor, small-cell carcinoma of the lung, thyroid cancer, esophageal cancer, and stomach cancer. The technology is useful for any cancer detectable and compatible with biopsy by direct visualization, palpation, or image guidance.

In some embodiments, the cell is an immune cell. In some embodiments, the cell is selected from a hematopoietic cell, a T cell, a B cell, a NK cell, a myeloid cell, a macrophage, a dendritic cell, a neutrophil, and a monocyte.

Application of these methods is described more fully as follows.

The linkers, reagents, compounds, and methods of the present disclosure can be used at a point-of-care settings. In developed countries, repeat biopsies of ever-smaller lesions are straining accuracy and throughput, while low- and middle-income countries face extremely limited pathology and imaging resources, large case loads, convoluted and inefficient workflows, and lack of specialists. Advantageously, the compounds and methods described here allow for highly precise analysis of scant cancer samples, particularly those obtained by fine needle aspiration of mass lesions.

Accordingly, in some embodiments, the present disclosure provides an image cytometer that allows for automated cell phenotyping of scant cell samples. Various device applications for the methods and compounds of the present applications are described below.

Cellular cancer diagnostics are essential to clinical decision making: establishing the correct diagnosis, choosing the appropriate treatment, enrolling patients in experimental trials, assessing therapeutic efficacy and/or re-staging disease. Today, cancer specimens are commonly obtained by image-guided biopsy, fine needle aspiration (FNA), surgical tissue harvesting, punch biopsies, brushings, swabs, touch-preps, fluid aspiration or blood analysis (leukemia, lymphoma, liquid biopsies). Some of these methods (core and open surgical biopsies for histopathology) yield abundant tissue for sectioning and staining while others (FNA, brushings, touch-preps for cytopathology) yield scant cellular materials. FNA can often be obtained with minimal intervention using small-gauge needles (20-25 G), have very low complication rates and are generally well tolerated.

Rapid onsite assessment of cellular specimens has become increasingly important to narrowing the time between intervention and initiation of therapy, assuring specimen quality for subsequent diagnoses and minimizing sample degradation and loss during transport. The current workflows are still labor intensive and often centralized, requiring extensive sample processing and expert cytopathology review. Digital cytopathology and whole-slide imaging have been implemented but also require significant time, labor and investment. Taken together, these factors limit throughput, cost and global reach. A particular challenge is reliably analyzing scant cells either via manual imaging (requiring a trained cytopathologist reviewing an entire slide) or automated analysis (incorporating machine learning routines for automated diagnoses).

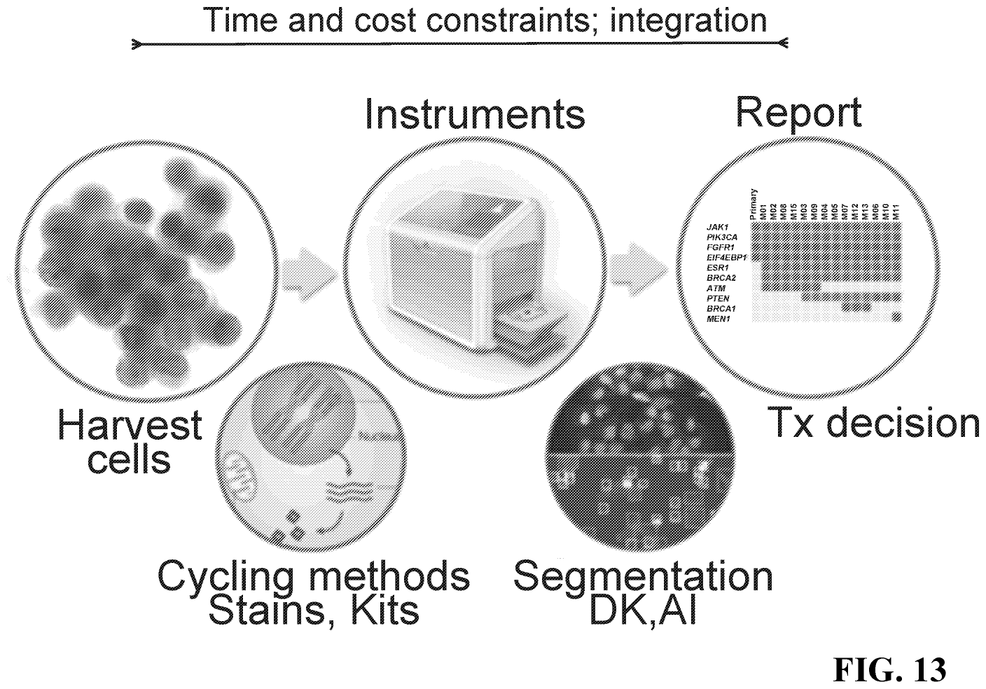

The present compounds and methods can be used in automated molecular image cytometers that use advanced materials, engineering and artificial intelligence (AI) for digital cell phenotyping. These new “all-in-one” systems address a potentially large clinical need by enabling advanced cellular diagnostics well suited to: 1) a global health market that is currently underserved; 2) repeat sampling at ultra-low morbidity since smaller needles are used (important for repeat sampling in clinical trials); 3) faster turn-around times (time saved by point-of-care analysis and neither embedding nor staining cores); 4) better and automated quality control and 5) invoking automation to reduce both time to diagnosis and the variability of interpretation. In addition, the present compounds and methods can be used in low-cost flow cytometers, liquid biopsies focusing on cfDNA, exosomes, circulating tumor cells (CTCs), and genomic screening tools (F1CDx, MSK-IMPACT). In some embodiments, the present compounds and methods are useful in automated analysis of cellular specimens obtained by tumor FNA (FIG. 13). In some embodiments, the present disclosure provides, in addition to the miniaturized and automated cytometry systems for desktop, point-of-care application described here, a high-throughput device useful for analysis of samples in centralized laboratories, such as CLIA labs.

### Generic Cellular Stains

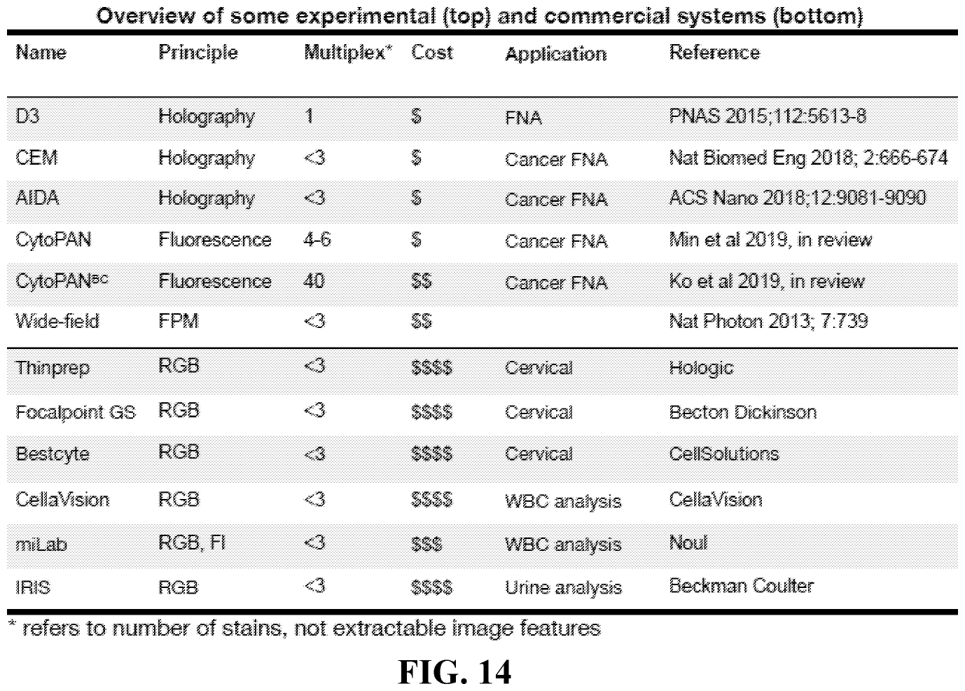

Conventional cytopathology largely relies on chromogenic stains such as hematoxylin and eosin (H&E), Papanicolaou (PAP) and Giemsa. Stained specimens are reviewed by cytopatholgists who evaluate cells for a number of parameters, for example, nuclear/cytoplamic ratio, nuclear features, mitoses, clusters, cell uniformity, and cohesiveness. Such analyses can be automated but are inherently limited, resulting in variable diagnostic accuracies and lack of molecular information. Most commercial cell analyzers (FIG. 14) use this approach for automated white blood cell (WBC) analysis rather than cancer detection. Alternative dyes to investigate nuclear morphology (aneuploidy, segmentation) include DAPI, acridine orange, ethidium iodide, propidium iodide or flavins. Given the limitations of generic chromogenic staining, immunostaining for cancer-associated and host cell markers has emerged as an alternative and is being used widely in CTC analysis.

### Antibody Staining

Antibodies are increasingly used in cytopathology and the standard is to perform one stain at a time, primarily using immunocytology (absorption measurements of antibody-enzyme-mediated chemical reactions) rather than immunofluorescence (emission measurements of fluorescently labeled antibodies). The compounds and methods of the present disclosure allow to detect a key molecular biomarker (e.g., cancer biomarker) while allowing morphological assessment of cells (e.g., cancer cells), for example, HER2 immunostaining in H&E slides.

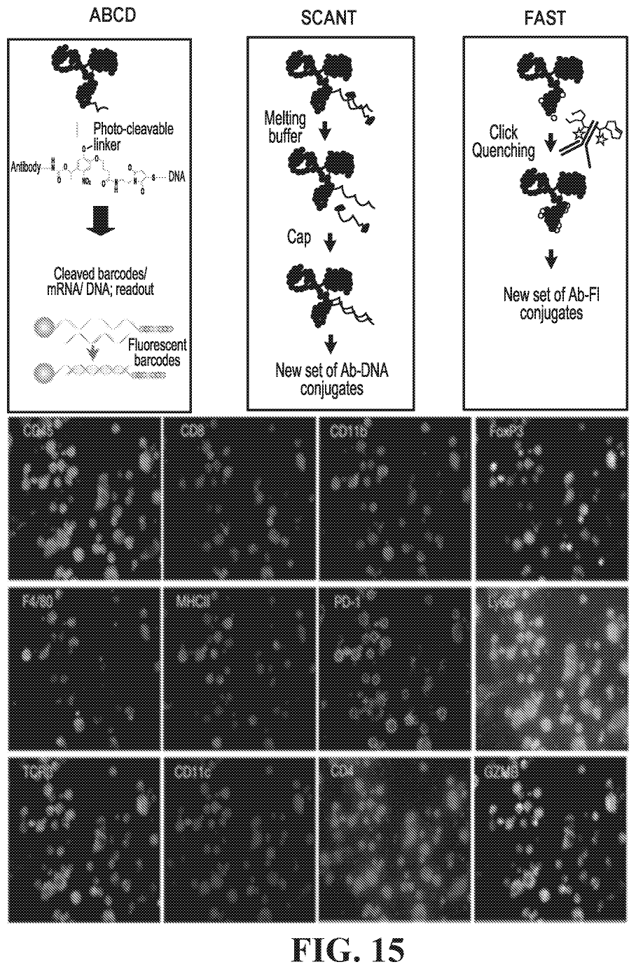

Multichannel fluorescence imaging (typically 4-6 channels) can be used to obtain more stains on a given cell, similar to flow cytometry, albeit at the cost of detailed cellular morphological information. To further improve the number of channels and markers (>20), cycling technologies have been developed that can repeatedly stain, destain and re-stain cancer tissues, ultimately allowing the number of markers per cell to be increased. This in turn facilitates deeper cell-by-cell profiling, pathway analysis and immunoprofiling in scant FNA. Most cycling methods were originally developed for paraffin-embedded tissue sections that can withstand harsh destaining conditions. Unfortunately, these harsh conditions, requiring oxidants for bleaching, are often incompatible with FNA samples. Furthermore, it was not uncommon for early cycling technologies to require days to process samples. Several different cell-compatible cycling technologies have been developed in recent years (FIG. 15). The more recent SCANT (single cell analysis for tumor phenotyping) method (FIG. 15) was shown to be robust and useful for pathway analysis in a clinical setting. One of the obstacles with SCANT, however, was its comparatively low SNR and relatively long destaining times (0.5-1 hour), similar to other cycling techniques. The methods and compounds of the present disclosure (e.g., FAST method) bypasses these shortcomings and allows extremely fast cycling (>95% quenching in <10 sec; FIG. 15).

### Choice of Biomarkers

Selecting appropriate molecular markers is essential to identifying cells (e.g., cancer cells), differentiating them from host cells and profiling a growing number of treatment-relevant immune cells. While host cell markers have been thoroughly characterized by extensive flow cytometry studies, epithelial cancer markers are more diverse and thus require more stains. Furthermore, tumor markers are typically only expressed in a fraction of cancer cells and cases. The compounds and methods of the present disclosure allow to stain the following combinations of biomarkers: i) EpCAM, cytokeratins (CK), CD45 and CD16; ii) multi-marker combinations comprising for example EGFR, EpCAM, MUC1 and WNT2 (“Quad” marker”); iii) HER2, ER/PR for breast cancer; iv) CD19/20, k, 1, Ki67 for lymphoma; v) EGFR, TTF1, chromogranin, synaptophysin for lung cancer; vi) EpCAM, calretinin, CD45, vimentin (ATCdx) for ovarian cancer and markers for mutated proteins such as KRASG12d, EGFRv3, IDH1132G and BRAFV600E, among others.

### Optimizing Materials for Cellular Analysis

Freshly obtained clinical samples have to be fixed, stained and captured on glass before they can be analyzed. All of these steps require careful optimization and often modified materials. Fixation can usually be done in paraformaldehyde, methanol/propanol or other commercially available mixes such as CytoRich Red (CRR). We have found empirically that some samples are better preserved in 50% diluted CRR, while fixation length (ideally 15-30 minutes) is less critical.

Immunostaining is best performed in small plastic vials by adding antibody reagents to cells in a staining buffer. Antibody-fluorochrome stability, quality control issues and limited access to basic tools (centrifuge, filters) are notable hurdles when using immunostains in remote areas and in point-of-care (POC) devices. Use of lyophilized antibodies and “cocktails” that contain all necessary ingredients can reduce variability. An alternative is to stain cells directly on glass slides after capture. Capturing cells on a glass slide is also critical to ensure that cells can be brought to the focal plane. Capture can be done using biological “glues” such as dopamine, biotin/neutravidin or polylysine as slide coatings. Alternatively, glass slides can be coated with capture antibodies. Irrespective of the method used, careful validation is required for different applications. Non-specific binding is typically reduced by coating slides with blocking materials such as BSA or PEG polymers. In order to simplify sample handling and processing, commercial systems may adapt cartridges to perform all of the above steps in a single platform.

### Image Cytometry Systems

To inspect heterogeneous cell populations with statistical confidence, image cytometers must visualize large numbers of individual cells. Conventional geometric optics, however, are inherently constrained by the so-called space-bandwidth product (SBP and therefore produce megapixel information. This translates to a familiar experience: common microscopes have either wide field-of-view (FOV) at low resolution or small FOV at high spatial resolution but not both at the same time.

Most laboratory imaging systems overcome this limit by combining high-magnification optics with scanning stages to automatically scan slides and then transmit the information. The technologies of whole side imaging (WSI) and digital cytopathology have progressed over the years but challenges remain. Two key issues in digital cytopathology are i) focusing and ii) the remaining need for expert review. The focusing issue has largely been solved via either autofocusing hardware/software or 3D imaging of thick z-stacks. Autofocusing software often uses either a least squared or a mean value method to locate the ideal focus plane. 3D imaging, such as microscopy with optical sectioning, requires confocal laser scanning microscopy (CLSM), two-photon (2P) microscopy, structured illumination microscopy (SIM), light sheet fluorescence microscopy (LSFM) or Inverted selective plane illumination microscopy (iSPIM). All of these methods entail expensive instrumentation, require expert users and often generate/produce very large data sets. As such, this particular approach limits deployment in resource-constrained remote locations.

New technological advances increasingly enable automated molecular image cytometry, which is particularly helpful for POC use. Computational optics, wherein optically encoded images are digitally interpreted, can expand the SBP beyond optics' physical limit. Advances in optoelectronics and micro-optics further enable the construction of compact, easy-to-control, yet high-performance systems. Using these approaches can also decrease the overall system cost, as optoelectronical parts and computation have become inexpensive. Here we highlight three emerging modalities embodying these new concepts: digital holography, Fourier ptychography and miniaturized fluorescence cytometry.

### Miniaturized Fluorescence Cytometry

As the list of known tumor markers grows, the need for multiplexed cellular profiling also increases, largely driven by interest in improving diagnostic accuracy, allowing patient triaging and facilitating molecularly based treatment decisions. Conventional immunocytology, which is based on chromogenic staining and brightfield microscopy, typically probes only for a few markers simultaneously. Fluorescent imaging, particularly in combination with cycling technologies, is a potent approach to in-depth multiplexing; a major technical challenge is to transform bulky, expensive microscopes into compact, affordable equivalents for POC uses. Fortunately, recent advances in optoelectronics have made available high-quality mini optical 8 parts, prompting new systems engineering. For example, small LEDs can deliver sufficient power to replace conventional lamps or lasers as an excitation light source, and the photosensitivity of semiconductor imagers has improved significantly for reliable low-light detection. Another opportunity is to augment manual image curation with automated analyses using machine learning approaches.

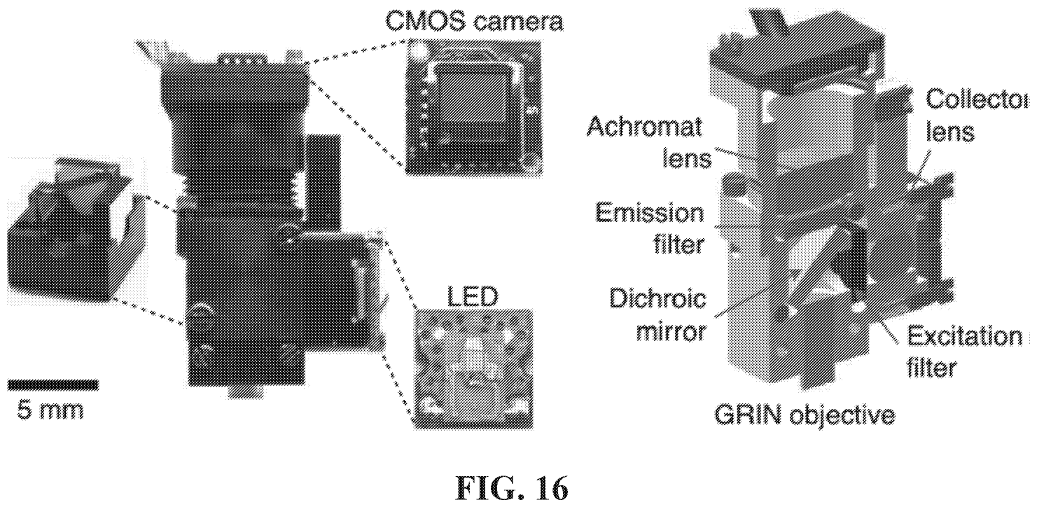

Thumb-sized fluorescent microscopes (“miniscopes”) integrate optical components into a single device (FIG. 16). Using a gradient refractive index (GRIN) objective lens makes possible to shorten the optical path and drastically reduce system size (2.4 cm³, 1.9 g). Such a small form factor allowed the scope mountable on an animal's head with minimal interruption to its natural behavior and to image live neuron cells. As potential POC applications, miniscopes have been used for cell profiling and bacterial detection. In addition, a miniscope array performed large-area imaging without scanning, taking advantage of the scope's small lateral size (˜5 mm). System modification and computational processing enabled two-photon excitation, volumetric rendering or lens-less imaging.

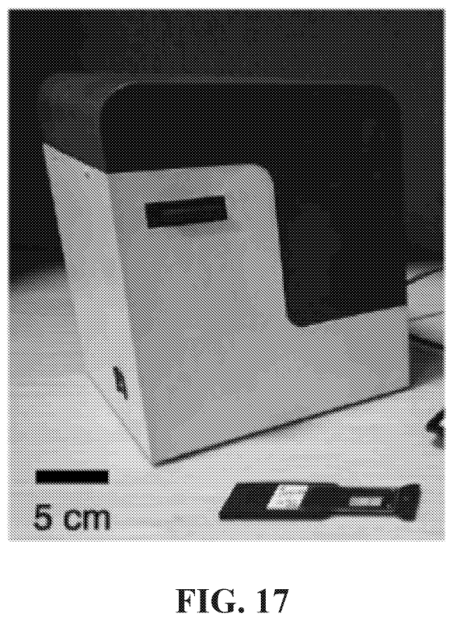

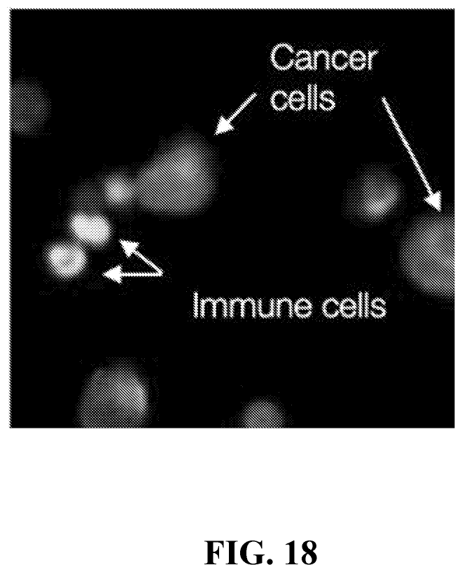

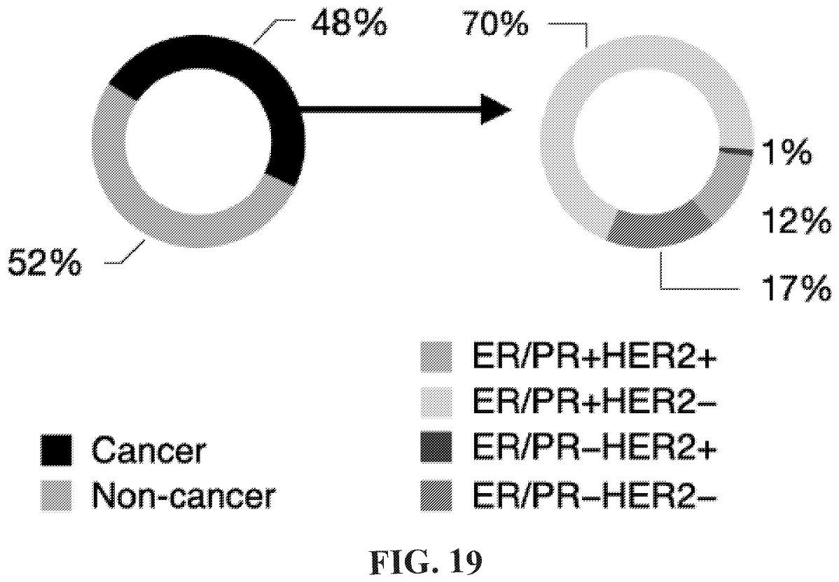

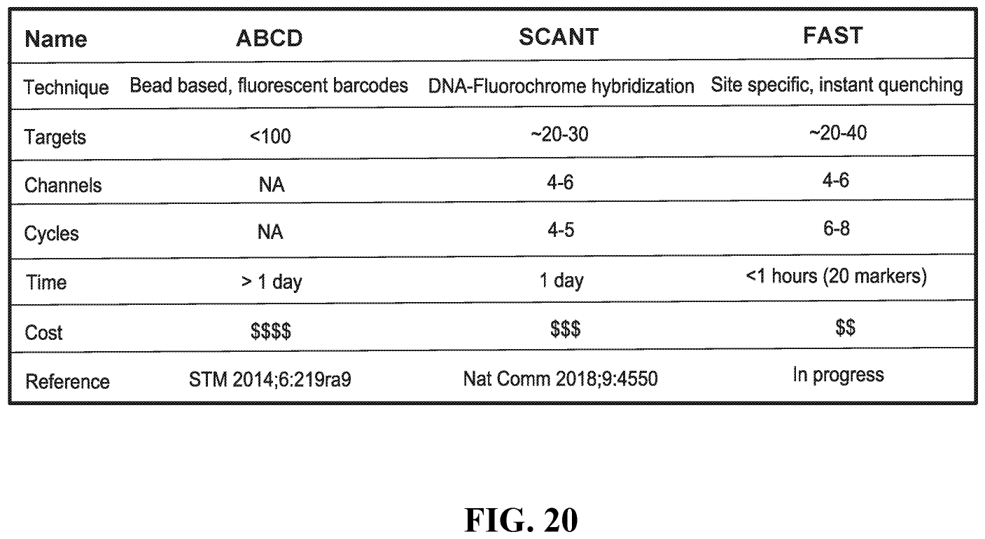

For simultaneous multi-color (≥4) cellular analyses, Cytometry Portable Analyzer (CytoPAN) can be used. The system was originally built for operation in remote locations (FIG. 17) but has additional applications in POC settings (OR, interventional suites, doctors' offices). The excitation light sources were positioned for side illumination through a glass slide, and a single emission filter with four pass bands was used. No dichroic mirrors or mechanical filter changes were necessary. Furthermore, intelligent software streamlined the entire assay, including light-source calibration, sample slide detection, data acquisition and cellular analyses. CytoPAN had four different fluorescent channels (FIG. 18) and a bright-field imaging capacity. Automated algorithms profiled analyzed individual cells and produced summary reports for cancer diagnosis (FIG. 19). This affordable system (<$1,000), in which the compounds and methods of the present application are implemented, is operable by non-skilled workers.

The fluorescent systems discussed above are still bound by the physical SBP limit and there thus remains a trade-off between FOV and spatial resolution. Computational methods used in coherent imaging cannot be applied, because fluorescent emission does not carry phase information. A straightforward workaround is to combine sample scanning with miniaturized optics; a key technical requirement is to automate such operations including stage movement and imaging stitching. Equally important is the development of tools for reliable sample preparation, for example by connecting fluidic cartridges with cost-effective pumping systems. This would speed up assays and minimize procedural errors particularly in cyclic imaging, which requires repeated fluidic handling such as quenching, washing, and labeling.

### CONCLUSION

In contemporary laboratory medicine, virtually all blood and urine tests have been automated to reduce cost, improve test quality and accommodate the increasing volume of clinical samples. The methods disclosed here allow for automation to be applicable for FNA analysis of cancer samples, particularly in resource-limited environments. Suitable example includes automated POC cytometry, including the rigorous evaluation of cellular markers, staining techniques and kit developments. Automated, AI-based diagnostic DNA-karyometry is another suitable application. Also automated image cytometry, molecularly testing cytology samples, and fluorescent in situ hybridization (FISH) for EGFR, KRAS and BRAF mutation and other cytogenetic abnormalities should be feasible with appropriate amplification strategies. Finally, the compounds and methods of the present disclosure provide the techniques for analyzing FNA specimens for disease (e.g., cancer) diagnosis and monitoring. Inexpensive automated cellular analyses and molecular testing may be contemplated for organ FNA obtained from liver, kidney or blood/bone marrow.

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “carboxy” refers to a —C(O)OH group.

As used herein, “halo” refers to F, Cl, Br, or I. In some embodiments, a halo is F, Cl, or Br. The term “perhalo-” (such as “perfluoro-”) refers to groups where each H atom in the group is replaced with a halogen.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphtyl. The term “arylene” refers to a divalent aryl group, such as a phenylene. The term “arylene” refers to a divalent aryl group.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

### EXAMPLES

### Example 1—Preparation of Fluorescent Probes

### Preparation of BCN-FAST Linker Compound (1):

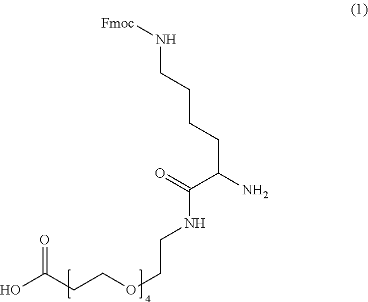

Prepared as described in Ko, J., Oh, J., Ahmed, M. S., Carlson, J. C. T. & Weissleder, R. Ultra-fast Cycling for Multiplexed Cellular Fluorescence Imaging, Angew Chem Int Ed Engl, 59, 6839-6846 (2020), which is incorporated herein by reference in its entirety.

### Preparation of BCN-FAST Linker Compound (2):

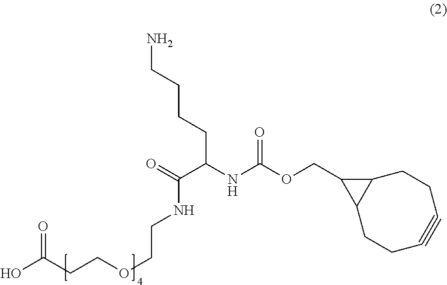

To an aliquot of compound 1 (12 mg, 19.5 μmoles) dissolved in 300 μL of dry DMSO were added DIPEA (2 eq., 11.95 μL) and 10 mg of (1R,8S,9s)-bicyclo[6.1.0]non-4-yn-9-ylmethyl N-succinimidyl carbonate (BCN-NHS, 1.75 eq., 34.3 μmol). The reaction mixture was vortexed assertively in an Eppendorf tube. After 5 minutes, complete conversion was observed by LCMS and 45 μL of piperidine were added for Fmoc deprotection. After a further five minutes, the reaction mixture was injected directly onto a 25 g SNAP Bio C₁₈ column (Biotage) and purified with an ammonium formate (pH 8.5):acetonitrile gradient. The collected fractions were rotovapped, dissolved in 100 μL of DMSO, and then desalted with a Waters tC18 Sep Pak, eluted with methanol, and evaporated to dryness, yielding 7.2 mg of 2.

### Preparation of BCN-FAST 488 Compound (3):

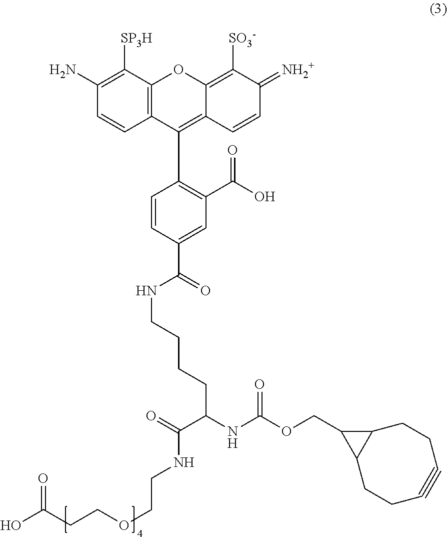

To a solution of AlexaFluor 488-TFP (2.5 mg, 3.66 μmol, purchased from Fluoroprobes (USA)) dissolved in dry DMSO were added 2.88 mg of compound 2 (1.4 eq., 5 μmoles) and DIPEA (2.5 eq., 1.6 μL). The reaction mixture was vortexed to mix and shielded from light. After 5 minutes, LCMS indicated complete consumption of the AF488-TFP, so the reaction mixture was injected directly onto a 10 g SNAP Bio C₁₈ column (Biotage) and purified with an ammonium formate (pH 8.5):acetonitrile gradient. Fractions containing pure compound 3 were collected and rotovapped to dryness with shielding from ambient light.

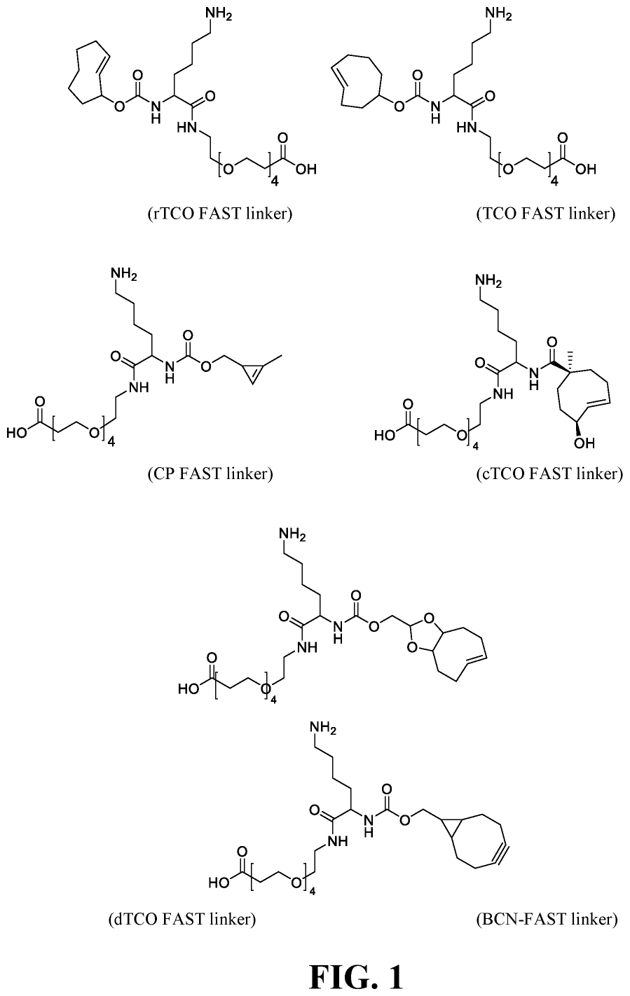

AF488-containing probes containing rTCO, TCO, CP, cTCO, and dTCO were prepared in a similar manner staring from compound 1 and derivatizing with the corresponding activated dienophile starting material (See FIG. 1) to prepare the respective analogs of compound 2. Other fluorophore-based probes (such as AF647-based probes) were prepared using same or similar protocols and commercially available starting materials.

### Example 2—Probe Stability and Quenching Kinetics

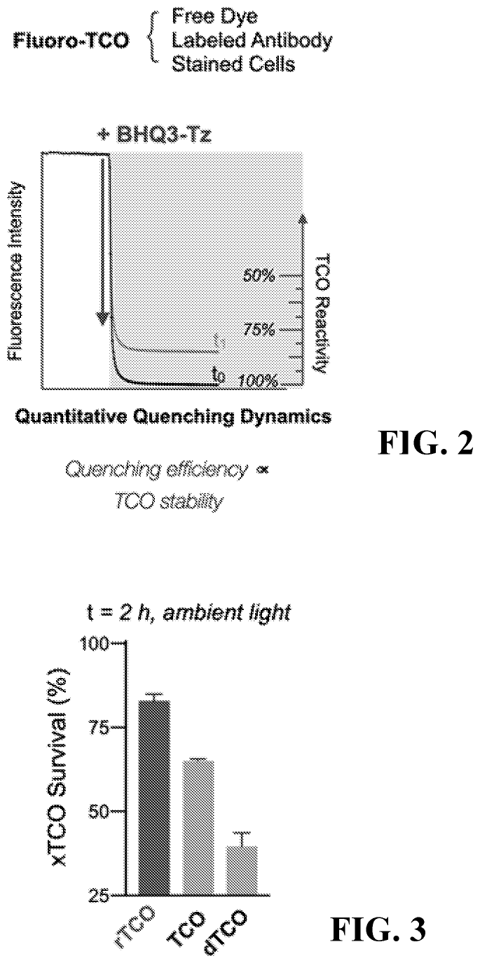

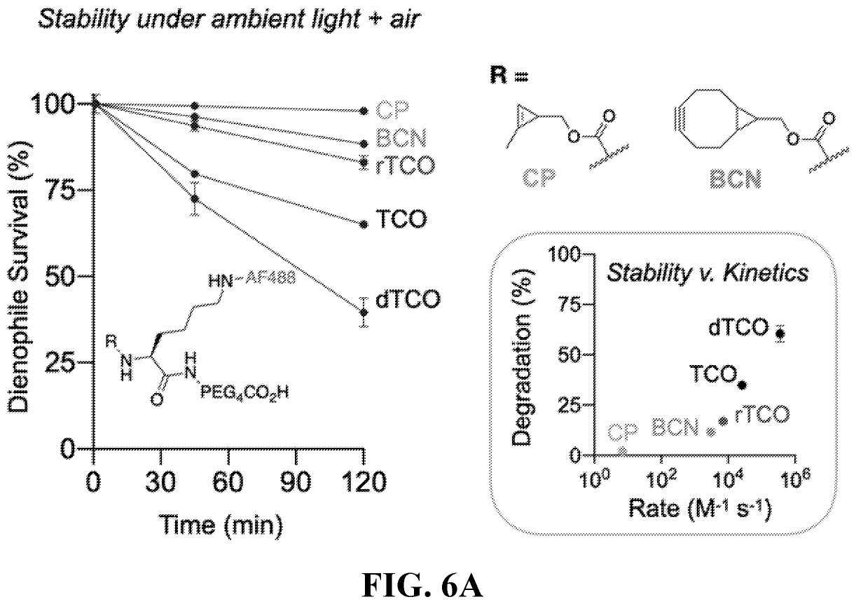

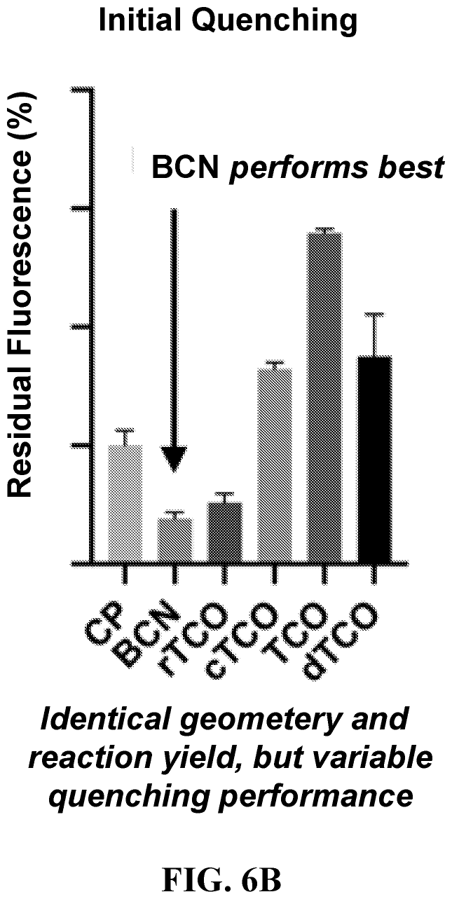

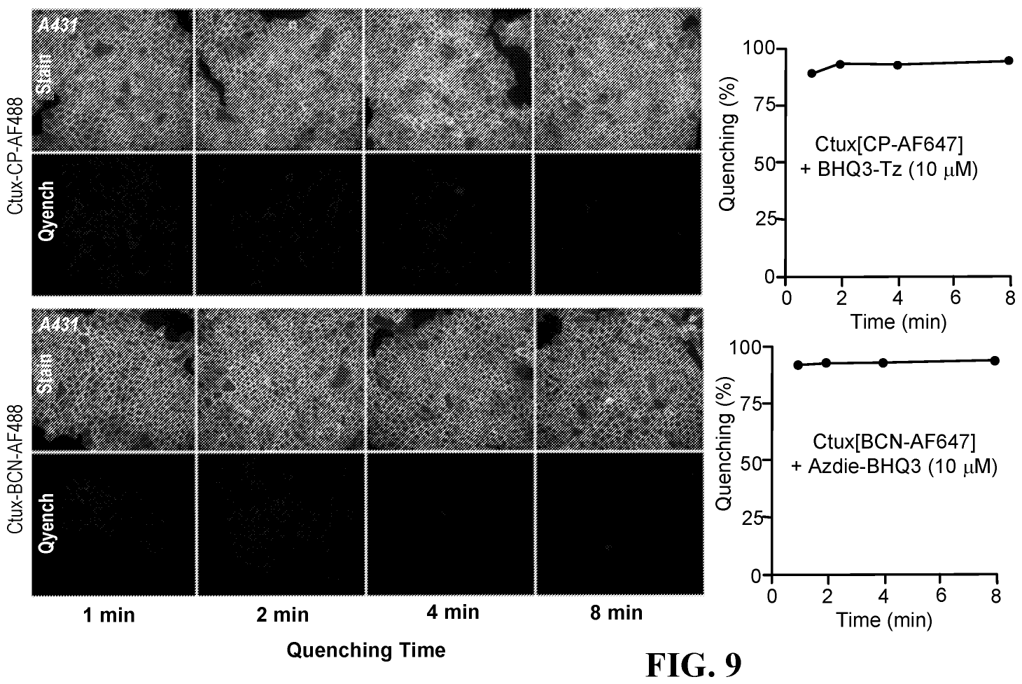

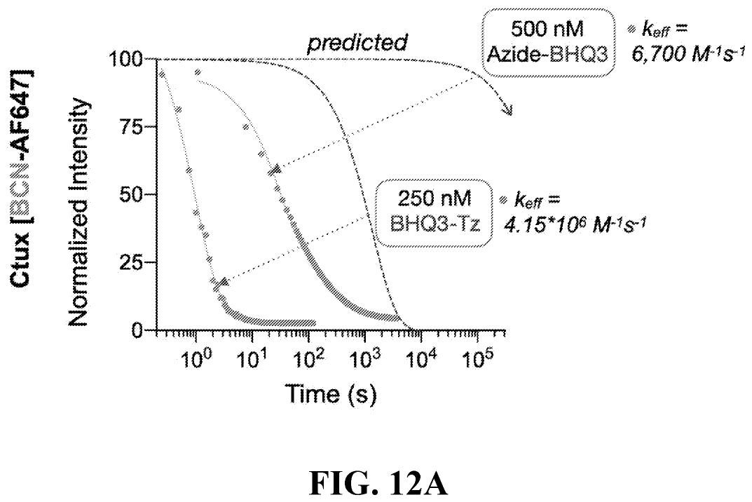

Stability of the fluorescent probes prepared in Example 1 under ambient light and oxygen, as well as kinetics and efficiency of their quenching with BHQ3 quencher (including quenching fluorescence on the cell surfaces stained with dienophile- and fluorophore-labeled antibodies) are shown in FIGS. 2-12A.

### General Methods

### FAST-AF488 Probe Stability Measurements

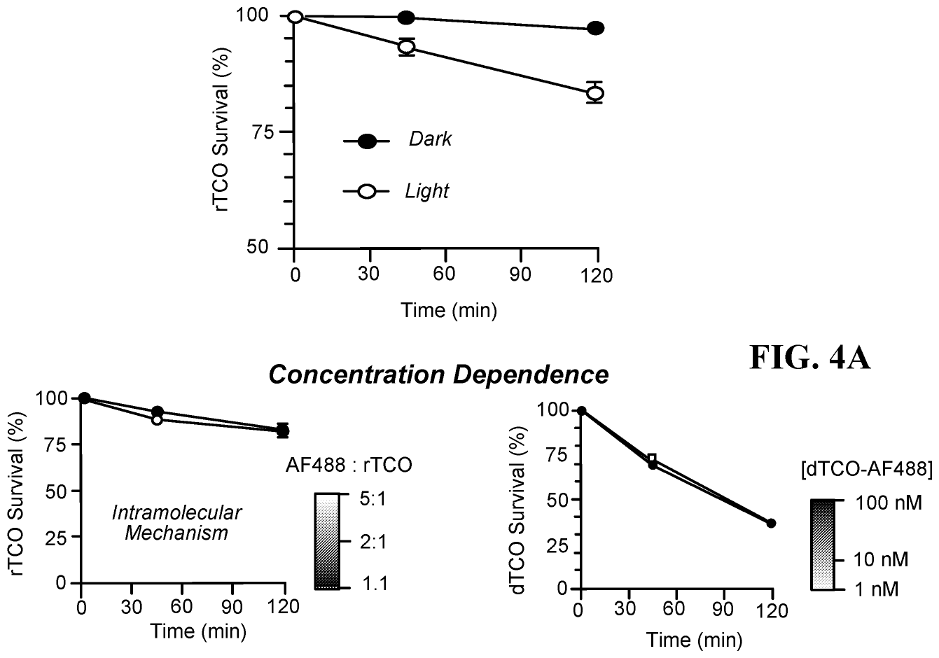

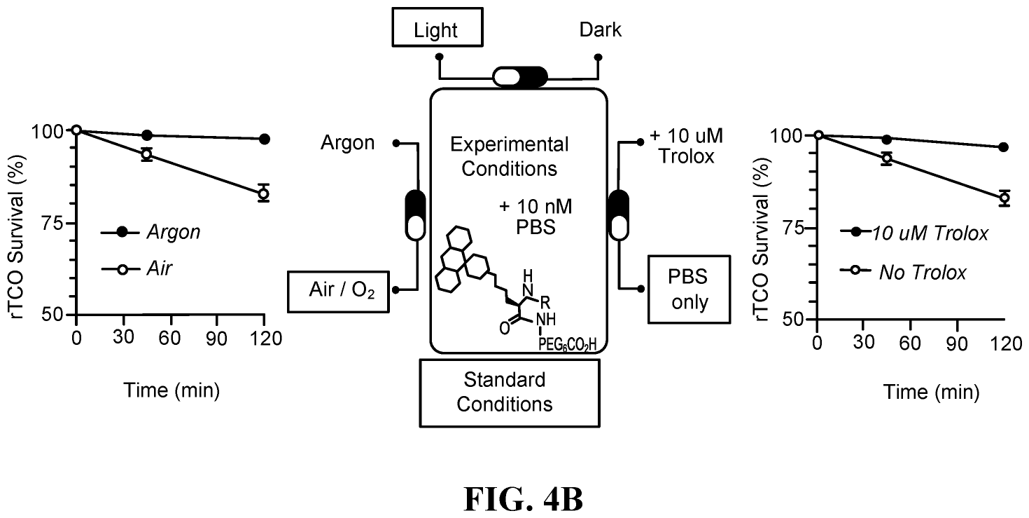

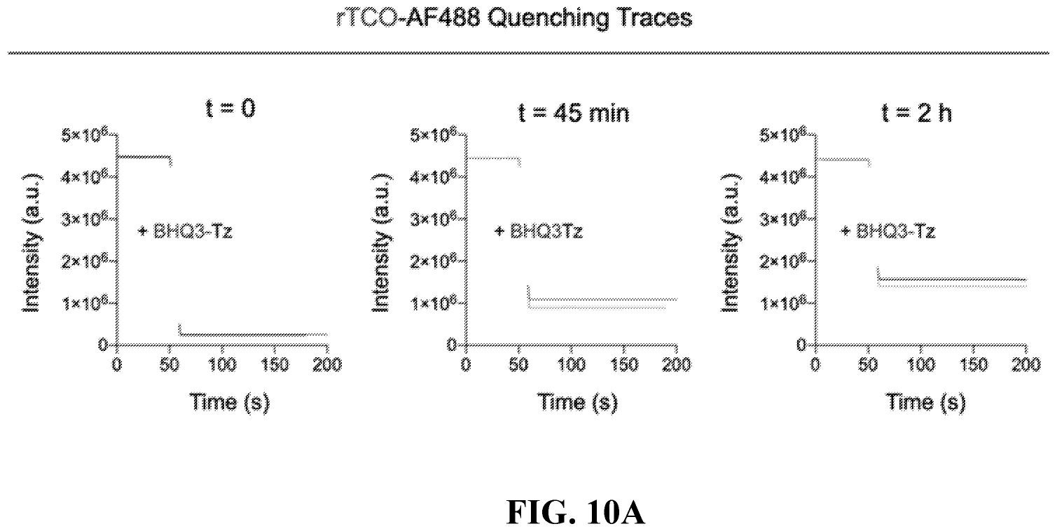

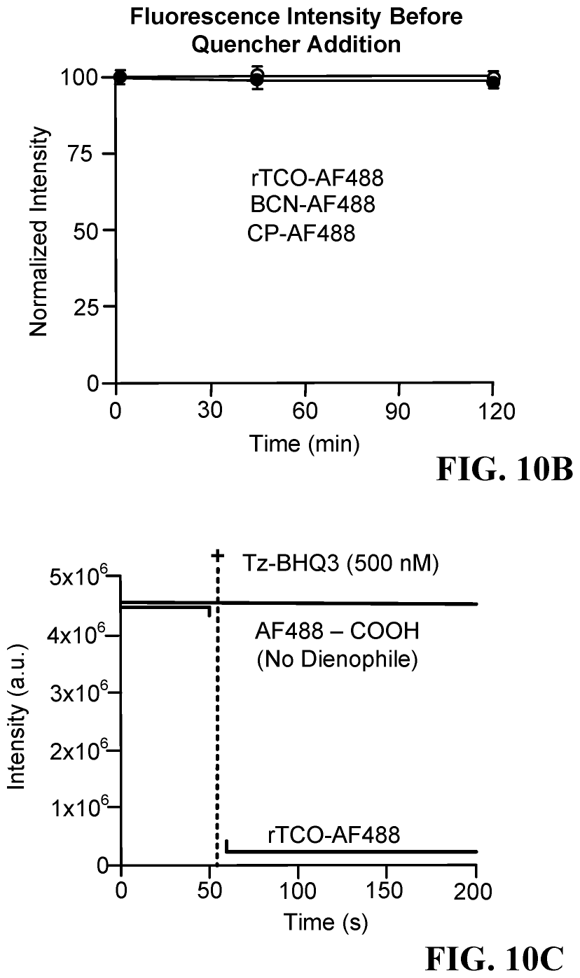

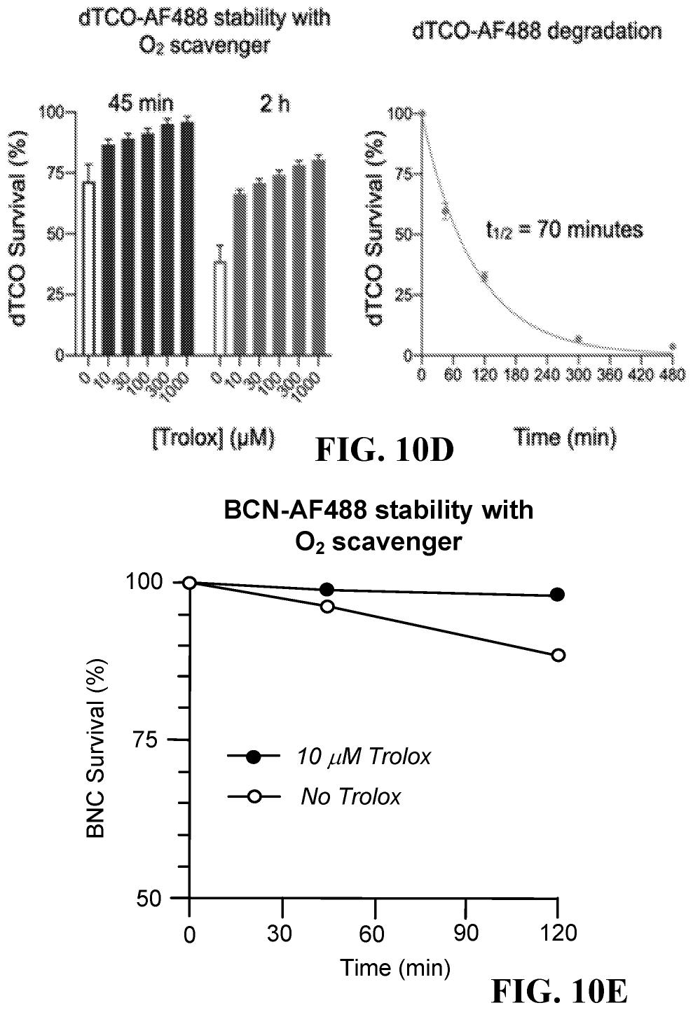

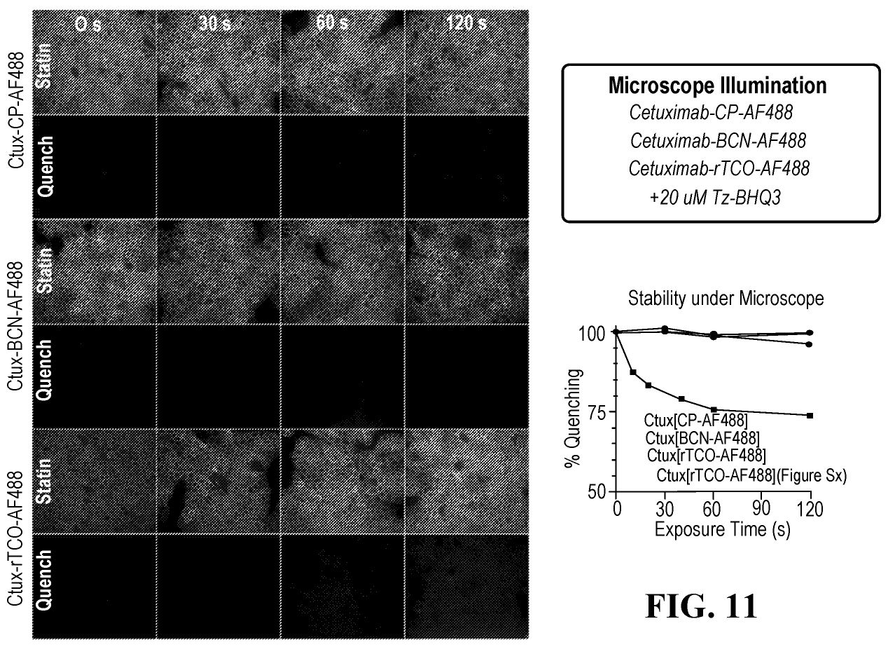

Stock solutions of FAST-AF488 probes (prepared in Example 1) in DMSO (1 mM) were diluted into PBS to a concentration of 10 nM with the addition of 0-1000 μM VectaCell Trolox (Vector Laboratories). 5 mL aliquots were added to a 15 mL glass vial and left open to air under a Philips F32T8/TL735 700 Series 32 Watt fluorescent light bulb in standard 6′ chemical fume hood or kept in the dark. The solution was then transferred to a quartz cuvette for fluorescence quenching measurements. Quenching efficiency was measured using a time-based fluorescence acquisition at the appropriate dye-specific wavelengths. After measuring the initial fluorescence signal, Tz-BHQ3 (500 nM) was added to the cuvette and the fluorescence signal was measured again once the quenching reaction was complete (FIG. 10A). Quenching performance overtime was determined for each probe relative to the initial quenching efficiency at t=0. For measurements under argon, a plastic glove bag was evacuated, backfilled, and purged with argon gas before open glass vials containing the FAST-AF488 solutions were placed inside and left under the same fluorescent light bulbs. See FIGS. 4A and 4B.

### LCMS Degradation Characterization

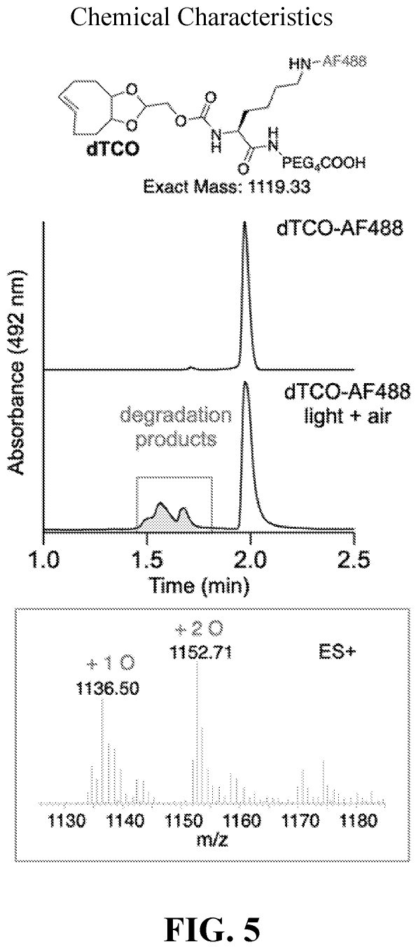

dTCO-AF488 probe was diluted into water to a concentration of 3 μM. Two 5 mL aliquots were added to 15 mL glass vials and left open to air under laboratory lights as described above for two hours. The vials were combined and concentrated by rotary evaporation and redissolved at a concentration of 150 μM for LCMS injection. See FIG. 5.

### Antibody Modification

FAST-probes were converted to the NHS ester following the previously published TSTU-ENBA NHS activation method. Cetuximab (2 mg/mL) was buffer-exchanged with Zeba spin desalting columns (40 K MWCO) into a PBS-bicarbonate solution (100 mM sodium bicarbonate in PBS, pH 8.4) then incubated with 10-20 equivalents of the activated NHS-FAST-probe for 25 minutes at room temperature. After this, excess fluorophore was removed with another Zeba spin desalting column (40 K MWCO) into PBS. The degree of labeling (DOL) of the conjugated antibody was determined by measuring the absorbance spectrum on a Nanodrop 1000 using the appropriate extinction coefficients and correction factors for the antibody and dyes. The conjugated antibodies were stored at 4° C. in the dark until used.

### Quenching Kinetics of FAST-Antibodies

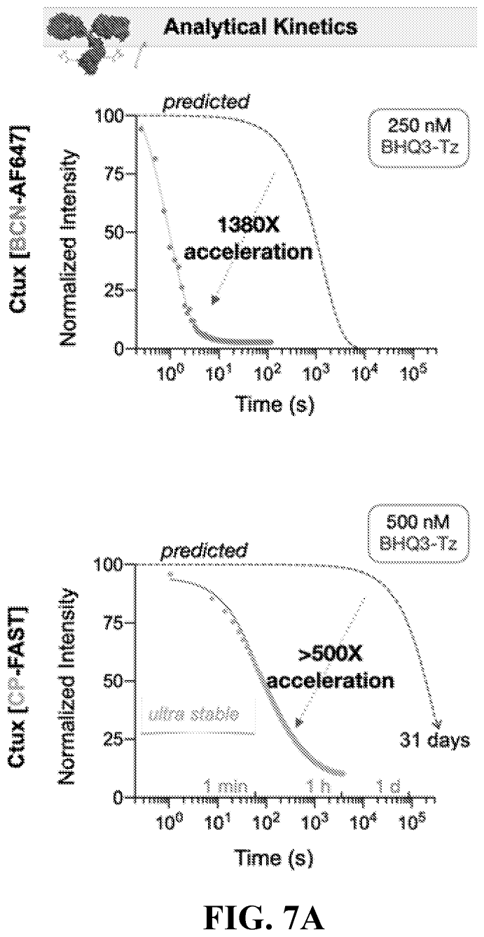

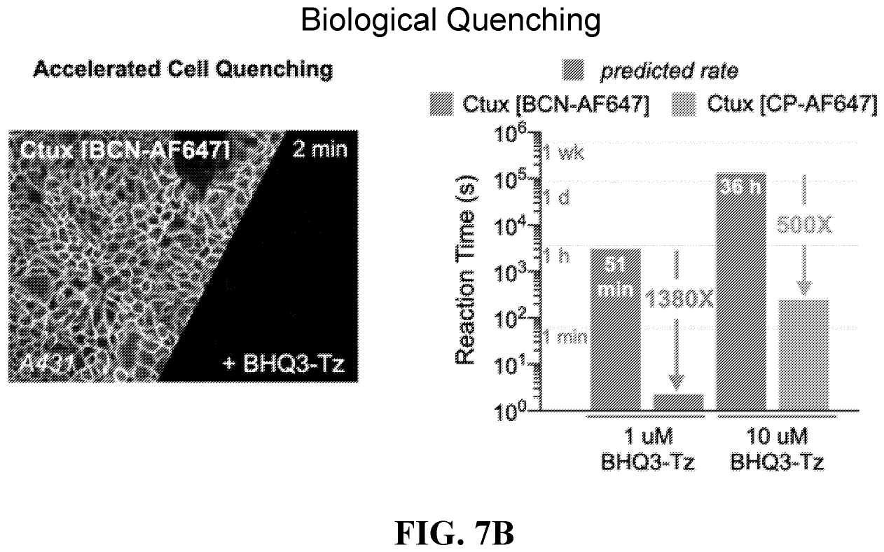

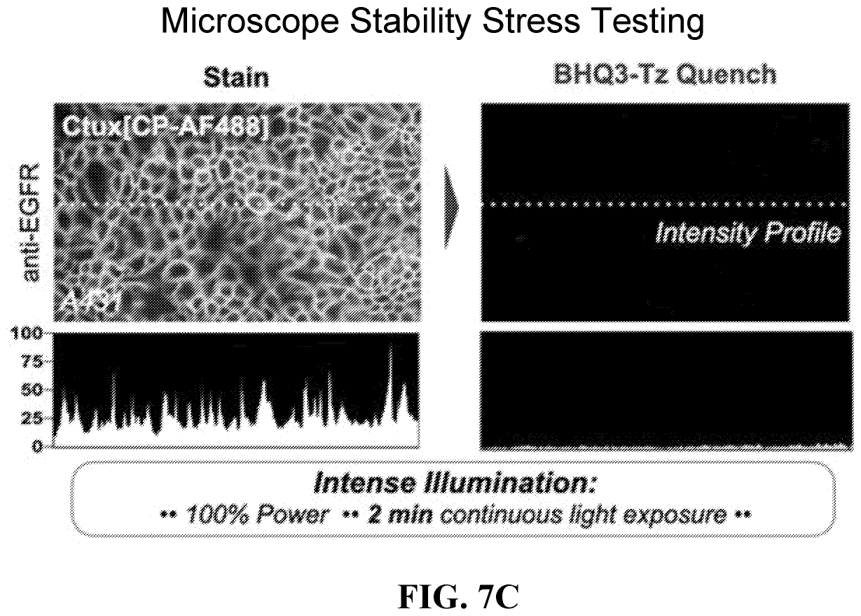

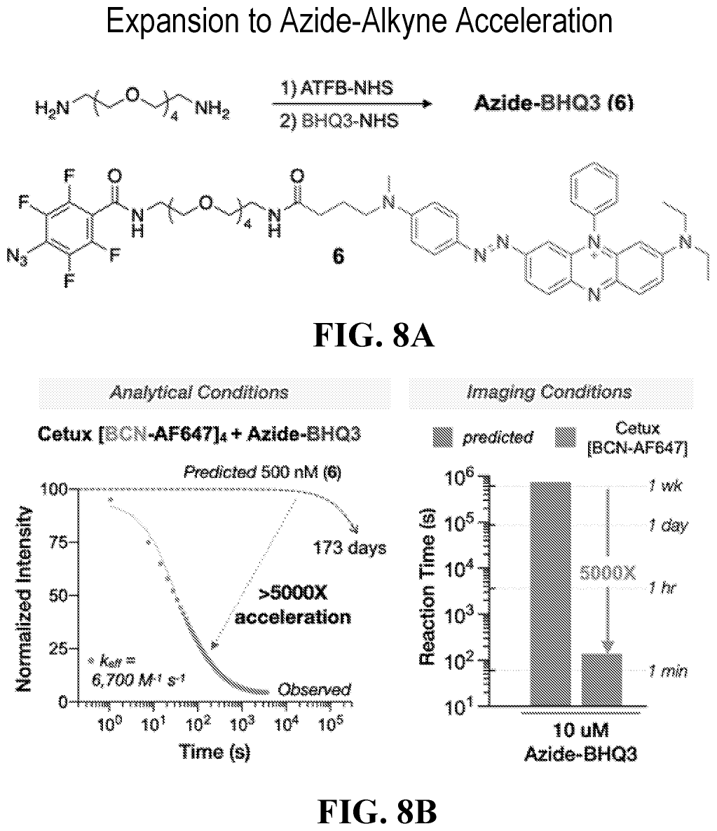

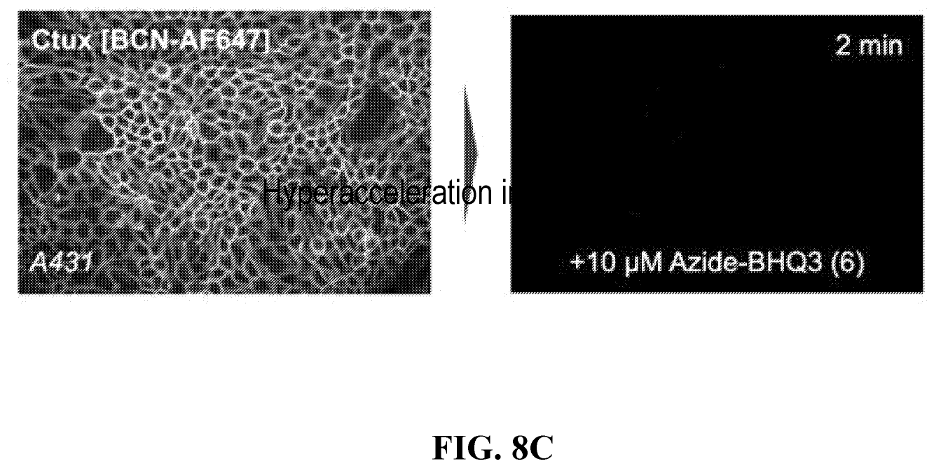

CP or BCN FAST-labeled antibodies were stored in PBS at 4° C. at concentrations of 5-15 μM after labeling. Disposable polystyrene cuvettes were blocked with 2 mL 1% BSA in PBS which was then removed and replaced with a 0.01% BSA solution in PBS to reduce nonspecific adsorption of the antibodies. Time-based fluorescence acquisitions at the appropriate dye-specific wavelengths were initiated, and the baseline emission of the buffer solutions measured. FAST-labeled antibodies were diluted into the blocked cuvette to a concentration of 4-10 nM and after measuring initial fluorescence, 10-20 μL of either Tz-BHQ3 or Azide-BHQ3 were added via the instrument's sample addition port and data acquisition continued until the quenching reaction was complete. See FIGS. 7A, 8B.

### Kinetic Fitting

Data were analyzed in GraphPad Prism 9 (Graphpad Software). For FAST-labeled antibodies, curves were fitted to a double exponential (two phase) decay with the time of BHQ3 addition set to t=0 for fitting purposes. Rate constants are reported in the figures.

### Stopped-Flow Click Kinetics

The unaccelerated reaction rate of BCN-PEG2-amine with benzylamino-tetrazine was measured on a stopped flow spectrophotometer as previously published (Carlson, J. C. T., et al., Unraveling Tetrazine-Triggered Bioorthogonal Elimination Enables Chemical Tools for Ultrafast Release and Universal Cleavage. J Am Chem Soc 140, 3603-3612 (2018)). Data were analyzed in GraphPad Prism 9 (Graphpad Software) and second order rate constants were calculated from the second order rate equation (Kinetics and Mechanism) using the nonlinear fits of the absorbance vs. time curves.

### Cell Culture

A431 cells were purchased from the American Tissue Culture Collection (ATCC). A431 cells were passaged in DMEM (10% FBS, 1% penicillin/streptomycin) according to the specifications from ATCC. Cells were first grown in a 150 mm cell culture dish and then seeded on Millicell 8-well EZ slides (Millipore) for imaging. After 48 hours, confluency was assessed and cells were fixed with 4% paraformaldehyde in PBS (10 min) and stored at 4° C. until imaging.

### Immunostaining and Quenching

Fixed A431 cells were stained with 5 μg/ml modified antibodies (Cetuximab-TCO/rTCO-AF488, Cetuximab-BCN/CP-AF488/AF647) for 15 mins at room temperature in the dark. For microscope illumination, cells were exposed to microscope fluorescent light prior to quenching. For quenching, 10 μM Tz-BHQ3 was used with different incubation times (1, 2, 4, 8 min) in PBS-bicarb (pH 9), followed by three washes to remove free Tz-BHQ3. For Trolox addition, cells were imaged in PBS with different Trolox concentrations (0, 50, 250 μM), followed by microscope illumination of 120 s exposure. After illumination, cells were quenched and imaged to quantify quenching efficiency.

### Fluorescent Imaging and Analysis

An Olympus BX-63 upright automated epifluorescence microscope was used to acquire fluorescent images. FITC and Cy5 filter cubes were used to excite AF488 and AF647 fluorophores respectively. ImageJ was used to measure fluorescent intensities of cells. Quenching efficiency was calculated by the following equation. Residual MFI=(quenched MFI−background MFI)/(stained MFI−background MFI).

### Numbered Paragraphs

In some embodiments, the invention provided in this document can be described by reference to the following numbered paragraphs:
Paragraph 1. A compound of Formula (I):

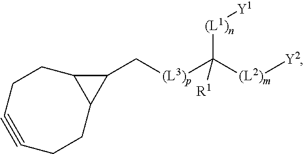

or a pharmaceutically acceptable salt thereof, wherein:
R¹ is selected from H, C₁₋₃ alkyl, and C₁₋₃ haloalkyl;
each L¹ is independently selected from N(R^{N}), O, C(═O), S(═O)₂, C₁₋₆ alkylene, —(OCH₂CH₂)ₓ—, and —(CH₂CH₂O)ₓ—;
n is an integer from 1 to 10;
each L² is independently selected from N(R^{N}), O, C(═O), S(═O)₂, C₁₋₆ alkylene, —(OCH₂CH₂)ₓ—, and —(CH₂CH₂O)ₓ—;
m is an integer from 1 to 10;
each L³ is independently selected from N(R^{N}), O, C(═O), S(═O)₂, C₁₋₆ alkylene, —(OCH₂CH₂)ₓ—, and —(CH₂CH₂O)ₓ—;
wherein each of said C₁₋₆ alkylene groups within L¹, L², or L³ is optionally substituted with (L⁴)ₒ-Y³;
each R^{N} is independently selected from H, C₁₋₃ alkyl, C₁₋₃ haloalkyl, and (L⁴)ₒ-Y³;
each L⁴ is independently selected from N(R^{N1}), O, C(═O), S(═O)₂, C₁₋₆ alkylene, —(OCH₂CH₂)ₓ—, and —(CH₂CH₂O)ₓ—;
each R^{N1} is independently selected from H, C₁₋₃ alkyl, and C₁₋₃ haloalkyl;
p is an integer from 1 to 20;
o is an integer from 1 to 10;
each x is independently an integer from 1 to 2,000;
each Y³ is independently selected from a moiety of formula (i) and a moiety of formula (ii):

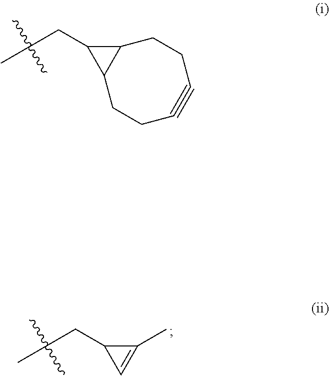

Y¹ is selected from NR^{c1}R^{1A}, OR², and C(═O)R³;
R^{1A} selected from H, an amine protecting group, and a fluorophore;
R² is selected from H, an alcohol protecting group, and a fluorophore;
R³ is selected from OR^{a1} and a fluorophore;
Y² is selected from C(═O)OR^{a1}, NR^{c1}R⁴, OR⁵; and a group reactive with a side chain of an amino acid of a protein;
R^{a1} is selected from H and a carboxylic acid protecting group;
R^{c1} is selected from H, C₁₋₃ alkyl, and C₁₋₃ haloalkyl;
R⁴ is selected from H and an amine protecting group; and
R⁵ is selected from H and an alcohol protecting group.

Paragraph 2. The compound of paragraph 1, R¹ is H.

Paragraph 3. The compound of paragraph 1 or 2, wherein n is an integer from 1 to 5, and each L¹ is selected from NH, O, C(═O), and C₁₋₆ alkylene.

Paragraph 4. The compound of paragraph 3, wherein m is an integer from 1 to 5, and each L² is independently selected from NH, C(═O), C₁₋₆ alkylene, —(OCH₂CH₂)ₓ—, and —(CH₂CH₂O)ₓ—.

Paragraph 5. The compound of paragraph 4, wherein m is 5.

Paragraph 6. The compound of any one of paragraphs 1-5, wherein x is an integer from 1 to 10.

Paragraph 7. The compound of paragraph 1, wherein:
R¹ is H;
n is an integer from 1 to 5, and each L¹ is selected from NH, O, C(═O), and C₁₋₆ alkylene;
m is an integer from 1 to 5, and each L² is independently selected from NH, O, C(═O), C₁₋₆ alkylene, —(OCH₂CH₂)ₓ—, and —(CH₂CH₂O)ₓ—; and
x is an integer from 2 to 10.

Paragraph 8. The compound of any one of paragraphs 1-7, wherein:
p is an integer from 1 to 5,
each L³ is selected from NR^{N}, O, C(═O), and C₁₋₆ alkylene,
said C₁₋₆ alkylene is optionally substituted with (L⁴)ₒ-Y³; and
R^{N} is selected from H, C₁₋₃ alkyl, and (L⁴)₀-Y³.

Paragraph 9. The compound of any one of paragraphs 1-8, wherein each L³ is selected from NH, O, C(═O), and C₁₋₆ alkylene, which is optionally substituted with (L⁴)ₒ-Y³.

Paragraph 10. The compound of paragraph 9, having formula:

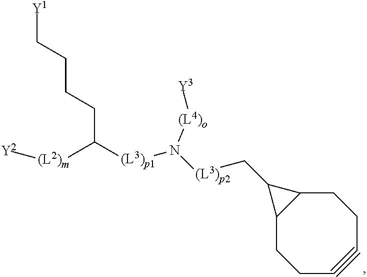

or a pharmaceutically acceptable salt thereof, wherein the sum of p1 and p2 is less than p by at least 1.

Paragraph 11. The compound of any one of paragraphs 1-8, wherein:
each L³ is selected from NR^{N}, O, C(═O), and C₁₋₆ alkylene, and
R^{N} is selected from H, C₁₋₃ alkyl, and (L⁴)ₒ-Y³.

Paragraph 12. The compound of paragraph 11, having formula:

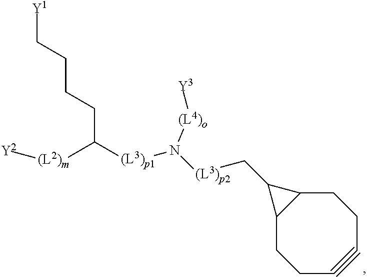

or a pharmaceutically acceptable salt thereof, wherein the sum of p1 and p2 is less than p by at least 1.

Paragraph 13. The compound of any one of paragraphs 1-12, wherein o is an integer from 1 to 5 and each L⁴ is independently selected from NH, O, C(═O), and C₁₋₆ alkylene.

Paragraph 14. The compound of any one of paragraphs 1-13, wherein Y³ is a moiety of formula (i):

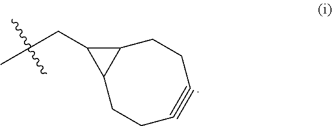 [SMILES: *CC1C2CCC#CCCC12]

Paragraph 15. The compound of any one of paragraphs 1-13, wherein Y³ is a moiety of formula (ii):

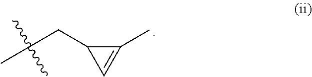 [SMILES: *CC1C=C1C]

Paragraph 16. The compound of any one of paragraphs 1-8, wherein each L³ is selected from NR^{N}, O, C(═O), and C₁₋₆ alkylene.

Paragraph 17. The compound of paragraph 16, wherein:
n is 1 and L¹ is C₁₋₆ alkylene;
m is 4, and each L² is independently selected from NH, C(═O), C₁₋₆ alkylene, —(OCH₂CH₂)ₓ—, and —(CH₂CH₂O)ₓ—; and
p is 3, and each L³ is independently selected from NH, O, and C(═O).

Paragraph 18. The compound of paragraph 17, wherein Formula (I) has formula:

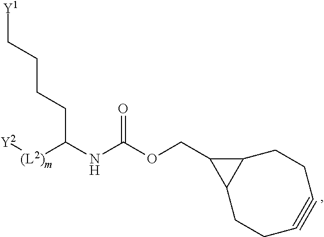

or a pharmaceutically acceptable salt thereof.

Paragraph 19. The compound of paragraph 17, wherein Formula (I) has formula:

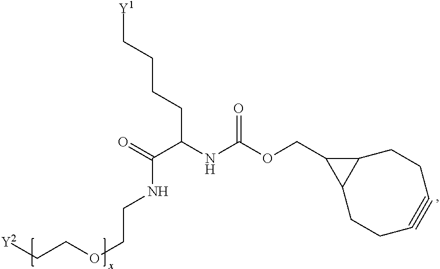

or a pharmaceutically acceptable salt thereof.

Paragraph 20. The compound of any one of paragraphs 1-19, wherein:
Y¹ is NHR^{1A}; and
Y² is selected from C(═O)OR″ and a group reactive with a side chain of an amino acid of a protein.

Paragraph 21. The compound of any one of paragraphs 1-19, wherein:
Y¹ is NH₂; and
Y² is C(═O)OH.

Paragraph 22. The compound of any one of paragraphs 1-19, wherein:
Y¹ is NHR^{1A};
R¹ is an amine protecting group; and
Y² is C(═O)OH.

Paragraph 23. The compound of any one of paragraphs 1-19, wherein:
Y¹ is NHR^{1A};
R¹ is a fluorophore; and
Y² is C(═O)OH.

Paragraph 24. The compound of any one of paragraphs 1-19, wherein:
Y¹ is NHR^{1A};
R¹ is a fluorophore;
Y² is C(═O)OR^{a1}; and
R^{a1} is a carboxylic acid protecting group.

Paragraph 25. The compound of any one of paragraphs 1-19, wherein:
Y¹ is NHR^{1A};
R¹ is a fluorophore; and
Y² is a group reactive with a side chain of an amino acid of a protein.

Paragraph 26. The compound of paragraph 1, wherein the compound of Formula (I) is selected from any one of the following compounds:

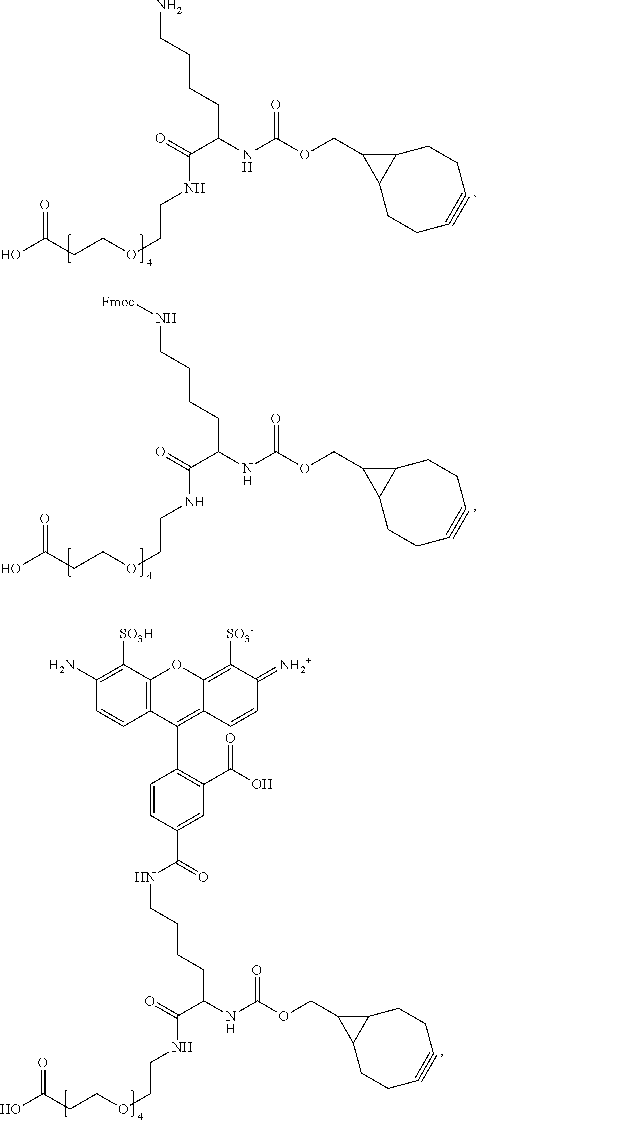

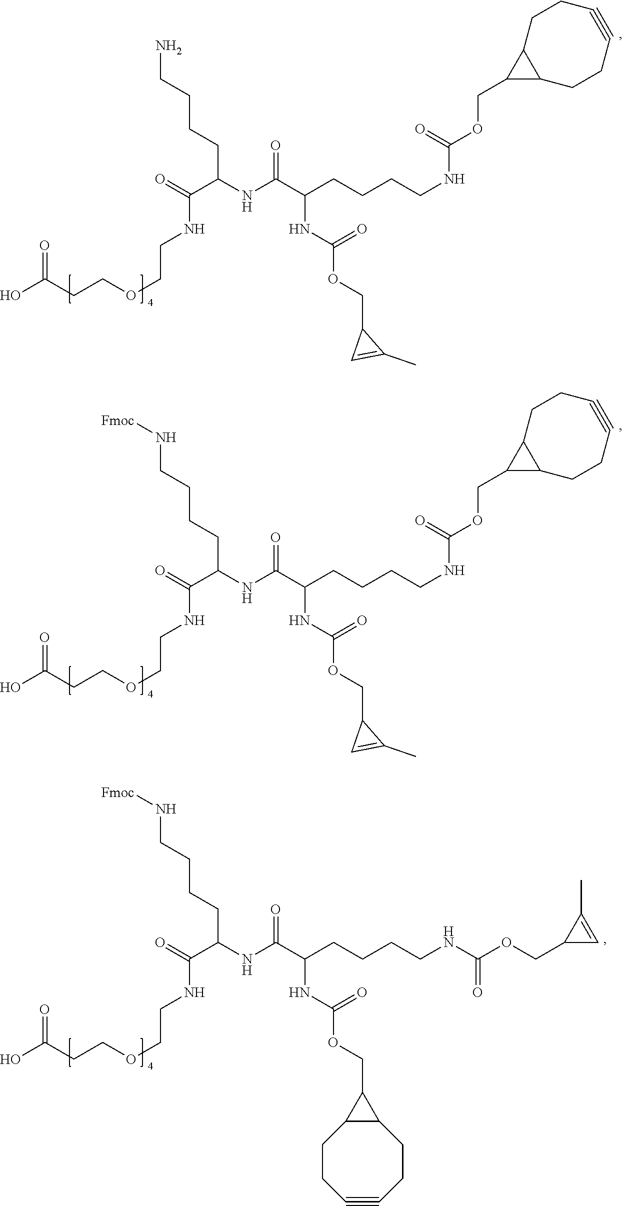

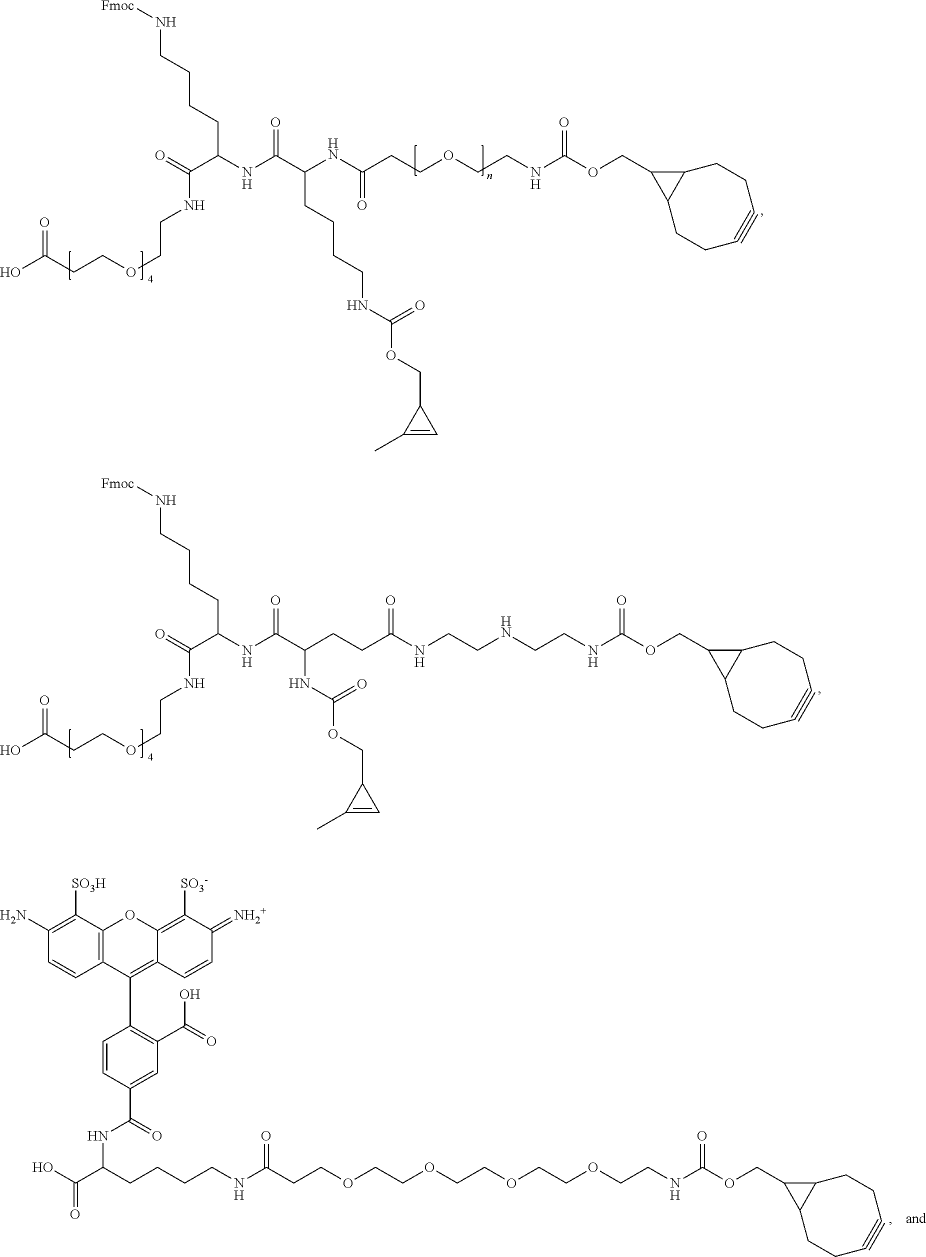

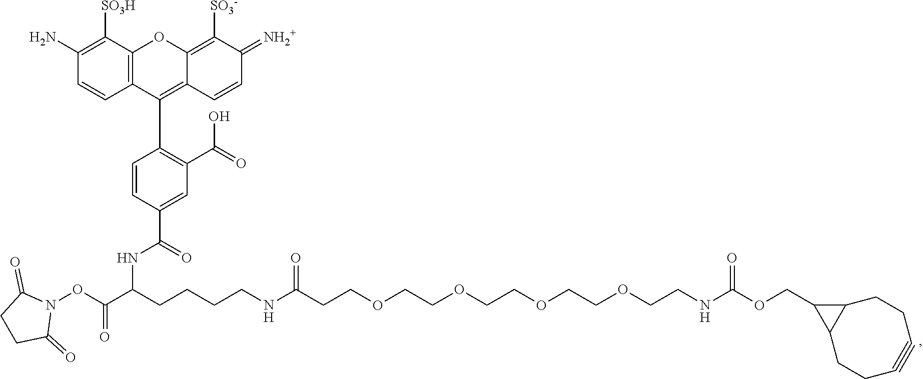

or a pharmaceutically acceptable salt thereof.

Paragraph 27. A protein conjugate of Formula (II):

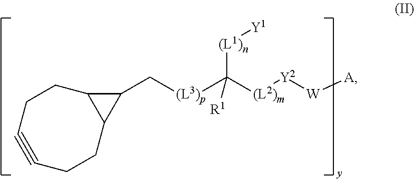

or a pharmaceutically acceptable salt thereof, wherein:
A is a protein;
y is an integer from 1 to 10;
R¹ is selected from H, C₁₋₃ alkyl, and C₁₋₃ haloalkyl;
each L¹ is independently selected from N(R^{N}), O, C(═O), S(═O)₂, C₁₋₆ alkylene, —(OCH₂CH₂)ₓ—, and —(CH₂CH₂O)ₓ—;
n is an integer from 1 to 10;
each L² is independently selected from N(R^{N}), O, C(═O), S(═O)₂, C₁₋₆ alkylene, —(OCH₂CH₂)ₓ—, and —(CH₂CH₂O)ₓ—;
m is an integer from 1 to 10;
each L³ is independently selected from N(R^{N}), O, C(═O), S(═O)₂, C₁₋₆ alkylene, —(OCH₂CH₂)ₓ—, and —(CH₂CH₂O)ₓ—;
wherein each of said C₁₋₆ alkylene groups within L¹, L², or L³ is optionally substituted with (L⁴)ₒ-Y³;
each R^{N} is independently selected from H, C₁₋₃ alkyl, C₁₋₃ haloalkyl, and (L⁴)ₒ-Y³;
each L⁴ is independently selected from N(R^{N1}), O, C(═O), S(═O)₂, C₁₋₆ alkylene, —(OCH₂CH₂)ₓ—, and —(CH₂CH₂O)ₓ—;
each R^{N1} is independently selected from H, C₁₋₃ alkyl, and C₁₋₃ haloalkyl;
p is an integer from 1 to 20;
o is an integer from 1 to 10;
each x is independently an integer from 1 to 2,000;
each Y³ is independently selected from a moiety of formula (i) and a moiety of formula (ii):

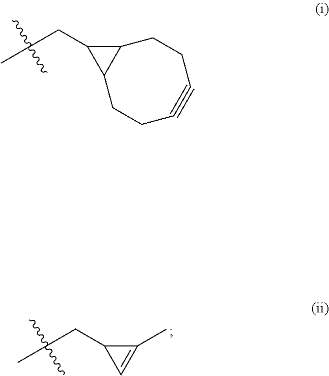

Y¹ is selected from NR^{c1}R^{1A}, OR², and C(═O)R³;
R^{1A}, R², and R³ are each independently selected from a fluorophore;
R^{c1} is selected from H, C₁₋₃ alkyl, and C₁₋₃ haloalkyl;
each W is selected from:
(i) O of a side chain of serine, threonine, or tyrosine of the protein A;
(ii) S of a side chain of cysteine of the protein A;
(iii) NH of a side chain of lysine of the protein A; and
(iv) C(═O) of a side chain of aspartic acid or glutamic acid of the protein A; and
Y² is a residue of a group which, prior to conjugation with the protein A, was a group reactive with a side chain of an amino acid of the protein A.

Paragraph 28. The conjugate of paragraph 27, wherein the protein is selected from an antibody, an antibody fragment, an engineered antibody, a peptide, and an aptamer.

Paragraph 29. The conjugate of paragraph 28, wherein the antibody is specific to an antigen which is a biomarker of a disease or condition.

Paragraph 30. The conjugate of paragraph 29, wherein the disease or condition is cancer.

Paragraph 31. The conjugate of any one of paragraphs 27-30, wherein y is an integer from 4 to 6.

Paragraph 32. The conjugate of any one of paragraphs 27-31, wherein each Y² is C(═O) and at least one W is NH of a side chain of lysine of the protein A.

Paragraph 33. The conjugate of any one of paragraphs 27-31, wherein each Y² is C(═O) and at least one W is S of a side chain of cysteine of the protein A.

Paragraph 34. The conjugate of any one of paragraphs 27-33, wherein Y¹ is NHR^{1A}.

Paragraph 35. The conjugate of any one of paragraphs 27-33, wherein Y¹ is OR².

Paragraph 36. The conjugate of any one of paragraphs 27-33, wherein Y¹ is C(═O)R³.

Paragraph 37. The conjugate of any one of paragraphs 27-36, wherein R¹ is H.

Paragraph 38. The conjugate of any one of paragraphs 27-37, wherein n is an integer from 1 to 5, and each L¹ is selected from NH, O, C(═O), and C₁₋₆ alkylene.

Paragraph 39. The conjugate of paragraph 38, wherein m is an integer from 1 to 5, and each L² is independently selected from NH, C(═O), C₁₋₆ alkylene, —(OCH₂CH₂)ₓ—, and —(CH₂CH₂O)ₓ—.

Paragraph 40. The conjugate of paragraph 39, wherein m is 5.

Paragraph 41. The conjugate of any one of paragraphs 27-40, wherein x is an integer from 1 to 10.

Paragraph 42. The conjugate of any one of paragraphs 27-36, wherein:
R¹ is H;
n is an integer from 1 to 5, and each L¹ is selected from NH, O, C(═O), and C₁₋₆ alkylene;
m is an integer from 1 to 5, and each L² is independently selected from NH, O, C(═O), C₁₋₆ alkylene, —(OCH₂CH₂)ₓ—, and —(CH₂CH₂O)ₓ—; and
x is an integer from 2 to 10.

Paragraph 43. The conjugate of any one of paragraphs 27-42, wherein:
p is an integer from 1 to 5,
each L³ is selected from NR^{N}, O, C(═O), and C₁₋₆ alkylene,
said C₁₋₆ alkylene is optionally substituted with (L⁴)ₒ-Y³; and
R^{N} is selected from H, C₁₋₃ alkyl, and (L⁴)₀-Y³.

Paragraph 44. The conjugate of any one of paragraphs 27-43, wherein each L³ is selected from NH, O, C(═O), and C₁₋₆ alkylene, which is optionally substituted with (L⁴)ₒ-Y³.

Paragraph 45. The conjugate of paragraph 44, having formula:

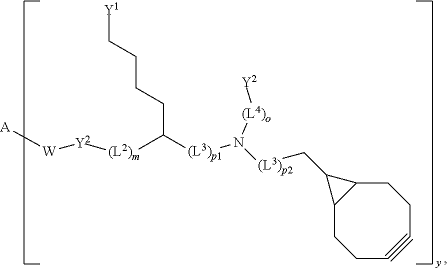

or a pharmaceutically acceptable salt thereof, wherein the sum of p1 and p2 is less than p by at least 1.

Paragraph 46. The conjugate of any one of paragraphs 27-43, wherein:
each L³ is selected from NR^{N}, O, C(═O), and C₁₋₆ alkylene, and
R^{N} is selected from H, C₁₋₃ alkyl, and (L⁴)₀-Y³.

Paragraph 47. The conjugate of paragraph 46, having formula:

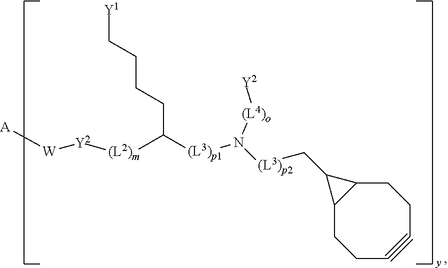

or a pharmaceutically acceptable salt thereof, wherein the sum of p1 and p2 is less than p by at least 1.

Paragraph 48. The conjugate of any one of paragraphs 27-47, wherein o is an integer from 1 to 5 and each L⁴ is independently selected from NH, O, C(═O), and C₁₋₆ alkylene.

Paragraph 49. The conjugate of any one of paragraphs 27-48, wherein Y³ is a moiety of formula (i):

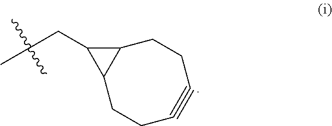 [SMILES: *CC1C2CCC#CCCC12]

Paragraph 50. The conjugate of any one of paragraphs 27-48, wherein Y³ is a moiety of formula (ii):

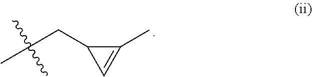 [SMILES: *CC1C=C1C]

Paragraph 51. The conjugate of any one of paragraphs 27-43, wherein each L³ is selected from NR^{N}, O, C(═O), and C₁₋₆ alkylene.

Paragraph 52. The conjugate of paragraph 51, wherein:
n is 1 and L¹ is C₁₋₆ alkylene;
m is 4, and each L² is independently selected from NH, C(═O), C₁₋₆ alkylene, —(OCH₂CH₂)ₓ—, and —(CH₂CH₂O)ₓ—; and
p is 3, and each L³ is independently selected from NH, O, and C(═O).

Paragraph 53. The conjugate of paragraph 52, wherein Formula (I) has formula:

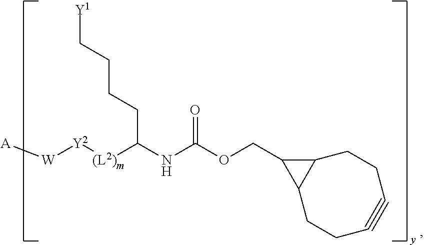

or a pharmaceutically acceptable salt thereof.

Paragraph 54. The conjugate of paragraph 53, wherein Formula (I) has formula:

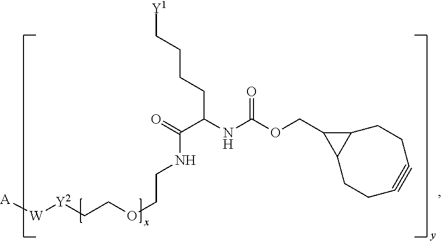

or a pharmaceutically acceptable salt thereof.

Paragraph 55. A composition comprising the conjugate of any one of paragraphs 27-54, or a pharmaceutically acceptable salt thereof, and an inert carrier.

Paragraph 56. The composition of paragraph 55, which is an aqueous solution.

Paragraph 57. A method of examining a cell or a component of a cell, the method comprising:
(i) contacting the cell with a conjugate of any one of paragraphs 27-55 comprising the fluorophore, or a pharmaceutically acceptable salt thereof, or a composition of paragraph 55 or paragraph 56;
(ii) imaging the cell with an imaging technique; and
(iii) after (ii), contacting the cell with a compound of Formula (III):

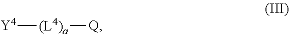

or a pharmaceutically acceptable salt thereof, wherein:
Y⁴ is selected from N₃ and a moiety of formula (iii):

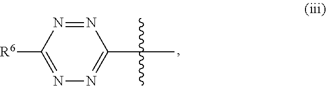

R⁶ is selected from H, C₁₋₆ alkyl, and C₁₋₆ haloalkyl, wherein said C₁₋₆ alkyl is optionally substituted with OH, NH₂, or COOH;
each L⁴ is independently selected from N(R^{N}), O, C(═O), S(═O)₂, C₁₋₆ alkylene, C₆₋₁₀ arylene, C₆₋₁₀ perfluoroarylene, —(OCH₂CH₂)ₓ—, and —(CH₂CH₂O)ₓ—;
a is an integer from 1 to 10;
each R^{N} is selected from H and C₁₋₃ alkyl;
x is an integer from 1 to 2,000; and
Q is a quencher,
wherein the contacting of step (iii) results in decrease of the fluorescence of the fluorophore in the conjugate of Formula (II), or a pharmaceutically acceptable salt thereof.

Paragraph 58. The method of paragraph 57, wherein the imaging technique is a fluorescence imaging.

Paragraph 59. The method of paragraph 57 or paragraph 58, wherein Y⁴ is N₃.

Paragraph 60. The method of paragraph 57 or paragraph 58, wherein the compound of Formula (III) has formula:

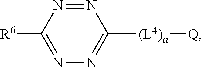

or a pharmaceutically acceptable salt thereof.

Paragraph 61. The method of any one of paragraphs 57-60, wherein R⁶ is H.

Paragraph 62. The method of any one of paragraphs 57-60, wherein R⁶ is C₁₋₆ alkyl, optionally substituted with OH, NH₂, or COOH.

Paragraph 63. The method of any one of paragraphs 57-62, a is an integer from 1 to 7, and each L⁴ is independently selected from NH, C(═O), C₁₋₆ alkylene, C₆₋₁₀ arylene, C₆₋₁₀ perfluoroarylene, and —(CH₂CH₂O)ₓ—.

Paragraph 64. The method of paragraph 57, wherein the compound of Formula (III) is selected from any one of the following compounds:

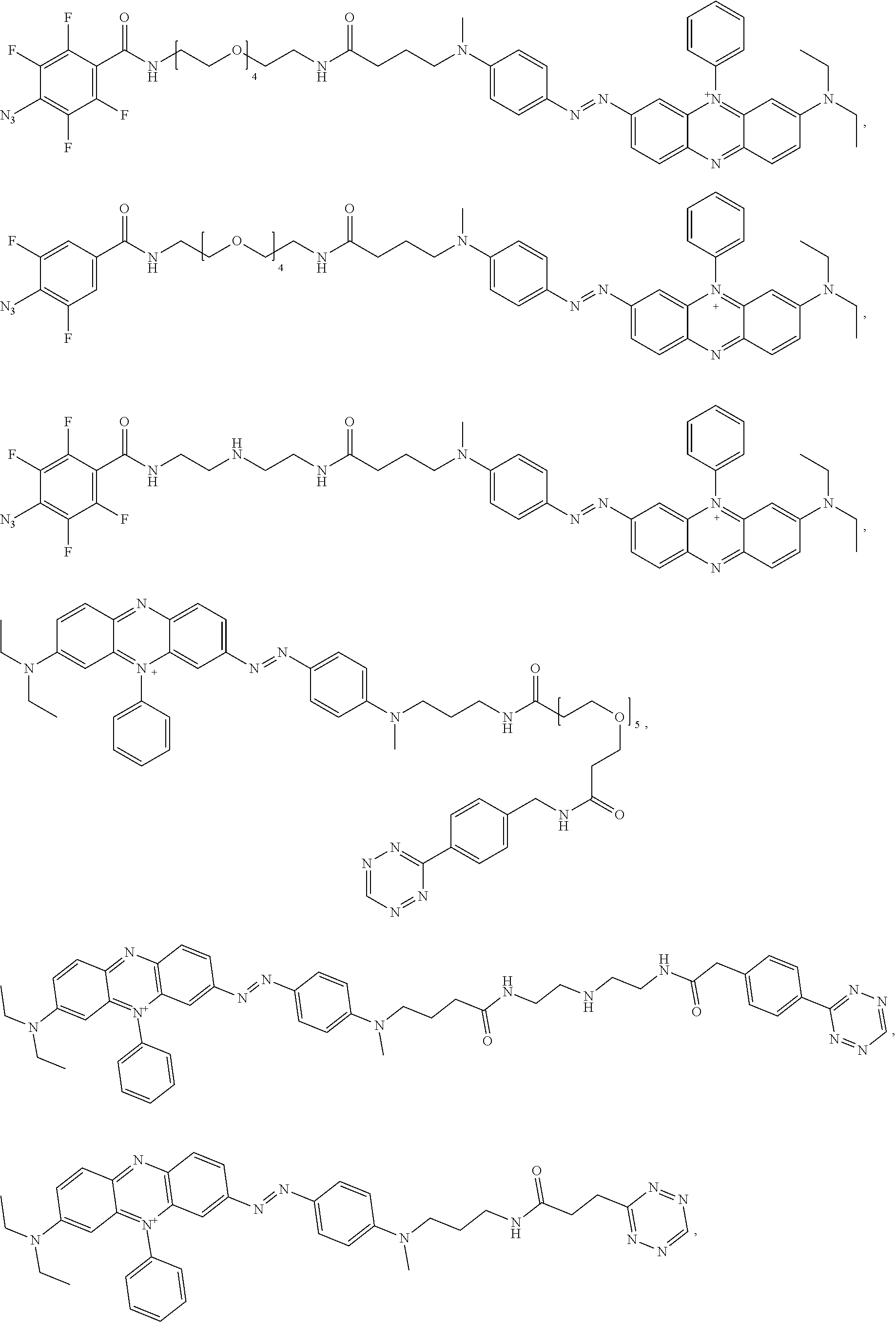

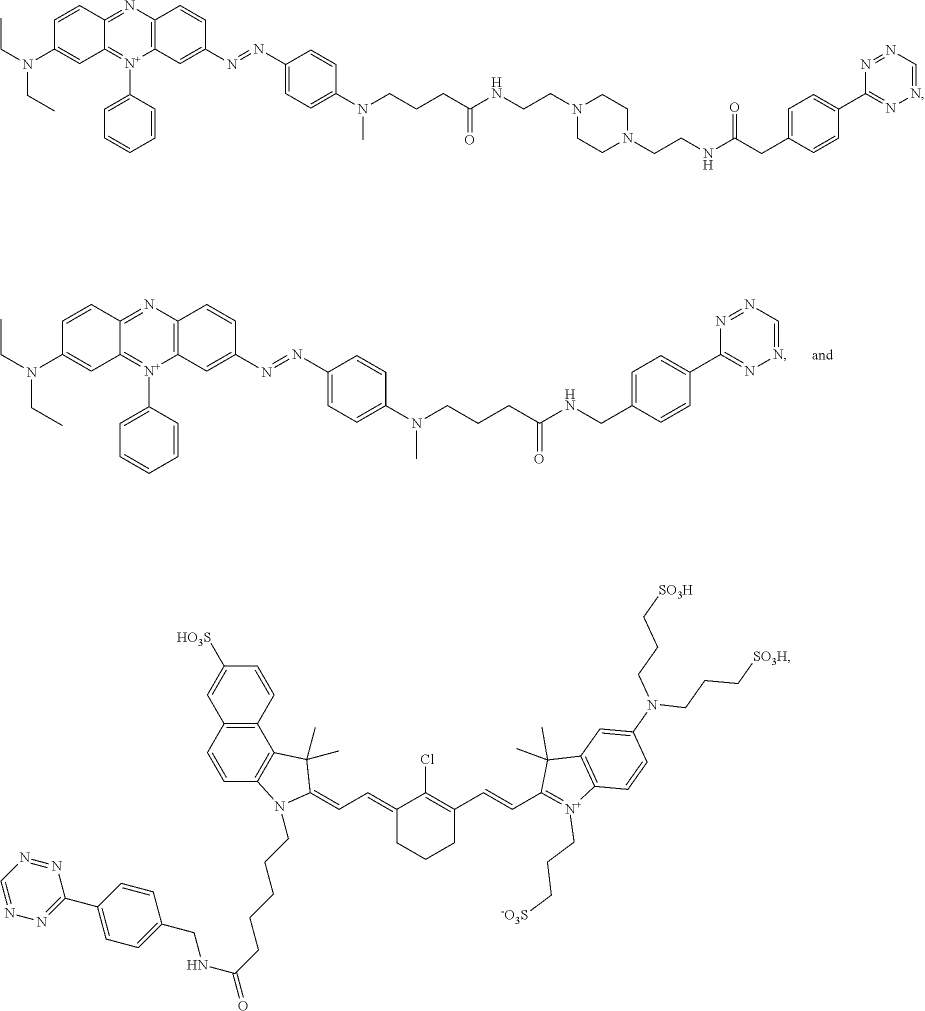

or a pharmaceutically acceptable salt thereof.

Paragraph 65. A method selected from:
profiling a cell;
examining a cell using a cytometry technique;
diagnosing a disease or condition of a subject by examining pathology of a cell obtained from the subject;
monitoring progression of disease or condition of a subject by examining pathology of a cell obtained from the subject; and
detecting a disease biomarker in a cell;
the method comprising:
(i) obtaining a cell from the subject; and
(ii) examining the cell according to the method of any one of paragraphs 57-64.

Paragraph 66. The method of paragraph 65, wherein the cell is obtained from the subject using image-guided biopsy, fine needle aspiration (FNA), surgical tissue harvesting, punch biopsy, liquid biopsy, brushing, swab, touch-prep, fluid aspiration or blood analysis.

Paragraph 67. The method of paragraph 65 or paragraph 66, wherein the cytometry technique is selected from image cytometry, holographic cytometry, Fourier ptychography cytometry, and fluorescence cytometry.

Paragraph 68. The method of any one of paragraphs 65-67, wherein the cell is selected from a cancer cell, an immune system cell, and a host cell.

Paragraph 69. The method of any one of paragraphs 65-68, wherein the disease or condition is cancer.

Paragraph 70. The method of paragraph 69, wherein the cancer is selected from lymphoma, breast cancer, skin cancer, lymphoma nodes, head and neck cancer, and oral cancer.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

What is claimed is:

1. A compound of Formula (I): 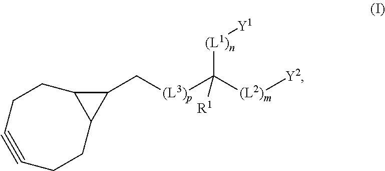
or a pharmaceutically acceptable salt thereof, wherein:
R¹ is selected from H, C₁₋₃ alkyl, and C₁₋₃ haloalkyl;
each L¹ is independently selected from N(R^{N}), O, C(═O), S(═O)₂, C₁₋₆ alkylene, —(OCH₂CH₂)ₓ—, and —(CH₂CH₂O)ₓ—;
n is an integer from 1 to 10;
each L² is independently selected from N(R^{N}), O, C(═O), S(═O)₂, C₁₋₆ alkylene, —(OCH₂CH₂)ₓ—, and —(CH₂CH₂O)ₓ—;
m is an integer from 1 to 10;
each L³ is independently selected from N(R^{N}), O, C(═O), S(═O)₂, C₁₋₆ alkylene, —(OCH₂CH₂)ₓ—, and —(CH₂CH₂O)ₓ—;
wherein each of said C₁₋₆ alkylene groups within L¹, L², or L³ is optionally substituted with (L⁴)ₒ-Y³;
each R^{N} is independently selected from H, C₁₋₃ alkyl, C₁₋₃ haloalkyl, and (L⁴)ₒ-Y³;
each L⁴ is independently selected from N(R^{N}), O, C(═O), S(═O)₂, C₁₋₆ alkylene, —(OCH₂CH₂)ₓ—, and —(CH₂CH₂O)ₓ—;
each R^{N1} is independently selected from H, C₁₋₃ alkyl, and C₁₋₃ haloalkyl;
p is an integer from 1 to 20;
o is an integer from 1 to 10;
each x is independently an integer from 1 to 2,000;
each Y³ is independently selected from a moiety of formula (i) and a moiety of formula (ii): 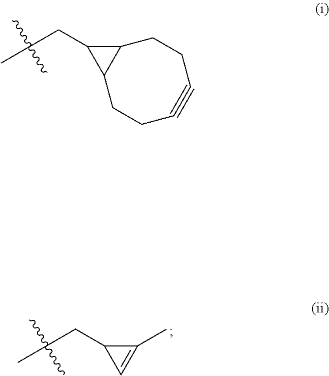
Y¹ is selected from NR^{c1}R^{1A}, OR², and C(═O)R³;
R^{1A} selected from H, an amine protecting group, and a fluorophore;
R² is selected from H, an alcohol protecting group, and a fluorophore;
R³ is selected from OR^{a1} and a fluorophore;
Y² is selected from C(═O)OR^{a1}, NR^{c1}R^{1A}, OR⁵; and a group reactive with a side chain of an amino acid of a protein;
R^{a1} is selected from H and a carboxylic acid protecting group;
R^{c1} is selected from H, C₁₋₃ alkyl, and C₁₋₃ haloalkyl;
R⁴ is selected from H and an amine protecting group; and
R⁵ is selected from H and an alcohol protecting group.

2. The compound of claim 1, R¹ is H.

3. The compound of claim 1, wherein n is an integer from 1 to 5, and each L¹ is selected from NH, O, C(═O), and C₁₋₆ alkylene.

4. The compound of claim 1, wherein m is an integer from 1 to 5, and each L² is independently selected from NH, C(═O), C₁₋₆ alkylene, —(OCH₂CH₂)ₓ—, and —(CH₂CH₂O)ₓ—.

5. The compound of claim 1, wherein:
R¹ is H;
n is an integer from 1 to 5, and each L¹ is selected from NH, O, C(═O), and C₁₋₆ alkylene;
m is an integer from 1 to 5, and each L² is independently selected from NH, O, C(═O), C₁₋₆ alkylene, —(OCH₂CH₂)ₓ—, and —(CH₂CH₂O)ₓ—; and
x is an integer from 2 to 10.

6. The compound of claim 1, wherein:
p is an integer from 1 to 5,
each L³ is selected from NR^{N}, O, C(═O), and C₁₋₆ alkylene,
said C₁₋₆ alkylene is optionally substituted with (L⁴)ₒ-Y³; and
R^{N} is selected from H, C₁₋₃ alkyl, and (L⁴)₀-Y³.

7. The compound of claim 1, wherein each L³ is selected from NH, O, C(═O), and C₁₋₆ alkylene, which is optionally substituted with (L⁴)₀-Y³.

8. The compound of claim 7, having formula: 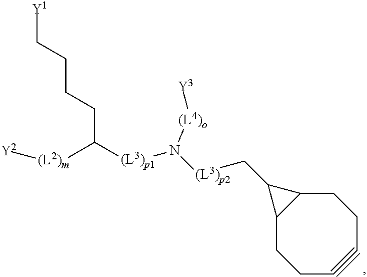
or a pharmaceutically acceptable salt thereof, wherein the sum of p1 and p2 is less than p by at least 1.

9. The compound of claim 1, wherein:
each L³ is selected from NR^{N}, O, C(═O), and C₁₋₆ alkylene, and
R^{N} is selected from H, C₁₋₃ alkyl, and (L⁴)₀-Y³.

10. The compound of claim 9, having formula: 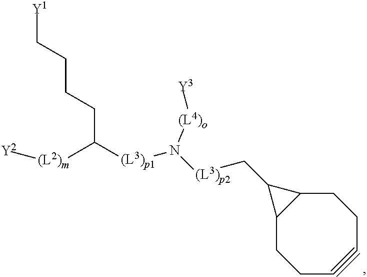
or a pharmaceutically acceptable salt thereof, wherein the sum of p1 and p2 is less than p by at least 1.

11. The compound of claim 1, wherein o is an integer from 1 to 5 and each L⁴ is independently selected from NH, O, C(═O), and C₁₋₆ alkylene.

12. The compound of claim 11, wherein Y³ is a moiety of formula (i):

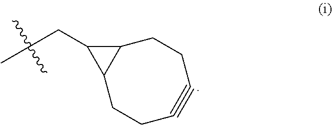 [SMILES: *CC1C2CCC#CCCC12]

13. The compound of claim 11, wherein Y³ is a moiety of formula (ii):

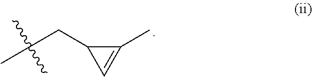 [SMILES: *CC1C=C1C]

14. The compound of claim 1, wherein each L³ is selected from NR^{N}, O, C(═O), and C₁₋₆ alkylene.

15. The compound of claim 14, wherein:
n is 1 and L¹ is C₁₋₆ alkylene;
m is 4, and each L² is independently selected from NH, C(═O), C₁₋₆ alkylene, —(OCH₂CH₂)ₓ—, and —(CH₂CH₂O)ₓ—; and
p is 3, and each L³ is independently selected from NH, O, and C(═O).

16. The compound of claim 15, wherein Formula (I) has formula: 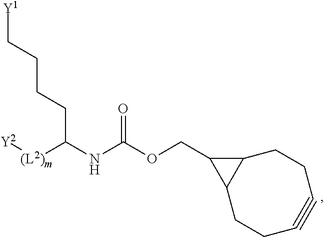
or a pharmaceutically acceptable salt thereof.

17. The compound of claim 15, wherein Formula (I) has formula: 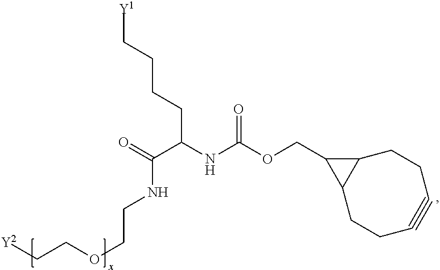
or a pharmaceutically acceptable salt thereof.

18. The compound of claim 1, wherein:
Y¹ is NHR^{1A}; and
Y² is selected from C(═O)OR^{a1} and a group reactive with a side chain of an amino acid of a protein.

19. The compound of claim 1, wherein:
Y¹ is NH₂; and
Y² is C(═O)OH.

20. The compound of claim 1, wherein:
Y¹ is NHR^{1A};
R¹ is an amine protecting group; and
Y² is C(═O)OH.

21. The compound of claim 1, wherein:
Y¹ is NHR^{1A};
R¹ is a fluorophore; and
Y² is C(═O)OH.

22. The compound of claim 1, wherein:
Y¹ is NHR^{1A};
R¹ is a fluorophore;
Y² is C(═O)OR^{a1}; and
R^{a1} is a carboxylic acid protecting group.

23. The compound of claim 1, wherein:
Y¹ is NHR^{1A};
R¹ is a fluorophore; and
Y² is a group reactive with a side chain of an amino acid of a protein.

24. The compound of claim 1, wherein the compound of Formula (I) is selected from any one of the following compounds: 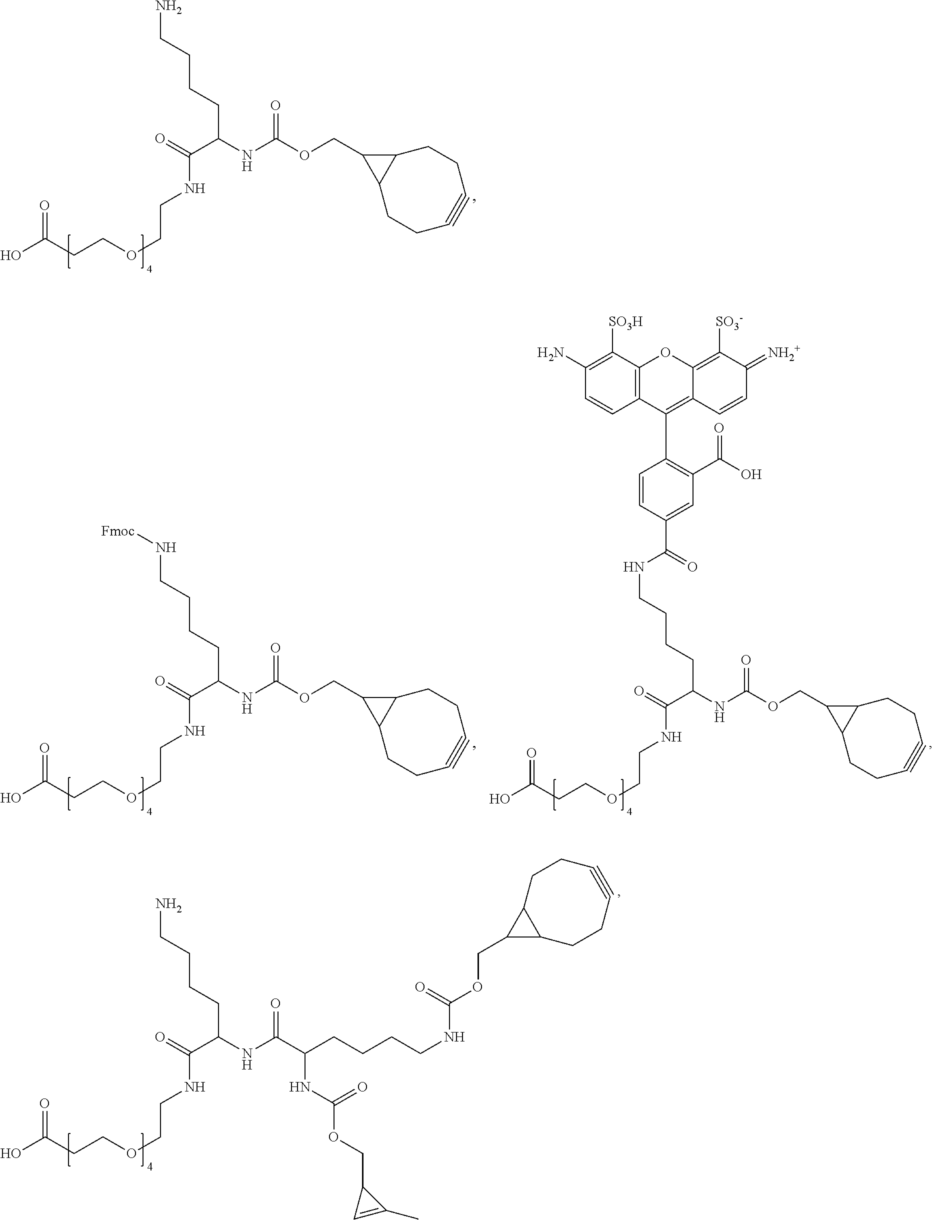 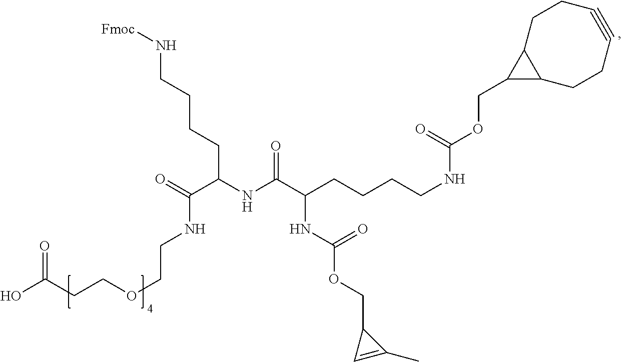 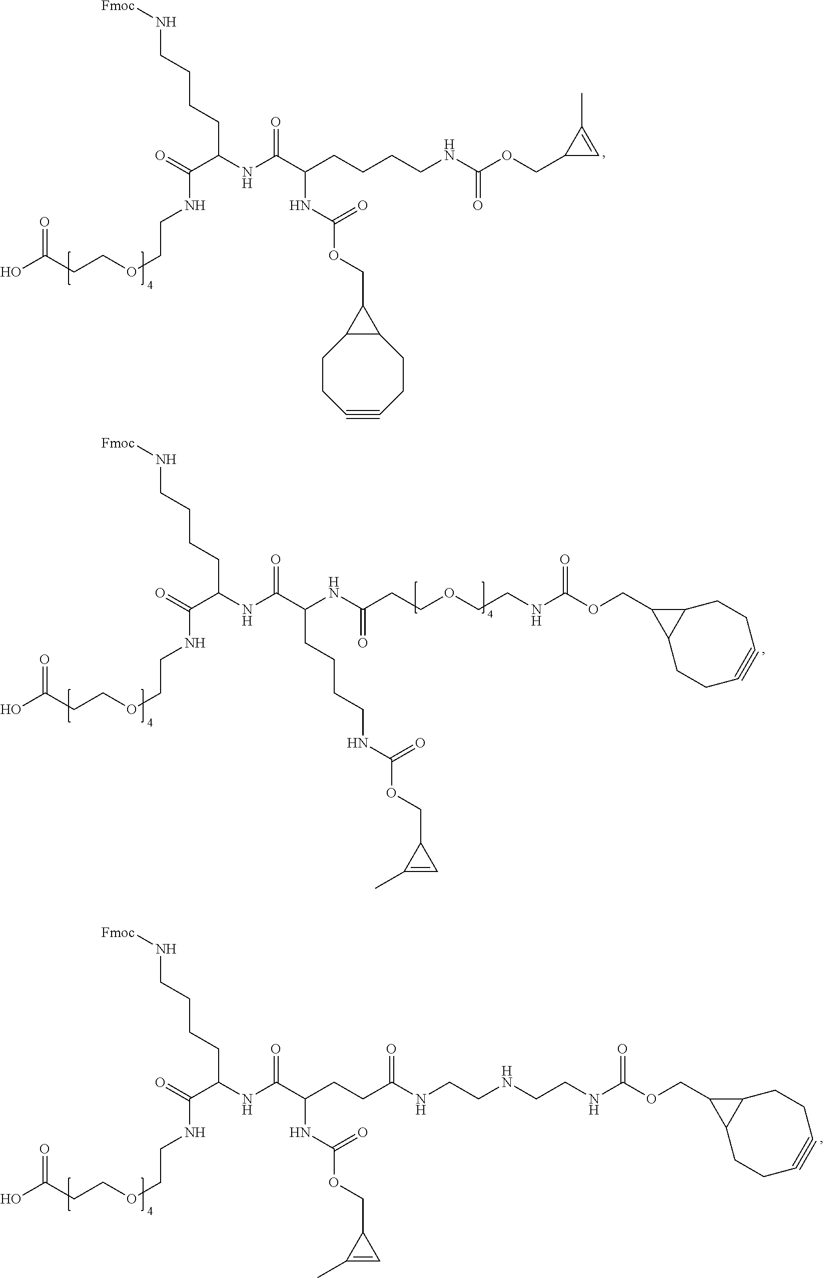 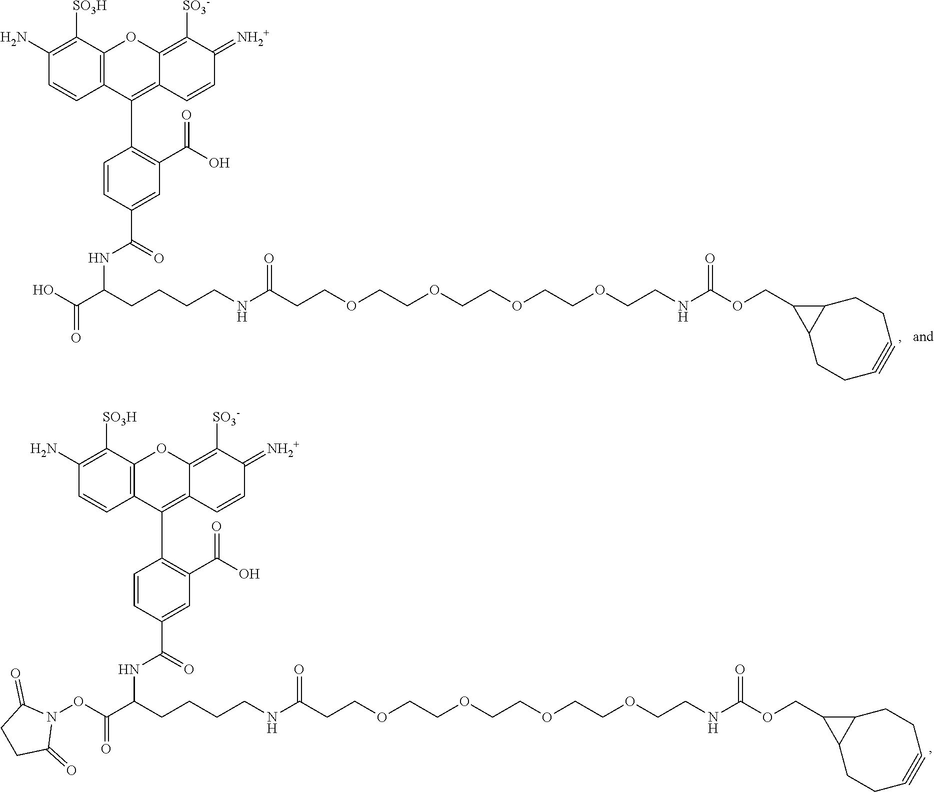
or a pharmaceutically acceptable salt thereof.

25. A protein conjugate of Formula (II): 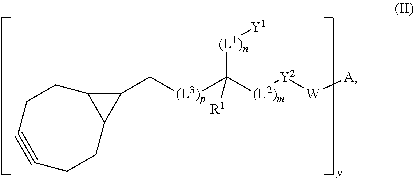
or a pharmaceutically acceptable salt thereof, wherein:
A is a protein;
y is an integer from 1 to 10;
R¹ is selected from H, C₁₋₃ alkyl, and C₁₋₃ haloalkyl;
each L¹ is independently selected from N(R^{N}), O, C(═O), S(═O)₂, C₁₋₆ alkylene, —(OCH₂CH₂)ₓ—, and —(CH₂CH₂O)ₓ—;
n is an integer from 1 to 10;
each L² is independently selected from N(R^{N}), O, C(═O), S(═O)₂, C₁₋₆ alkylene, —(OCH₂CH₂)ₓ—, and —(CH₂CH₂O)ₓ—;
m is an integer from 1 to 10;
each L³ is independently selected from N(R^{N}), O, C(═O), S(═O)₂, C₁₋₆ alkylene, —(OCH₂CH₂)ₓ—, and —(CH₂CH₂O)ₓ—;
wherein each of said C₁₋₆ alkylene groups within L¹, L², or L³ is optionally substituted with (L⁴)ₒ-Y³;
each R^{N} is independently selected from H, C₁₋₃ alkyl, C₁₋₃ haloalkyl, and (L⁴)ₒ-Y³;
each L⁴ is independently selected from N(R^{N1}), O, C(═O), S(═O)₂, C₁₋₆ alkylene, —(OCH₂CH₂)ₓ—, and —(CH₂CH₂O)ₓ—;
each R^{N1} is independently selected from H, C₁₋₃ alkyl, and C₁₋₃ haloalkyl;
p is an integer from 1 to 20;
o is an integer from 1 to 10;
each x is independently an integer from 1 to 2,000;
each Y³ is independently selected from a moiety of formula (i) and a moiety of formula (ii): 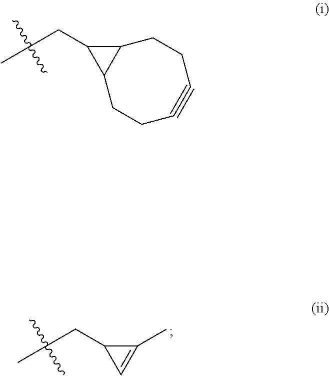
Y¹ is selected from NR^{c1}R^{1A}, OR², and C(═O)R³;
R^{1A}, R², and R³ are each independently selected from a fluorophore;
R^{c1} is selected from H, C₁₋₃ alkyl, and C₁₋₃ haloalkyl;
each W is selected from:
(i) O of a side chain of serine, threonine, or tyrosine of the protein A;
(ii) S of a side chain of cysteine of the protein A;
(iii) NH of a side chain of lysine of the protein A; and
(iv) C(═O) of a side chain of aspartic acid or glutamic acid of the protein A; and
Y² is a residue of a group which, prior to conjugation with the protein A, was a group reactive with a side chain of an amino acid of the protein A.

26. A composition comprising the conjugate of claim 25, or a pharmaceutically acceptable salt thereof, and an inert carrier.

27. A method of examining a cell or a component of a cell, the method comprising:
(i) contacting the cell with a conjugate of claim 25 comprising the fluorophore, or a pharmaceutically acceptable salt thereof, or a composition of claim 26;
(ii) imaging the cell with an imaging technique; and
(iii) after (ii), contacting the cell with a compound of Formula (III): 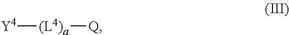
or a pharmaceutically acceptable salt thereof, wherein:
Y⁴ is selected from N₃ and a moiety of formula (iii): 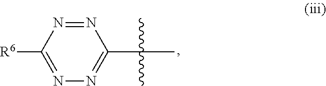
R⁶ is selected from H, C₁₋₆ alkyl, and C₁₋₆ haloalkyl, wherein said C₁₋₆ alkyl is optionally substituted with OH, NH₂, or COOH;
each L⁴ is independently selected from N(R^{N}), O, C(═O), S(═O)₂, C₁₋₆ alkylene, C₆₋₁₀ arylene, C₆₋₁₀ perfluoroarylene, —(OCH₂CH₂)ₓ—, and —(CH₂CH₂O)ₓ—;
a is an integer from 1 to 10;
each R^{N} is selected from H and C₁₋₃ alkyl;
x is an integer from 1 to 2,000; and
Q is a quencher,
wherein the contacting of step (iii) results in decrease of the fluorescence of the fluorophore in the conjugate of Formula (II), or a pharmaceutically acceptable salt thereof.

28. The method of claim 27, wherein the compound of Formula (III) has formula: 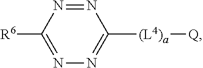
or a pharmaceutically acceptable salt thereof.

29. The method of claim 27, wherein the compound of Formula (III) is selected from any one of the following compounds: 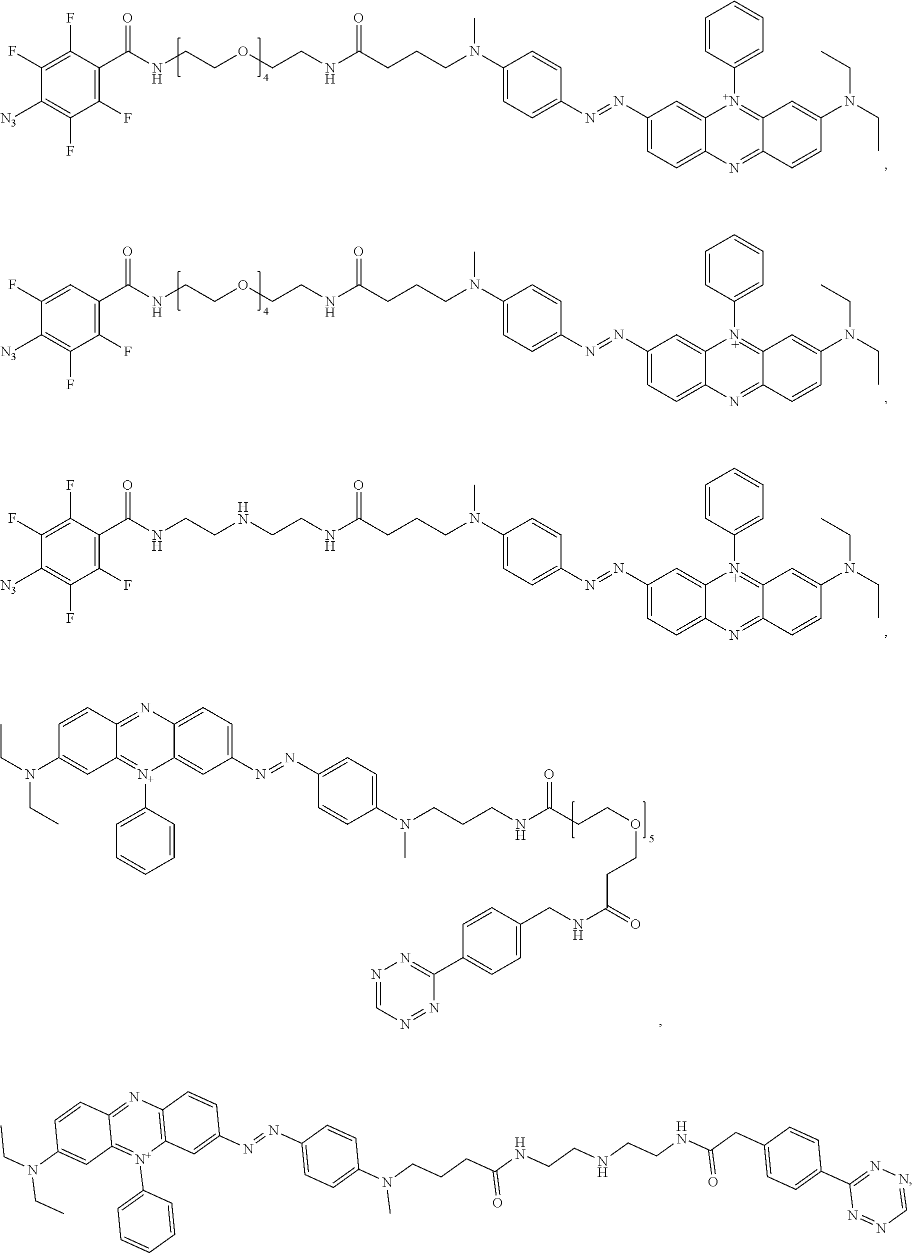 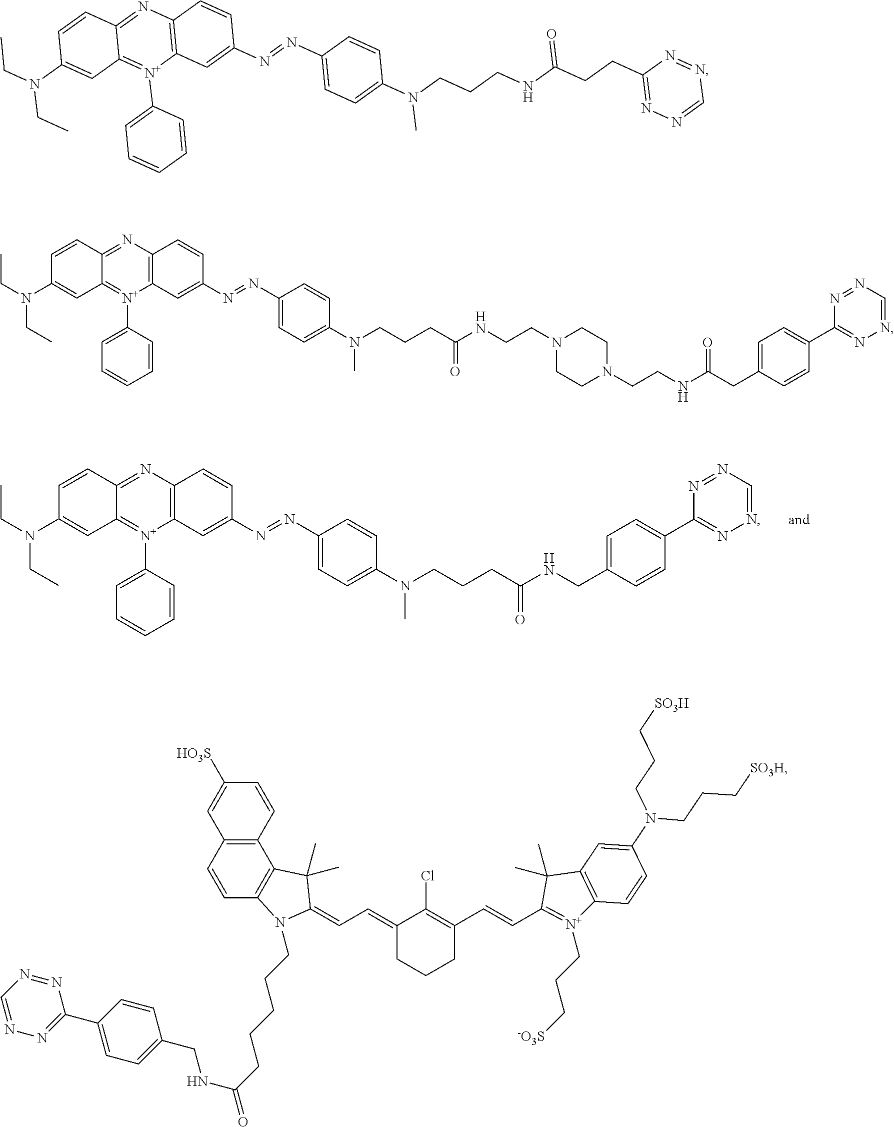
or a pharmaceutically acceptable salt thereof.

30. A method selected from:
profiling a cell;
examining a cell using a cytometry technique;
diagnosing a disease or condition of a subject by examining pathology of a cell obtained from the subject;
monitoring progression of disease or condition of a subject by examining pathology of a cell obtained from the subject; and
detecting a disease biomarker in a cell;
the method comprising:
(i) obtaining a cell from the subject; and
(ii) examining the cell according to the method of claim 27.
